(19) United States
(12) Patent Application Publication
(10) Pub. No.: US 2024/0207914 A1
(43) Pub. Date: Jun. 27, 2024

(54) POLYMER DEGRADING ENZYMES

(71) Applicants: Alliance for Sustainable Energy, LLC, Golden, CO (US); UNIVERSITY OF PORTSMOUTH, Portsmouth (GB)

(72) Inventors: Erika Marie ERICKSON, Golden, CO (US); Gregg Tyler BECKHAM, Golden, CO (US); Japheth Emi GADO, Golden, CO (US); John E. MCGEEHAN, Portsmouth (GB); Christina Marie PAYNE, Arlington, VA (US)

(21) Appl. No.: 18/556,064

(22) PCT Filed: Apr. 20, 2022
(86) PCT No.: PCT/US22/25624

§ 371 Date: Oct. 18, 2023

Related U.S. Application Data
(60) Provisional application No. 63/177,334, filed on Apr. 20, 2021.
(60) Provisional application No. 63/297,529, filed on Jan. 7, 2022.

(51) Int. Cl. B09B 3/60; C12N 1/20; C12N 9/18; C12N 15/70; B09B 101/75

(52) U.S. Cl. CPC B09B 3/60; C12N 1/20; C12N 9/18; C12N 15/70; C12Y 301/01; B09B 2101/75

(57) ABSTRACT

Disclosed herein are PET hydrolase enzymes, and their nucleic acid and amino acid sequences. A number of candidates have been identified with detectable, quantifiable activity on PET and these enzymes possess desirable traits that are leveraged in the design and engineering of enzyme formulations targeted to degrade specific polymers. These enzymes have measurable PET degrading activity and, in an embodiment, may be active polyester polyurethanes.

### CROSS-REFERENCE TO RELATED APPLICATIONS

This application is a national phase entry under 35 U.S.C. § 371 and claims priority to PCT application number PCT/US2022/025624 filed 20 Apr. 2022 which claims priority under 35 U.S.C. § 119 to U.S. provisional patent application No. 63/177,334 filed on 20 Apr. 2021 and 63/297,529 filed on 7 Jan. 2022, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Plastics accumulation in nature represents a global environmental crisis. In response, microbes are evolving the capacity to utilize synthetic polymers as carbon and energy sources. Synthetic polymers pervade all aspects of modern life, due to their low cost, high durability, and impressive extents of tunability. Originally developed to avoid the use of animal-based products, plastics have now become so widespread that their leakage into the biosphere and accumulation in landfills is creating a global-scale environmental crisis. Indeed, plastics have been found widespread in the world's oceans, in the soil, and more recently, microplastics have been reported even entrained in the air.

The accumulation of plastics waste in landfills and throughout the natural environment represents a global pollution crisis. Concurrently, petrochemical-derived plastics manufacturing and consumption are also major contributors to global greenhouse gas (GHG) emissions. These dual challenges in end-of-life management and the manufacturing of plastics have prompted a surge of activity in the development of chemical recycling technologies, wherein synthetic polymers are deconstructed to intermediates that can be recycled into the same material in a closed-loop process or converted into alternative products in an open-loop process. One of the most commonly used and discarded plastics is poly(ethylene terephthalate) (PET), which is a polyester employed in single-use beverage bottles, textiles, and packaging, among other applications. Given its ubiquity in consumer plastics and the relative ease of ester bond cleavage, PET is among the most well-studied polymers for chemical recycling, and thermal, catalytic, and biocatalytic approaches for PET recycling are currently being pursued. For biocatalytic conversion of PET, the use of hydrolase enzymes has witnessed major advances especially in the last decade, both in terms of advancing the industrial relevance of this approach, as well as the discovery of natural microbial systems that respond to the presence of PET in the biosphere.

Thirty-six serine hydrolase family enzymes have been experimentally confirmed to deconstruct PET to its constituent monomers, terephthalic acid (TPA) and ethylene glycol (EG). Most known PET hydrolases are cutinases, lipases, and carboxylesterases (Enzyme Commission 3.1.1.-). Based upon pioneering enzyme discoveries, multiple structural biology, protein engineering, and enzyme screening efforts have aimed to identify the necessary features for an enzyme to hydrolyze PET and to improve these enzymes for industrial application. Notably, the most efficient PET-degrading biocatalysts are thermostable enzymes that exhibit optimal PET hydrolysis activity near the PET glass transition temperature (PET Tg values can range from) ˜65-80° C. For example, others have engineered thermotolerant leaf-branch compost cutinase (LCC) variants that displayed substantial performance improvements for amorphous PET hydrolysis, and similar protein engineering efforts have achieved improved thermotolerance in Thermobifida cutinases, among others recently reported a new thermotolerant cutinase with high structural similarity to LCC that also exhibits excellent PET hydrolysis performance on amorphous substrates. Given the need for activity under thermophilic conditions for effective PET hydrolysis, multiple protein engineering efforts have also been conducted to improve the thermal stability of the mesophilic Ideonella sakaiensis PETase. These studies have made considerable advances, but progress could be potentially accelerated further via discovery of a broader diversity of enzyme scaffolds with PET hydrolytic activity.

To date, the sequence and structural features that confer PET hydrolysis activity are not yet fully understood, both within and beyond the sequence space explored to date. Similarly, the diversity of enzymes naturally able to hydrolyze PET remains unclear. To address these questions, others have applied a Hidden Markov Model (HMM) in 2018 to search metagenomic databases for potential PET hydrolases. They identified 504 putative PET hydrolases, based on known sequences at the time, and further confirmed PET hydrolysis in four new enzymes. They noted that PET hydrolysis activity, based on the enzymes reported then, is likely quite rare in nature. As the authors discussed, there remains an urgent need to further develop the suite of known PET-active enzymes from natural diversity.

### SUMMARY

In an aspect, disclosed herein are PET hydrolase enzymes, nucleic acid and amino acid sequences for PET hydrolase enzymes and methods for using algorithms to predict tertiary and quaternary structures of the expressed PET hydrolase enzymes useful for generating non-naturally occurring PET hydrolase enzymes with improved activity and stability. In an embodiment the PET hydrolase enzymes disclosed herein are useful for degrading PET. In an embodiment, the enzymes disclosed herein are useful for degrading polyester polyurethanes.

Other objects, advantages, and novel features of the present invention will become apparent from the following detailed description of the invention when considered in conjunction with the accompanying drawings.

### BRIEF DESCRIPTION OF THE DRAWINGS

FIGS. 1A, 1B depict bioinformatics and machine learning to derive PET hydrolase sequences from natural diversity. FIG. 1A depicts minimum-evolution phylogenetic tree of 74 PET hydrolase candidates selected by HMM and ML. Sequences retrieved from environmental (meta)genomes in JGI IMG with lower HMM scores (groups 1 to 3) are notably diverse compared to the sequences that comprise the rest of the tree (groups 4-7). The symbols around the tree show expression, activity, and previously reported PET activity. FIG. 1B depicts a Sequence Similarity Network (SSN) of enzymes with experimentally confirmed PET hydrolase activity. Edges represent pairwise BLAST similarity with E-value <1e⁻¹⁰. The SSN clusters are consistent with the associated families in the ESTHER database and with the phylogenetic groups in FIG. 1A, and show that most reported PET hydrolases fall in the polyester-lipase-cutinase family.

FIGS. 2A, 2B depict enzyme activities. FIG. 2A depicts heat map profiles of pH and temperature screening on amorphous PET film for a diverse selection of enzymes and two control enzymes. The heat map gradient indicates the extent of measured product release up to 500 mg/L of total aromatic products after 96 h reaction time. FIG. 2B depicts a log-plot of the sum of aromatic products measured after 168 h reaction time as measured from time-course experiments using crystalline PET powder (open squares) and amorphous PET film (black squares) as substrates. Reaction conditions used for time-course experiments correspond to the pH and temperature resulting in the highest product release observed in screening reactions, and are listed in Table 5. For all enzymatic reactions shown in panels A-B, the enzyme loading was 0.7 mg enzyme/g PET and the solids loading was 2.9% (29 g/L). The reaction products were quantified with HPLC, and the results show the sum of aromatic products, including BHET, MHET, and TPA.

FIGS. 3A, 3B, and 3C depict the structural diversity of PET-active enzymes from phylogenetic groups. All structural models are shown to scale, rendered as cartoons with transparent accessible surface areas and putative active sites highlighted with the Ser-His-Asp catalytic triad in red sticks. FIG. 3A depicts PET hydrolase scaffolds identified from mesophilic (top, I. sakaisiensis PETase, PDB ID 6EQE (32)) and thermophilic (middle, LCC, PDB ID 4EB0 (29), and bottom, T. fusca cutinase 1 DSM44342 (703)) sources occupy a narrow structural space with highly conserved α/β hydrolase folds. FIG. 3B depicts a selection of representatives from more distant phylogenetic groups reveals multiple additional and alternative structural features with substantial increases (102) and reductions (307) in the core fold. FIG. 3C depicts several additional distinct domains were revealed, including a Peripheral Subunit-Binding Domain (PSBD) and a Family 35 carbohydrate binding module (CBM).

FIGS. 4A, 4B, 4C, and 4D depict increasing degrees of structural diversity across phylogenic groups. FIG. 4A depicts conserved canonical folds with surface residue changes in groups 5 and 6. Electrostatic surface representations are colored with a gradient from red (acidic) at −7 kT/e to blue (basic) at 7 kT/e (where k is Boltzmann's constant, T is temperature, and e is the charge on an electron). The general location of active sites is indicated with a star, and known (LCC) and predicted catalytic triad residues are shown as stick representations in the corresponding images below. FIG. 4B depicts accessory lid domains in group 2 enzymes. The peptidase-like core is generally conserved across this group, with the exception of a few helical deletions distal from the predicted active sites. Examples of alternative lid domains are highlighted in green. FIG. 4C depicts mini-PETases are created from large core deletions to the canonical fold. LCC is shown in the middle column (yellow) as a cartoon with the catalytic triad highlighted in red, and a surface representation below with a PET trimer (blue) docked in the active site cleft. A comparison with 307 on the left (cartoon shown without the lid domain for clarity) reveals the extent of the core deletion, removing four of the eight β-strands and corresponding helices. A comparison with 305 on the right reveals an almost complementary set of deletions. Enzyme 307 approximates the left half of the LCC core domain while 305 approximates the right half. These major rearrangements generate alternative binding clefts and docking studies predict vastly different binding modes (PET trimers in blue). FIG. 4D depicts an alternative enzyme family for PET hydrolysis. The enzymes 101 (left) and 102 (right) are colored according to the 3-domain arrangement in the Geobacillus stearothermophilus carboxylesterase EST55 (PDB ID 20GT). Both enzymes display a truncated version of the catalytic domain (pink) compared to EST55 and have modified versions of the α/β domain (blue). Only enzyme 101 has a version of the regulatory domain, the absence of which in 102 disrupts the formation of the canonical active site (locations highlighted with red dashes). While the catalytic Ser and Glu residues are conserved between EST55 and 101 (pink and yellow sticks), there is no direct substitute for the His residue. In enzyme 102, only the catalytic Ser is position is conserved, although there are other candidate residues that could potentially form a productive triad.

FIGS. 5A, 5B, 5C, 5D, and 5E depict a time-course plots comparing product release from amorphous PET film and crystalline PET powder over 168 h reaction time. Error bars represent the standard deviation of reactions measured in triplicate. FIG. 5A depicts a comparison of control enzymes using peak activity reaction conditions from screening on amorphous PET film. FIG. 5B depicts a comparison of selected candidate enzymes using peak activity conditions from screening on amorphous PET film. FIG. 5C depicts a comparison of two reaction conditions for enzyme 606 showing that 606 has higher activity in more alkaline reaction conditions. FIG. 5D depicts a comparison of two reaction conditions for enzyme 611. Enzyme 611 is more selective for crystalline PET powder compared to amorphous PET in both conditions tested. FIG. 5E depicts a comparison of two reaction conditions for enzyme 704, showing that while 704 prefers a more alkaline reaction environment (pH 9), comparable activity is achieved even at pH 7.

### DETAILED DESCRIPTION

Industrial adoption of new plastics recycling and upcycling technologies could incentivize the reclamation of waste plastics and reduce greenhouse gas emissions from virgin plastics manufacturing. To this end, the use of hydrolase enzymes for polyester recycling has witnessed a surge of interest from the biotechnology community. Process analysis has predicted that enzymatic PET recycling could have both substantial economic and sustainability benefits if deployed at scale. Thus far, approximately 36 related enzymes have been demonstrated to breakdown PET to its monomers, prompting the search for more distant and diverse functional biocatalysts for PET hydrolysis. Disclosed herein are methods and to identify distantly related enzymes with high-temperature PET activity, thus providing a rich biochemical and structural resource for further engineering of enzymatic PET hydrolysis.

The leakage of plastics into the environment on a planetary scale has led to the subsequent discovery of multiple biological systems able to convert man-made polymers for use as a carbon and energy source. On the basis of these natural systems able to degrade synthetic plastics, the environmental microbiology community is interested to understand how natural enzymes evolve to convert non-natural substrates, which in turn will enable these systems to be used for biotechnology applications towards a circular materials economy.

New recycling solutions are critically needed to mitigate waste plastics pollution. To that end, the enzymatic deconstruction of a ubiquitous polyester, poly(ethylene terephthalate) (PET), is under intense investigation, particularly given the promise of a biological recycling approach that can depolymerize PET to its constituent monomers near the polymer glass transition temperature)(˜70° C. To date, reported PET hydrolases have been sourced from a relatively narrow sequence space. To enable such an enzymatic recycling approach, we sought to identify additional biocatalysts for PET deconstruction from natural diversity. In this work, we used bioinformatics and machine learning to identify 74 putative thermotolerant PET hydrolases, based on a set of known PET hydrolyzing enzymes. We successfully expressed, purified, and assayed 52 enzymes from seven distinct phylogenetic groups, and within this set, we observed PET hydrolysis activity in 37 enzymes in reactions spanning a range of pH from 4.5-9.0 and temperatures from 30-70° C. We conducted biophysical characterization and PET hydrolysis time-course reactions with the best-performing enzymes, which demonstrated that some enzymes exhibit higher specificity towards crystalline PET rather than the commonly observed preference for amorphous PET. We employed X-ray crystallography and the AlphaFold artificial intelligence-based protein structure prediction algorithm to interrogate the enzyme architectures, which revealed both protein folds and accessory domains not previously associated with PET deconstruction. Taken together, this study expands the number and structural diversity of thermotolerant protein scaffolds for PET hydrolysis, which can enable further engineering for enzymatic PET recycling and upcycling.

In an embodiment, an objective of the current disclosure is to expand the catalog of thermotolerant PET hydrolase scaffolds. To this end, we combined an HMM approach with machine learning (ML) to predict the temperature where the enzyme would be optimally active based on its sequence. In doing so, we selected 74 putative thermotolerant PET hydrolases for experimental screening, sourced from seven distinct phylogenetic groups, including several from which no PET hydrolysis activity has been previously reported to our knowledge. Expression and purification trials for each enzyme were conducted, and the proteins successfully expressed were screened for amorphous PET hydrolysis as a function of pH and temperature. For the best-performing enzymes from each group, we conducted both thermal characterization to measure the melting temperature (Tm), and time-course reactions using crystalline PET powder and amorphous PET films as substrate to ascertain differences in reactivity as a function of substrate properties. Lastly, we combined X-ray crystallography and AlphaFold for structural characterization of all 74 enzymes to gain insights into the structure-activity relationships that confer PET hydrolytic activity. Taken together, this work suggests that PET hydrolytic activity can be sourced from a wider range of natural diversity than previously reported and expands the number of enzyme structural scaffolds for thermotolerant PET hydrolase engineering.

Bioinformatics and ML enables identification of 74 putative thermotolerant PET hydrolases from seven distinct phylogenetic groups. Similar to other successes in identifying PET hydrolases with HMM, we constructed an HMM from 17 characterized enzymes that were confirmed to exhibit PET hydrolysis activity as of December 2018, and applied the HMM to search sequences in the National Center for Biotechnology Information (NCBI) non-redundant database as well as select thermal metagenomes from the Joint Genome Institute Integrated Microbial Genome (JGI IMG) database Table 2. We sought to limit the search to thermostable enzymes capable of PET hydrolysis near the PET Tg. To this end, we leveraged the correlation between enzyme maximum temperatures and the optimal growth temperature (OGT) of the host organisms. Hence, the HMM sequence hits were mapped to OGT data retrieved from the NCBI Bioproject database, the BacDive database, and the JGI IMG metagenome sample temperature. Sequences with OGT lower than 50° C. were discarded. For sequences that could not be mapped to OGT data, we trained a ML model (ThermoProt) to discriminate between 8,000 proteins from thermophiles (>50° C.) and 8,000 proteins from non-thermophiles (<50° C.) using the support vector machine method with calculated amino acid features. ThermoProt demonstrated an accuracy of 86.6% in five-fold cross-validation tests.

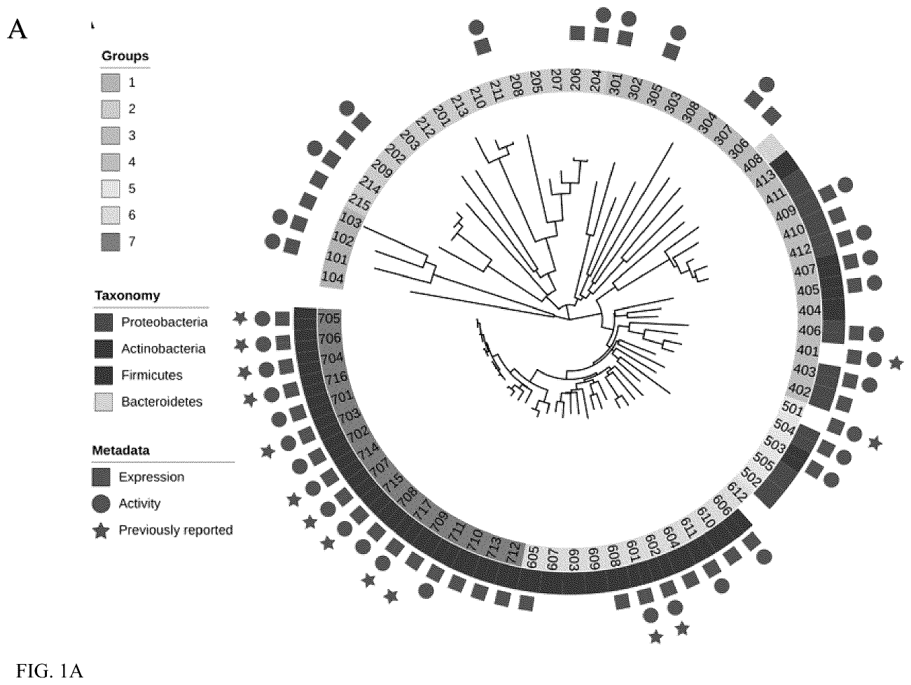

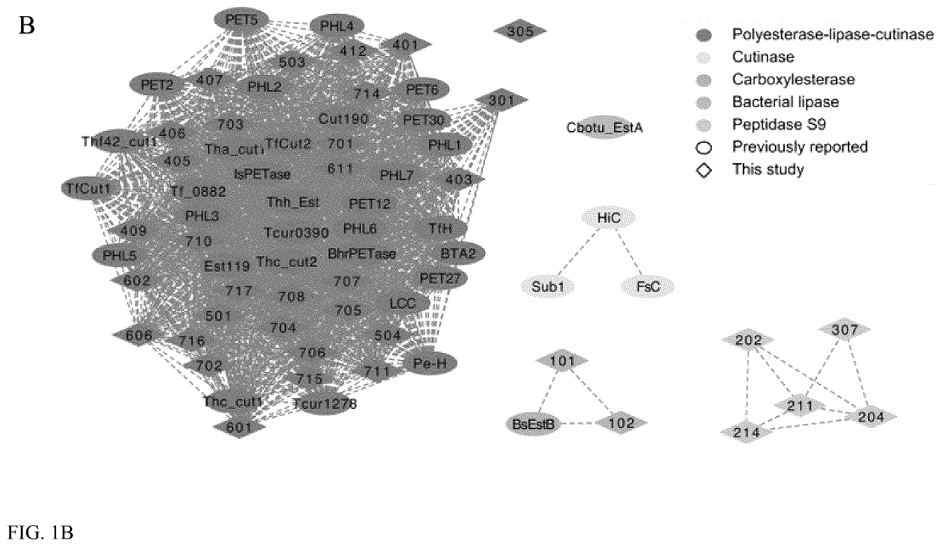

We observed that many of the top HMM hits from the JGI IMG metagenomes were identical or very similar to hits from NCBI. To diversify the sequence search space further, we selected proteins with predicted thermostability and high HMM scores (>100, E-value<8.0e⁻²⁶) from the NCBI hits, but thermophile-derived proteins with relatively low scores (<55, E-value>2.0e⁻¹¹) from the JGI IMG hits. Consequently, 74 sequences were selected. We note that 14 of these sequences have been reported in other studies to our knowledge and were retained in our assays as benchmarks. As illustrated in FIG. 1A, phylogenetic analysis showed that these 74 sequences comprise at least seven distinct phylogenetic groups, with the diverse JGI IMG sequences forming three clades (which we termed groups 1 to 3) that are clearly separate from the NCBI sequences. The NCBI sequences form two clades (which we termed groups 6 and 7) and two paraphyletic groups (termed groups 4 and 5) (FIG. 1A). Based on these results, the 74 PET hydrolase candidate sequences were assigned identification numbers according to these phylogenetic groups (101 and 102 in group 1, 201 and 202 in group 2, and so on). A list of candidate sequences is provided in an annotated description with accession numbers for each in Table 3.

To gain insight into the diversity of the selected sequences within the vast α/β hydrolase superfamily, we classified the sequences according to families in the ESTHER database (56) and predicted enzyme commission (EC) numbers. EC number predictions were assigned by transferring EC numbers (1) associated with the ESTHER families, (2) associated with the top annotated hit from a BLAST search of each sequence against the SwissProt database, and (3) predicted by the deep-learning tool, DeepEC. The results reveal that all candidate sequences in groups 4 to 7 with high HMM scores (>100) belong to the polyesterase-lipase-cutinase family, along with nearly all previously reported PET hydrolases, and are associated with carboxyl ester hydrolase (3.1.1.-) and cutinase (3.1.1.74) activities. However, the sequences derived from lower HMM scores (groups 1 to 3) diverge from canonical PET hydrolases and are associated with distant families such as peptidases E.C. (3.4.-.-). A sequence similarity network (FIG. 1B) demonstrates the clustering of currently known PET hydrolases in the polyesterase-lipase-cutinase family and the divergence of candidate sequences from groups 1 to 3.

Screening on amorphous PET shows that PET hydrolysis activity is distributed among all seven phylogenetic groups. The 74 enzymes were expressed in Escherichia coli with each putative PET hydrolase gene codon-optimized and cloned into a pET21b(+) plasmid with a C-terminal hexa-histidine epitope tag. The likelihood of a signal peptide sequence in each of the 74 putative enzyme sequences was predicted using SignalP 5.0, and the resulting predictions were removed in the 36 relevant expression constructs (vide infra). Given the diversity of enzymes to be expressed and purified, we adopted a 4-stage expression screening approach that varied E. coli expression strains, growth medium composition, incubation temperature and time, induction protocol, and other relevant expression parameters. Enzyme purification followed a standardized protocol of affinity chromatography, buffer exchange, and size exclusion chromatography, Table 4 details the expression strategies that enabled production of 51 of the 74 enzymes.

Given the possible range of enzyme activities, we employed a comprehensive, semi-quantitative screening assay to first detect PET hydrolytic activity of each enzyme. Specifically, we used 100 mM NaCl with 50 mM buffer across a range of pH (citrate at pH 6.0, NaH₂PO₄ at pH 7.0, NaH₂PO₄ at pH 7.5, HEPES at pH 7.5, bicine at pH 8.0, and glycine at pH 9.0) and temperature (30° C. to 70° C., in 10° C. increments). All screening reactions were conducted in triplicate. In this initial activity screen, we employed commercially available amorphous PET film from Goodfellow, thereby enabling inter-study comparisons. All reactions were conducted for 96 h at an enzyme loading of 0.7 mg enzyme/g PET and a substrate loading of 2.9% by mass in polypropylene microcentrifuge tubes. Due to the molecular weight differences of the enzymes screened, the number of catalytic units added to the reactions differed. However, we chose this approach given that enzyme loadings for reactions of this nature are typically assessed for process cost on the basis of mass of enzyme loaded per mass of substrate. The aromatic reaction products, bis(2-hydroxyethyl) terephthalate (BHET), mono(2-hydroxyethyl) terephthalate (MHET), and TPA, were quantitated via ultra-high-performance liquid chromatography up to a product concentration of 500 mg/L accounting for dilution, above which the calibration curve was outside of the linear range. For this substrate loading, the upper limit of concentration of product corresponds to a maximum extent of conversion of 2.1% by mass. Aromatic product release data are reported throughout, relative to background aromatic product release detected in no-enzyme control reactions at each pH and temperature. As positive controls, we included the LCC wild-type enzyme and two improved mutant variants (ICCG and WCCG), the I. sakaiensis PETase wild-type enzyme and an improved double mutant variant (W159H/S238F), and T. fusca cutinase BTA-1.

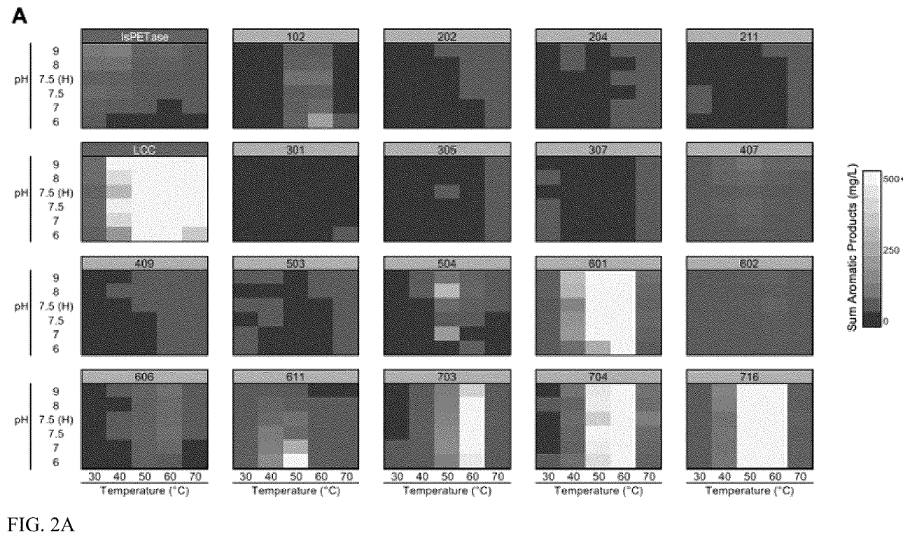

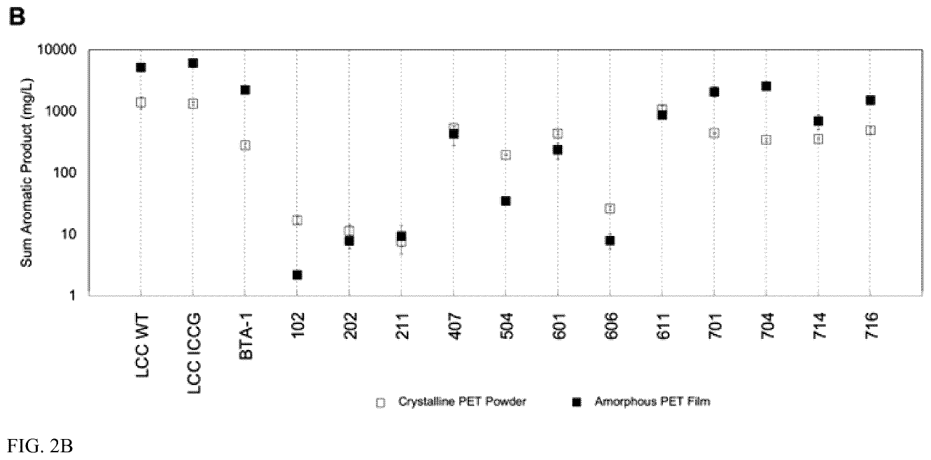

FIG. 2A shows illustrative heat maps of total aromatic product release across 30 reaction conditions for the best-performing enzymes from each of the seven phylogenetic groups, alongside two positive control enzymes, namely wild-type LCC and I. sakaiensis PETase. At least one enzyme from each of the phylogenetic groups shown in FIG. 1 exhibited measurable PET hydrolysis activity. Overall, 36 enzymes were found to be active for PET hydrolysis at statistically significant levels above the no-enzyme control, while 14 of the 51 enzymes did not exhibit any detectable PET hydrolytic activity above the no-enzyme control background. FIG. 2A shows that enzymes in groups 5, 6, and 7 exhibited the highest detected activity. This is not surprising given that most of the enzyme discovery efforts to date on PET hydrolases have identified enzymes belonging to the polyesterase-lipase-cutinase family, to which the enzymes in groups 5, 6, and 7 belong. Groups 1 and 4 also exhibited appreciable PET hydrolysis activity, while groups 2 and 3 displayed only minimal activity above the no-enzyme control background. Overall, this screening highlights 23 thermostable enzymes that have not been previously reported, to our knowledge and that exhibit PET hydrolase activity beyond the 36 currently known enzymes.

As is apparent in FIG. 2A, there is a substantial breadth of enzyme activity across the pH and temperature ranges studied, with activity of at least one enzyme in every condition tested. For the four enzymes that exhibited optimal activity at pH 6.0 (102, 611, 702, 715), we further extended the pH screen across the same five temperatures and four additional pH conditions (50 mM citrate buffer at pH 5.0 and 5.5 and 50 mM sodium acetate buffer at pH 4.5 and 5.0), with the LCC wild-type enzyme and the LCC ICCG mutant as positive controls. The LCC ICCG mutant is active in buffered medium with a pH as low as 5.0, while 102 was not active in media with a pH of less than 6.0, and 611, 702, and 715 all exhibit detectable activity in medium with a pH less than 6.0.

Lastly, because I. sakaiensis PETase and some cutinases are secreted 34e, we were interested in the potential effects on both protein expression and hydrolytic activity when signal peptide sequences predicted to enable protein secretion were included. We conducted the same screening experiments for a selection of putative PET hydrolases retaining the native signal peptide (nSP) in the expression sequence, namely 301, 401, 403, 410, 606, 607, and 711. The results demonstrate that the inclusion of a signal peptide in the expression sequence does not uniformly influence activity, as illustrated by our observations of complete abolishment of activity (301, 410, 711), a slight increase in activity (606), and reduction of activity (401, 403). Enzyme 607 could only be expressed when including the native signal peptide sequence, though much of the enzyme produced is insoluble. Enzyme 607-nSP (with native peptide) exhibited measurable PET hydrolytic activity, increasing the total number of unique catalytic domains expressed and screened to 52, and the number of new, thermostable PET hydrolases identified to 24.

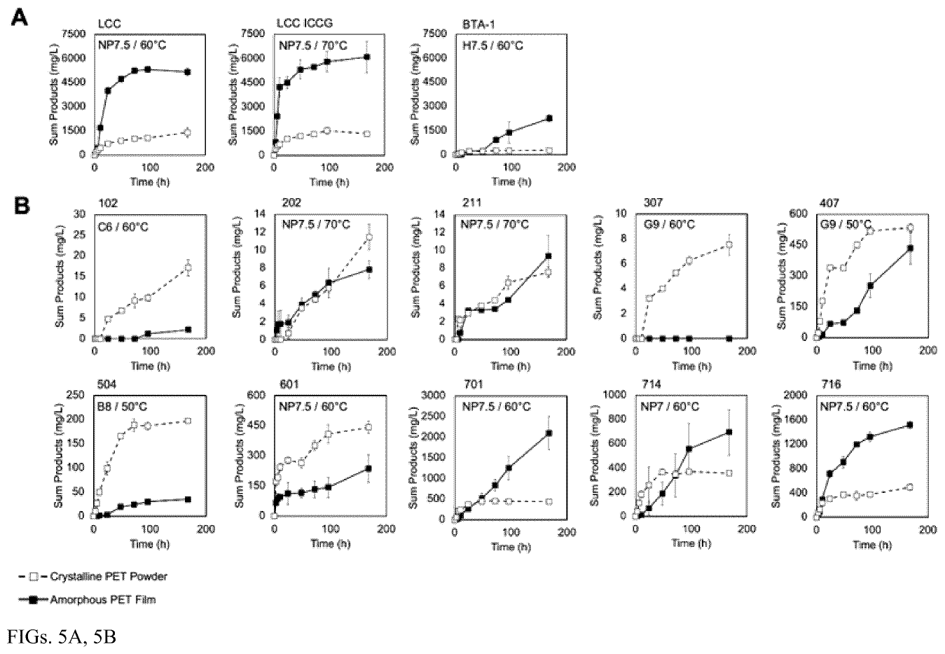

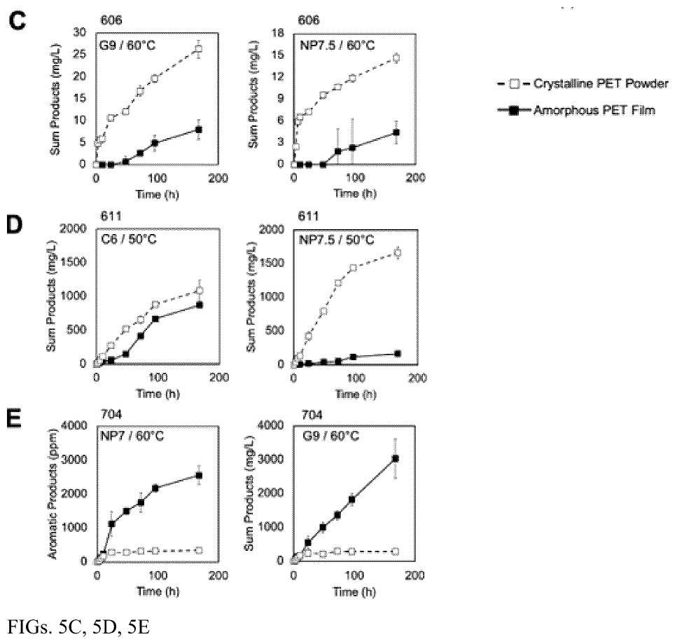

Detailed characterization of the best-performing enzymes highlights reactivity differences on different substrates. We were also interested to learn if the best-performing enzymes from each phylogenetic group would exhibit different reactivity profiles on different PET substrates. For these comparisons we used two commercially available substrates that have been thoroughly characterized, namely a crystalline Goodfellow PET powder and a Goodfellow amorphous PET film. This set included 12 enzymes selected to represent a diverse group with the highest PET degradation extents observed from screening, see FIG. 2B and FIG. 5. Experiments were conducted with the LCC wild-type enzyme, the LCC ICCG mutant, and BTA-1 as positive controls. The reactions were run for 168 h to compare effects due to enzyme stability. As shown in FIG. 2B, both control enzymes and a several group 7 enzymes (701, 704, 714, 716) exhibited higher activity on amorphous PET film, consistent with prior work. However, we also identified enzymes with higher activity on crystalline PET powder compared to amorphous PET film (FIG. 2B), which has not previously been reported for thermophilic PET hydrolases to our knowledge. Additional comparisons of the 168 h reactions are in FIG. 5. Table 5 depicts the corresponding reaction conditions employed in these experiments and the data.

Calorimetry confirms thermostability across the phylogenetic groups. Of the expressed and purified enzymes, 20 were of sufficient yield and solubility for thermostability analysis by differential scanning calorimetry (DSC), including at least one member from each of the seven distinct phylogenetic groups. The observed melting temperature (Tm) values in neutral buffer for the 17 enzymes of known origin (belonging to groups 4-7) ranged from 53.9° C. for enzyme 606 originating from Marinactinospora thermotolerans, to 86.9° C. for wild-type LCC (501), see Table 6. In addition, Tm values were obtained for single representative members from groups 1-3, each of which originates from metagenomic sequences from environmental samples. Two of these, enzymes 102) (66.0° C. and 202 (75.1° C.), have Tm values within the established range for known thermophilic enzymes, whilst enzyme 306 exhibited the highest Tm (92.6° C.) of all 20 enzymes analyzed. These measurements confirm the utility of the Thermoplot ML algorithm in identifying amino acid sequences with high thermal stability.

The majority of the above enzymes that were amenable to DSC analysis are members of group 7, including eight highly homologous polyester-lipase-cutinase enzymes originating from T. fusca (701-706, 714 and 715), and three from T. cellulosylitica (709, 711 and 716). With the exception of 709, each of these exhibit some degree of PET hydrolase activity. This comprehensive T. fusca enzyme DSC dataset illustrates the potential variation in thermostability (65.6 to 71.8° C.) for homologous secreted enzymes from a single thermophilic species; from a biological perspective, such variation is tolerable since, in all cases, the Tm exceeds the OGT of the organism. An analysis of the Tm sequence dependence in these enzymes reveals point variants that influence their thermostability; for example, enzymes 702 and 705, which are 99% identical in sequence and differ at only three amino acid positions, have Tm values separated by 6.2° C. Such differences in their susceptibility to thermal denaturation may influence the optimal temperatures for PET hydrolysis and inform further engineering.

Structural characterization highlights diversity of PET-active enzymes. Given the range of sequence diversity captured in this work (FIG. 1B) and the opportunities to interrogate structure-function relationships across a broad group, we conducted comprehensive crystallization screening, resulting in eight high-resolution X-ray structures for enzymes 202 (7QJM), 306 (7QJN), 606 (7QJO), 611 (7QJP), 702 (7QJQ), 703 (7QJR), 705 (7QJS), and 711 (7QJT) at resolutions extending between 1.43-2.19 Å. As observed previously, the compact folds of α/β hydrolases can often yield high-quality atomic, and even sub-atomic, resolution X-ray data. However, as we screened beyond the folds homologous to the I. sakaiensis, Thermobifida, and LCC enzymes, the success rate of crystallization hits fell. With PET-active representatives identified in all seven phylogenetic groups, we sought to use the AlphaFold protein structure prediction system to interrogate the structural diversity of the 74 enzymes.

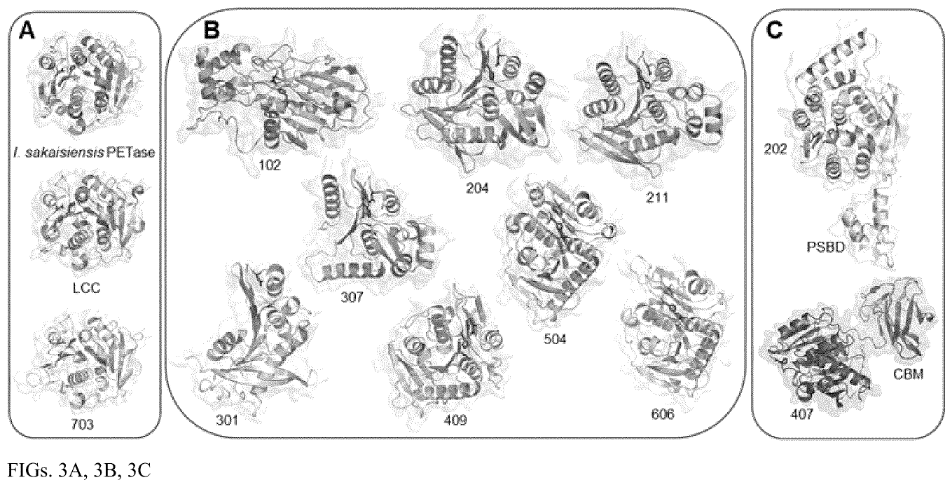

To investigate the utility of AlphaFold for thermotolerant enzyme folds, we first selected sequences where we already had unpublished X-ray structures, allowing direct comparison between the predictions and experimental data. In line with recent observations on compact folds within the human proteome, we observed that pLDDT data, the AlphaFold quality scoring metric (a per-residue measure of local confidence on a scale from 0-100 based on a Local Distance Difference Test), were generally favorable, indicating high confidence in the accuracy of these target structures. Superposition with the experimental structures revealed a high correlation with the general architecture, and geometric predictions matched the experimental structures down to the level of individual residues. This was particularly the case for residues that form key structural interactions within the core of the proteins and, crucially, those contributing to the active sites. Further validation of the utility of this approach was demonstrated by the successful use of an AlphaFold structure as a molecular replacement search model for a challenging experimental X-ray dataset from enzyme 306. Based on these results, we used AlphaFold to predict all 74 structures, with a selection of PET-active enzymes shown in FIG. 3.

As shown in FIG. 3A, representatives of known PET hydrolase enzymes, such as those in groups 5-7, share highly similar structures. Here, we show that expanded primary sequence phylogeny correlates with an unexpectedly large increase in structural diversity, not simply changes in surface loops and secondary structural elements, but large core deletions, modifications, and substantial fold extensions and additions (FIG. 3B). Overall, this group of enzymes spans molecular weights ranging from 13 to 55 kDa (I. sakaiensis PETase is ˜27 kDa) and isoelectric points from 4.3 to 9.7, see Table 3. We focus on examples that capture the range of diversity, describing enzymes that are active on PET, and present structural features not previously associated with PET hydrolysis. Using LCC as the archetypal comparator, we explore multiple levels of structural divergence, from subtle changes in the catalytic cleft and surface charge distribution, to additional domains, major core deletions, and new folds constituting alternative active site arrangements and binding modes.

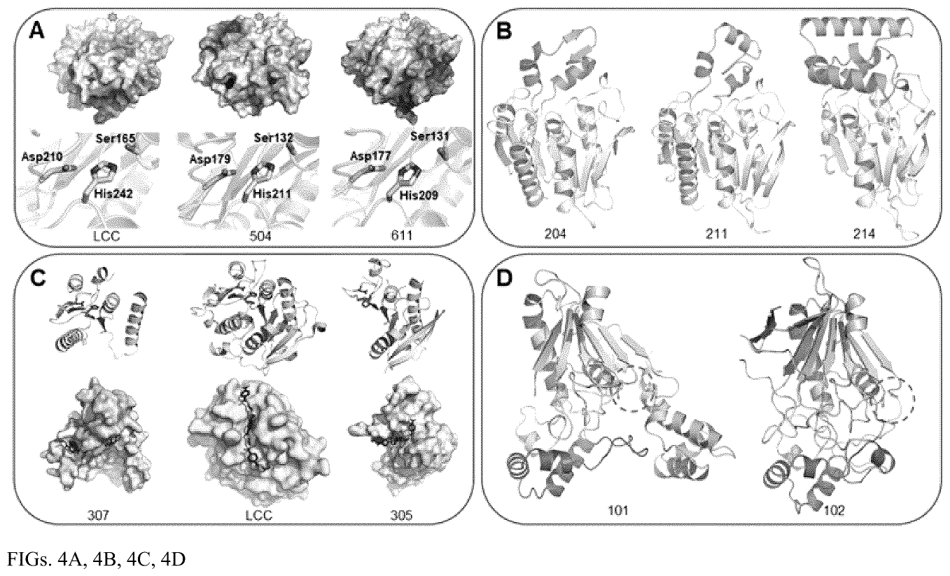

Wide ranging surface residue modifications provide functional diversity while maintaining a conserved catalytic core. The group 5, 6, and 7 enzymes are the most characterized to date and share many common features including a highly conserved core domain with a 9-stranded B-sheet flanked by 8 or 9 α-helices. While the newly identified candidates in this study have not yet been subjected to protein engineering, these groups represent generally the most active members of the cohort of 74. Given their close similarities and the wealth of structural data, we were curious if there was a structural rationale for the observed differences in substrate preference in groups 5 and 6 compared to LCC, which itself is in group 5 (FIG. 2B). A comparison of LCC with enzymes 504 and 611 reveals high similarities, with RMSDs of 0.92 Å over 1,361 atoms and 0.81 Å over 1,366 atoms, respectively. With an X-ray structure of enzyme 611 extending to 1.56 Å, and a high-confidence AlphaFold model of enzyme 504, comparisons revealed almost identical active site triad geometries (FIG. 4A) making the substrate crystallinity differences surprising.

To investigate this further, analysis of the surface charge distribution revealed a highly acidic patch adjacent to the active site cavity of enzyme 504 compared to LCC, while 611 displays an exceptionally acidic surface extending around multiple faces, in stark contrast to canonical PET hydrolases that are generally more positively charged on the solvent-exposed surface (FIG. 4A). This correlates with an isoelectric point of 4.3 for enzyme 611, compared to 9.3 and 9.5 for LCC and the I. sakaiensis PETase, respectively.

A closer look at the active sites of 504 and 611 reveals more subtle, but potentially key differences. We employed computational substrate docking to compare the relative active site surface cavities and their influence on substrate binding (SI Appendix, FIG. S9). While LCC accommodates a PET trimer deep within a cleft, resulting in significant twisting of the aromatic molecules in the polymer chain, enzymes 504 and 611 present shallow clefts that appear to bind the polymer chain in a straighter conformation, possibly playing a role in the preferential accommodation of crystalline rather than amorphous PET observed as disclosed herein.

Evolution of multiple lid and accessory domains generate additional variety. A variety of accessory domains is observed in groups 2, 3 and 4, ranging from small lids that cap or partially occlude the predicted active site regions, to large independent folds connected by flexible linkers (FIG. 3C, 4B). These include a Peripheral Subunit-Binding Domain (PSBD) in enzyme 202, not initially observed in the X-ray crystal structure, but revealed by AlphaFold predictions, and a Family 35 carbohydrate binding module (CBM) in enzyme 407 (FIG. 3C). Perhaps unsurprisingly, two candidates from the set of 74 enzymes that were not successfully expressed in E. coli included enzyme 408, which contains a putative cell wall anchor domain, and enzyme 212, which contains a predicted extended transmembrane anchor.

The group 2 enzymes represent a new family of peptidase-like hydrolases, all characterized by a central core with the addition of lid domains in a variety of constructions. Examples include a mixed helical and B-sheet arrangement (204), a three-helix bundle (211), and for enzyme 214, a substantial 80-residue extended helical domain which creates a 40 Å wide flat surface platform of unknown function, see FIG. 4B.

It is of note that the shapes of the group 2 active site clefts are also unusual. For example, the active site is partially covered in enzyme 204. However, this region of the predicted structure has a low confidence score in the AlphaFold prediction and may be dynamic. Nevertheless, equivalent elements are well defined in the X-ray structure of enzyme 202 to a resolution of 2.19 Å, a particularly interesting candidate given that it has a Tm of 75° C. It is similar to enzyme 214 in term of the extensive lid domain, but enzyme 202 has two large α-helices and two B-strands which substantially extend the central B-sheet. Combined with the attachment of the PSBD, this is the largest of representative of the Group 2 enzymes with a molecular mass of 41.5 kDa. In a departure from classical PET hydrolases, the active site is completely buried in this apo crystal structure, and while the two occluding structures, a helix on one side and a loop on the other, look to be robustly linked by hydrogen bonds and hydrophobic stacking interactions, these two regions have the highest B-factors of the catalytic core. In fact, the occluding helix sits on what appears to be a hinge-like structure which may have the potential to swing open to accommodate the polymer chain. If this was to occur, the cavity would expose 3 aromatic phenylalanine residues toward the PET surface.

Mini-PETases reconstitute productive active sites from only half the core domain. Enzyme 307 has a large deletion of around one half of the core domain, with only four strands in the central B-sheet compared to the typical eight or more strands found in canonical PET hydrolases, see FIG. 4C. Enzyme 307 would be the smallest protein in the set of 74, if not for the addition of a compact active site lid. Despite the absence of four helices in the core, this enzyme remarkably retains the conserved canonical active site. As a result of the deletion, the 307 active site is open in nature and docking studies predict potential electrostatic interactions that may stabilize an otherwise flexible protein following substrate binding. Docking simulations with a PET trimer reveal the potential for binding within a large open cleft, as compared to the relatively narrow groove of the LCC active site FIG. 4C. The same minimal fold is also observed in candidate 201, in this case without the lid domain, making it the smallest representative from the entire set at 15.6 kDa. While not expressed in sufficient quantities for biochemical analysis, given it has the same active site triad arrangement, it may still find productive use for modelling the absolute minimal scaffold solution for a 4 β-stranded PET hydrolase.

Highlighting the differences within a single phylogenetic group, enzyme 305 also displays a major deletion, but more surprisingly in the opposite half of the core compared to 307. The missing a-helical region would normally contribute half of the active site cavity and the His residue of the active site triad in the canonical fold. On closer inspection, an alternative His is positioned in the triad, reconstituting what appears to be a unique active site from the same half of the core. Both of these mini-PETases offer opportunities to investigate the minimal protein chain required for PET hydrolysis via two alternative active sites and may provide a starting point for de novo protein design.

Newly identified PET-active family members offer alternative folds, binding surfaces, and active site geometries. While the group 1 enzymes exhibit low activity relative to the other groups, examples such as enzyme 102 with a Tm of 65° C., are quite thermotolerant. These enzymes exhibit a distinct fold, closer to carboxylesterases, such as the EST55 enzyme from Geobacillus stearothermophilus (PDB ID 20GT), see FIG. 4D, and a previously identified mesophilic enzyme with PET activity, Bacillus subtilis p-nitrobenzylesterase, BsEstB. An Alphafold structural model reveals that the BsEstB enzyme is similar to EST55, sharing the same 3-domain architecture (catalytic, regulatory, and α/B) with conserved active site triad residues. However, the PET-active group 1 enzymes from this study are structurally divergent from these examples. For example, enzymes 101 and 102 have comparatively large deletions in the main catalytic domain, and enzyme 102 lacks the regulatory domain entirely (FIG. 4D). These truncations are significant because in the canonical fold they contribute around one half of the active site environment, including the catalytic His and Glu residues. Both 101 and 102 conserve the position of the catalytic Ser, but there is no equivalently positioned His in 101, and no equivalently positioned His or Glu in 102. Further studies will be required to characterize the active sites in these enzymes where major domain deletions result in unusually large flat surfaces surrounding potential active sites.

### Discussion

Enzymes capable of PET hydrolysis have been sourced thus far from a relatively narrow sequence space, and therefore unlikely fully encompass the natural diversity that can catalyze this reaction. Using bioinformatics and ML to gather sequences from environmental and cultivar genomes, we have discovered several distinct enzymes that hydrolyze PET, likely all via a serine hydrolase mechanism based on conservation of the catalytic triad, but with different enzyme architectures. We observed multiple adaptations in this enzyme cohort that will benefit from more detailed study. Many of these rearrangements and adaptations create alternative active site clefts, gorges, and planes, which may provide a useful diversity of structural motifs to achieve efficient interfacial biocatalysis for PET deconstruction. Furthermore, distinct differences in surface charge and in binding mode provide tractable parameters for enzyme engineering to develop biocatalysts with high selectivity for crystalline PET substrates. There are also many subtler adaptations observed in these enzymes, such as diverse N-glycosylation site distributions, which has previously been shown to confer significant reduction in thermal induced aggregation. Deletion and complementation of accessory domains could also provide productive improvement in enzyme performance. For example, several of the group 2 lid domains have N- and C-terminal attachment points in close proximity that could be trimmed, removed, or swapped to test the effects on active site occlusion and substrate binding. These data also indicate that signal peptide sequences, when present in the native genes, should be considered in the screening of putative PET hydrolases.

It is likely that lessons from canonical PET hydrolases will be more challenging to directly transfer to the enzymes from groups 1-3. Nevertheless, even for those enzymes with marginal activity on PET, the structural and biophysical characteristics provide a foothold for pursuing enzyme evolution. Improvement of these enzymes will benefit from the continuing advances in high-throughput screening and selection techniques. Again, this structural diversity combined with varied functional properties, including a range of thermal stabilities, pH operating ranges, and substrate discrimination, will provide new starting points for parallel engineering projects using these new folds. With the advent of enhanced structural predictions such as AlphaFold and RoseTTAFold, not only can we quickly gain structural insights from our most promising candidates, but we also gain additional insights from those enzyme homologs that are inactive. These technologies will allow the productive combination of negative and positive data to provide richer input for further engineering.

This disclosure herein should enable the discovery of additional enzyme scaffolds in nature. The JGI IMG sequences in groups 1 to 3 yielded low alignment scores with the PET hydrolase HMM (Table 3), and several of these sequences showed hydrolytic activity on PET, despite being markedly diverse relative to canonical PET hydrolases. This finding suggests that the distribution of currently known PET hydrolases, which are largely limited to the polyesterase-lipase-cutinase family (FIG. 1B), may result from biases of sequence similarity and HMM methods that limit the search to a narrow sequence space within the vicinity of canonical PET-active enzyme. To this end, our data points present a wider diversity of PET hydrolases across environmental gradients, and which should be the targets of continued exploration.

To provide insight into the governing sequence characteristics responsible for PET hydrolysis, we further examined the ability of HMM scores to discriminate between active PET hydrolases and inactive homologs by computing the area under the curve (AUC) of the receiver operating characteristic plot and the Spearman correlation coefficient (p) between HMM scores and our experimental activity data. Our results indicate that the HMM scores demonstrate mediocre performance in predicting PET hydrolase activity of putative hits (AUC=0.581, p=0.167). Furthermore, we investigated the distribution of amino acids at each position in a multiple sequence alignment (MSA) of active PET hydrolases and inactive homologs to identify positions that correlate with activity and, therefore, could play key roles in PET hydrolysis activity. However, we did not find statistically significant (p<0.01) relationships between positional variation in the MSA and activity. This suggests that pairwise covariation and higher-order interactions that are not captured by the HMM play dominant roles in PET hydrolase activity. Recent studies have shown that ML can successfully capture such complex pairwise interactions. Consequently, the application of ML with our experimental activity data within a semi-supervised framework provides promise for improved prospecting of additional active PET hydrolases.

Given the diversity of putative PET hydrolases studied here, there was a risk of missing active enzymes by relying upon a limited range of expression conditions and activity assays. To mitigate this, we considered a range of heterologous protein expression and reaction conditions. Fortunately, some enzymes were active across broad temperature and pH ranges, while others exhibited narrower windows for activity. The screening results also highlight challenges associated with direct comparison of enzymes, where peak product release may be comparable, but the reaction conditions affording that are not. Furthermore, we found that codon optimization leads to substantially different expression and activity levels with different extents of codon optimization, including for the LCC enzyme and the corresponding 501 enzyme, and BTA-1 and 715, enzyme pairs with identical protein sequences but different nucleotide sequences. Another critical consideration in identifying additional PET-active enzymes are the PET substrate properties. We screened for activity using an amorphous PET film, and yet, upon further characterization, we observed selectivity differences for amorphous PET relative to a crystalline PET powder. This suggests screening should also be conducted using diverse substrates, in addition to multiple reaction conditions. While 74 enzymes represent only a modest number relative to variant libraries commonly encountered in enzyme evolution, we anticipate the lessons learned here will inform future screening efforts.

Our analysis of candidates from this study already extends to some industrially relevant functional parameters. For example, multiple studies have shown that high substrate crystallinity leads to reduced conversion extents relative to amorphous PET. From an industrial perspective, this has led to an emphasis on substrate pretreatment to thermo-mechanically convert post-consumer PET waste to an amorphous substrate. We recently reported a techno-economic analysis and life cycle assessment of enzymatic PET recycling. Of direct relevance to PET crystallinity and pretreatment, the base case process model included thermal extrusion, rapid quenching, and mechanical size reduction via a microgranulator to reduce the crystallinity of PET from post-consumer PET flake. Sensitivity analysis indicates a potential reduction in process electricity usage by 67%, overall process energy reductions of nearly 50%, and a savings of $0.24/kg recovered TPA if extensive substrate pretreatment could be avoided, thus motivating an interest in enzymes with specificity to crystalline substrates. As shown in FIG. 2B and FIG. 3, 102, 504, 611, and several other enzymes preferentially deconstruct crystalline PET powder relative to amorphous PET film, which suggests exciting possibilities in biocatalyst development for crystalline PET. For example, these enzymes could be used as a foundation from which to develop improved variants that retain preferential selectivity on crystalline PET, or defining differentiating enzyme features, such as surface charge distribution or binding clefts shape. Such features could be transplanted to the best-performing amorphous-active enzymes to assess potential gain-of-function on crystalline substrates. Moreover, this also suggests the potential to develop cocktails of PET hydrolases that contain enzymes with synergistic substrate specificity for amorphous and crystalline domains in the substrate, similar to how cellulase cocktails deconstruct cellulose. This could ultimately enable new avenues to enable enzymatic hydrolysis on PET waste with reduced pretreatment energy inputs.

### Materials and Methods

### Sequence Search and Alignments

Environmental metagenomes (n=3,136) were retrieved from the Joint Genome Institute Integrated Microbial Genome (JGI IMG) database in April 2017. The metagenomes were first categorized into sub-categories (thermal springs, groundwater) as previously reported, and only thermal spring metagenomes were considered further (Table 2). Sequences from these metagenomes were retrieved (˜38 million sequences). The National Center for Biotechnology Information (NCBI) non-redundant database was also downloaded as of 20 Dec. 2018 (˜184 million sequences). A dataset of 17 enzymes that have been confirmed to exhibit PET hydrolysis activity as of 20 Dec. 2018 was compiled (Table 1). Sequences of the 17 PETases were retrieved and aligned with T-Coffee. T-Coffee performed better in aligning the distantly related sequences, compared to MAFFT, ClustalW2, and MUSCLE, particularly in correct placement of the catalytic Ser and His residues and the terminal Cys residues.

A profile hidden Markov Model (HMM) was constructed with the PETase alignment using the HMMER software (version 3.1b2) and putative PET hydrolases were retrieved by hmmsearch of the HMM against the retrieved NCBI and JGI IMG sequences. The NCBI search returned 2,165 hits with alignment scores ranging from 100 to 442 (E-value: 7.7e⁻²⁵ to 8.6e⁻¹²⁹). To diversify the sequence search space, the HMM threshold was lowered for the JGI IMG search and sequences with relatively lower scores were selected. The JGI search returned 1,367 hits with alignment scores ranging from 26 to 360 (E-value: 1.0e⁻² to 1.8e⁻¹⁰⁴). For organisms from which the NCBI sequence hits were derived, optimal growth temperature (OGT) data were retrieved from the NCBI Bioproject database (https://www.ncbi.nlm.nih.gov/bioproject/) and the BacDive database (10) (https://bacdive.dsmz.de/). The sample temperatures of the JGI IMG metagenomes (Table S2) were used as the OGT for the JGI IMG sequence hits. To limit the search to thermostable sequences, only thermophilic sequences with OGT of 50° C. or greater were selected. Among the NCBI hits, 31 were selected as thermophilic, 1,777 were mesophilic and were discarded, and 353 were from organisms that could not be mapped to OGT data. The thermophilicity of these sequences that could not be mapped to OGT data was predicted with ThermoProt (vide infra). The final selection included 58 thermophilic sequences (predicted/OGT) from NCBI (scores: 104-442, E-values: 8.0e⁻²⁶-8.6e⁻¹²⁹) and 35 sequences from JGI IMG (scores: 27-35, E-values: 3.0e⁻³-2.6e⁻⁵). Redundant sequences (100% identity, excluding the predicted signal peptide region) were removed, which left 74 putative thermophilic PET hydrolases in the selection (Table 3).

Unless otherwise stated, structure-based multiple sequence alignments were used in all further analyses. The structure-based alignment was performed as follows. First, a structural alignment of all crystal structures and AlphaFold structure models presented in this work was performed with the Promals3D web server. Then, all sequences to be analyzed were aligned with MAFFT using the structural alignment as constraint. Sequence analyses were implemented with the Biopython package.

Prediction of Thermophilicity with Machine Learning (ThermoProt)

From the NCBI and BacDive databases, sequence and OGT data were retrieved for 24 organisms classified as psychrophilic (<15° C.), mesophilic (25-37° C.), thermophilic (45-) 70° C., or hyperthermophilic (>80° C.). A separate testing set was formed of 22,299 proteins from an organism in each OGT class, and the remaining sequences (231,171) were used in training and validation. To prevent overestimation of the validation performance, the sequences were clustered at 40% sequence-identity threshold using the CD-HIT algorithm. From the CD-HIT output, 40,000 sequences were selected for validation such that there were 10,000 sequences in each class, with 8,000 sequences (2,000 in each class) set aside for hyperparameter optimization and feature selection, while the remaining 32,000 (8,000 in each class) were used for training, validation, and analysis.

Three categories of features were derived from the protein sequences.

Amino acid composition features: the relative amounts of 20 canonical amino acids in the sequence.

g-gap dipeptide composition: the relative amounts of the peptide, a(x)gb, where a and b are specific amino acids and (x)g represents g amino acids of any type, sandwiched between a and b. In this work, 1,200 g-gap dipeptides (i.e., g=0, 1, and 2) were tested and the top 10 were selected by their relative (Gini) importance in a random forest model. Additional g-gap dipeptides beyond 10 did not improve the random-forest classification performance.

Residue type and physiochemical features: in addition, 20 features that have been shown in previous studies to correlate with thermal stability were selected, namely the composition of acidic, basic, non-polar, acyclic, aliphatic, aromatic, charged, and EFMR (Glu, Phe, Met, Arg) residues; the ratio of basic to acidic, non-polar to polar, acyclic to cyclic, and charged to non-charged residues; the composition of tiny (Ala, Gly, Pro, Ser) and small (Thr, Asp) residues, the average maximum solvent accessible area (ASA), the ratio of (Glu+Lys) to (Gln+His), charged vs. polar composition (18), IVYWREL (Ile, Val, Tyr, Trp, Arg, Glu, Leu) composition, molecular weight, and heat capacity.

Five machine-learning methods were tested with the Scikit-learn Python package (21): random forests, logistic regression, Gaussian naïve Bayes, K-nearest neighbor, and support vector machine (SVM). Hyperparameters for each method were optimized with a grid search using dataset of 8,000 proteins (2,000 per class). Four binary classifiers were tested: psychrophilic vs. mesophilic (PM), mesophilic vs. thermophilic (MT), thermophilic vs. hyperthermophilic (TH), and mesophilic vs. thermophilic/hyperthermophilic (MTH). Machine-learning methods with the different binary classification schemes were used and measured over fivefold cross-validation with the dataset of 32,000 proteins (8,000 per class). All methods achieve accuracies between 68.0% and 86.6%. In addition to the accuracy, the true positive rate (recall), true negative rate (specificity), and Matthew's correlation coefficient were also computed. The SVM method (termed ThermoProt) yielded the best performance (MTH, 86.6% accuracy) and was applied to the PETase HMM hits without OGT data to predict the thermophilicity.

It is important to note that while this work was ongoing, a dataset of OGT for 21,498 microbes was published which enabled regression models that directly predict the OGT (23, 24), and the optimal catalytic temperature (Topt) of an enzyme. These regression methods could be applied in future works for more precise prediction of the thermotolerance of putative PETases.

Discrimination of Active PETases from Inactive Homologs with Hidden Markov Models (HMM).

Sequence data of 60 enzymes with experimentally confirmed PET hydrolase activity were compiled, comprising 36 PETases reported in other studies (Table S1) and 24 non-redundant PETases newly presented in this study. Sequence data of 19 homologs that are experimentally confirmed to be inactive on PET were also compiled, comprising 15 sequences from this study, and PET28, PET29, PET38 (26), and Cbotu_EstB reported previously. A structure-based alignment of all 79 active and inactive sequences was performed, and the alignment was split to separate sub-alignment of active and inactive sequences.

The performance of HMM in discriminating active PETases from inactive homologs was evaluated with fivefold cross-validation. The active/inactive sequences were split into five folds and the HMM was repeatedly built with the data in four folds and evaluated with the data in the left-out fold such that each fold was iteratively used in training and testing. Two methods of HMM prediction were considered. First, an HMM was built with active PETases in the training set and searched against sequences in the testing set. The HMM alignment score of test sequences was construed as a predictive measure of PET hydrolase activity (score method). In the second method (difference method), an additional HMM was built with inactive homologs in the training set, and searched against the testing set. The difference between the HMM score obtained from the active PETase HMM and the score from the inactive homologs HMM was construed as the predictive measure of PET hydrolase activity. With the score method, it is expected that sequences exhibiting high PET hydrolase activity would have high scores when searched against an HMM of active PETases, while inactive sequences or sequences with low activity would have low scores. With the difference method, it is expected that active sequences would have higher scores when searched against an HMM of active PETases than when searched against an HMM of inactive homologs, and, consequently, a higher score difference. Similar HMM approaches have proven remarkably successful in discriminating functional subtypes in protein families. However, the results indicate that HMM only demonstrates mediocre performance in discriminating PETases from inactive homologs.

In addition, the amino-acid distribution in the alignment of active PET hydrolases and inactive homologs was investigated. If a residue position plays key roles in activity, it is expected that the amino acid distribution at that position would significantly vary between actives and inactives. A chi-squared test of independence was performed to compare the amino-acid distribution at each position in the structure-based alignment between 60 active PETases and 19 inactive homologs. Positions with gaps in more than 90% of the sequences were removed (805 removed, 437 remaining). The test was also performed to compare the distribution of amino acid types (aliphatic: Ala, Gly, Val, Leu, Ile, Met, Cys, Pro; aromatic: Phe, Trp, Tyr, His; positive: Arg, Lys; negative: Asp, Glu; polar: Asn, Gln, Ser, Thr). The results indicate that no single position in the alignment shows statistically significant difference (p<0.01) between active PETase and inactive homologs.

### Phylogenetic Analyses and Sequence Similarity Network

Phylogenetic analyses were conducted with the MEGAX software. For the phylogeny of 74 candidate sequences (FIG. 1A), the evolutionary history was inferred using the Minimum Evolution (ME) method. The evolutionary distances were computed using the JTT matrix-based model and are in the units of the number of amino acid substitutions per site. The ME tree was searched using the Close-Neighbor-Interchange (CNI) algorithm at a search level of 1. The Neighbor-joining algorithm was used to generate the initial tree. All ambiguous positions were removed for each sequence pair with the pairwise deletion option.

A separate tree was constructed to further illustrate the phylogenetic relationships of 36 previously reported PET-hydrolases and the unique PET-hydrolases presented in this study using the maximum likelihood method with 1000 replicates and the JTT matrix-based model. The initial tree for the heuristic search was obtained by applying the Neighbor-Join and BioNJ algorithms to a matrix of pairwise distances estimated using the JTT model, and then selecting the topology with superior log likelihood value. All positions with less than 95% site coverage were eliminated. The phylogenetic trees were visualized with the Interactive Tree of Life (iTOL) online tool.

The sequence similarity network (SSN) (FIG. 1B, main text) was implemented with the Enzyme Function Initiative Enzyme Similarity Tool (EFI-EST). Sequences were subjected to a BLASTall pairwise search and the SSN was constructed with a threshold of 1e⁻¹⁰. The SSN was visualized with Cytoscape.

### Materials

Amorphous PET film (Product ES301445) and crystalline PET powder (Product 306031) were purchased from Goodfellow Corporation (USA). Percent crystallinity was for each substrate has previously been reported. All reagents and buffer components were acquired from Sigma-Aldrich.

### Plasmid Construction

Coding sequences were codon optimized for Escherichia coli str. K-12 MG1655 using a guided random approach from the OPTIMIZER server (http://genomes.urv.es/OPTIMIZER). Optimized sequences for expression of the 6 control hydrolases (wild-type IsPETase, mutant variant IsPETase (W159H/S238F), wild-type LCC, the ICCG variant of LCC, the WCCG variant of LCC, and BTA-1), and all versions of the 74 candidate enzymes were synthesized by Twist Biosciences in pET21b(+) (EMD Millipore)-based plasmids. Each construct includes a C-terminal hexa-histidine epitope tag. Sequences are provided in Table SD1 (candidates) and Table SD2 (controls). All 74 genetic expression constructs have been deposited at AddGene at https://www.addgene.org/Gregg_Beckham/.

### Enzyme Expression

For identifying soluble heterologous protein expression, BL21 (DE3) E. coli (NEB), OverExpress™ C41 (DE3) (Lucigen), and Lemo21 (DE3) (NEB) competent cells were used. Competent cells were transformed with pET21b(+) plasmids encoding the enzyme of interest. Single colonies from transformation were then inoculated into a starter culture of lysogeny broth (LB) media containing 100 μg/mL ampicillin and grown at 37° C. overnight. Four expression strategies were evaluated using 50 mL cultures and soluble expression was evaluated by SDS-PAGE with Coomassie staining and Western blot using primary antibody against the hexa-histidine epitope tag (Invitrogen). Using results from the 50 mL scale expression tests, the best condition was chosen for each control or candidate and scaled to 1-5 L, depending on expression level. Table S10 details which competent cell line and expression strategy was used for each control and candidate enzyme, and the final expression level (mg enzyme/L culture) obtained for each enzyme.

In strategy A, the starter culture was inoculated at a 100-fold dilution into a 2×YT medium (10 g NaCl, 10 g yeast extract, 16 g tryptone per L culture) containing 100 μg/mL ampicillin and grown at 37° C. until the optical density measured at 600 nm (OD600) reached 0.6-0.8. Protein expression was then induced by addition of isopropyl β-D-1-thiogalactopyranoside (IPTG) to a final concentration of 1 mM. Cells were induced at 20° C. for 18 to 24 h following IPTG addition, harvested by centrifugation, and stored at −80° C. until purification.

In strategy B, the starter culture was inoculated at a 100-fold dilution into a 2×YT medium containing 100 μg/mL ampicillin and grown at 37° C. until the OD600 reached 0.6. Protein expression was then induced by addition of IPTG to a final concentration of 0.5 mM. Cells were induced at 25° C. for 16 to 18 h following IPTG addition, harvested by centrifugation, and stored at −80° C. until purification.

In strategy C, the starter culture was inoculated at a 1000-fold dilution into ZYP-5052 medium containing 100 μg/mL ampicillin and grown at 28° C. for 24 h. Cells were harvested by centrifugation and stored at −80° C. until purification.

In strategy D, the starter culture was inoculated at a 500-fold dilution into ZYP-5052 medium with 0.3 M NaCl containing 100 μg/mL ampicillin and grown at 25° C. for 72 h. Cells were harvested by centrifugation and stored at −80° C. until purification.

### Enzyme Purification

Harvested cells were thawed on ice and resuspended in a lysis buffer (300 mM NaCl, 10 mM imidazole, 20 mM Tris HCl, pH 8.0,) with 0.25 mg/mL lysozyme, and 12.5 U/mL DNase I. Cells were lysed using either a bead beater (BioSpec Products, Inc.) or sonication with a microtip (39% power, 20 s ON, 20 s OFF for a total of 2 min 20 s ON). Lysate was clarified by centrifugation at 40,000×g for 40 minutes at 4° C. Clarified lysate was filtered through a 0.45 μm PVDF membrane, then applied to a 5 mL HisTrap HP (Cytiva) affinity column using an ÄKTA Pure chromatography system (Cytiva) and eluted using a buffer comprising 300 mM NaCl, 500 mM imidazole, 20 mM Tris HCl, pH 8.0. Resulting fractions containing the protein of interest were pooled and dialyzed at room temperature (25° C.) using 3.5 kDa molecular weight exclusion membranes in an exchange reservoir at least 300 times the pooled sample volume of 300 mM NaCl, 20 mM Tris, pH 8.0 buffer. After 16 to 20 h of buffer exchange, samples were centrifuged and evaluated by SDS-PAGE with Coomassie staining. Pooled samples were concentrated using 3.5 kDa molecular weight cut-off spin columns and applied to a HiLoad Superdex 75 pg 16/60 (Cytiva) size exclusion column equilibrated with 300 mM NaCl, 20 mM Tris, pH 8.0 for use in screening or time course analysis. Protein in eluted fractions from affinity and size exclusion columns were assessed using SDS-PAGE with Coomassie staining and Western blot using primary antibody against the hexa-histidine epitope tag (Invitrogen). Total protein was assessed by BCA assay.

### Signal Peptide Sequences

Presence of signal peptide sequences was predicted using SignalP 5.0 (40). From 74 putative thermophilic PET hydrolase sequences, 36 signal peptides were removed for construct synthesis. A selection of 12 truncated constructs that proved challenging to express were re-synthesized to include the native signal peptide (nSP) and compared for changes in expression and activity. Of these signal peptide-containing constructs, 7 were successfully expressed and screened, of which, only 607 could not be expressed without the native signal peptide. Sequences for the nSP-containing candidates are provided in Table SD1. Additionally, expression of the Thh_Est enzyme (710) was previously reported from an expression plasmid (pET26b(+)) containing an N-terminal pelB signal peptide. Both the truncated version of 710 and the pelB-containing version (710-pelB) expressed enzyme, but neither showed activity during screening (data not shown for 710-pelB).

### Protein Calorimetry (DSC)

Apparent melting temperature (Tm) values for those purified enzymes that were sufficiently soluble (>0.1 mg/mL) in neutral buffer were assessed by differential scanning calorimetry (DSC). Immediately prior to DSC analysis, to ensure both mono-dispersity and an optimal buffer match, each enzyme was prepared by size-exclusion chromatography (SEC) through a HiLoad Superdex 75 pg column (Cytiva) pre-equilibrated with the DSC reference buffer comprising 50 mM NaH₂PO₄, pH 7.5, with either 300 mM NaCl (for 606) or 100 mM NaCl (for all other enzymes). The SEC column was calibrated with a mixture of globular protein standards (Sigma-Aldrich)-thyroglobulin (670 kDa), γ-globulin (158 kDa), albumin (67.0 kDa) and ribonuclease A (13.7 kDa)—to allow for the calculation of an apparent molecular weight (MWapp) for each enzyme from its elution volume. Subsequently, triplicate DSC analyses, each using 0.1-0.2 mg/mL enzyme, were performed on a MicroCal PEAQ-DSC-Automated instrument (Malvern Panalytical). The temperature of the sample and reference cells was raised from 30° C. to 120° C. at a rate of 1.5° C./min using low feedback. Thereafter, reference buffer subtraction, baseline correction and apparent Tm determination were performed using the instrument's data analysis software (v1.60).

### Monomer Quantitation

Analyte analysis of BHET, MHET, and TPA was performed on an Infinity II 1290 ultra-high-performance liquid chromatography (UHPLC) system (Agilent Technologies) equipped with a G7117A diode array detector (DAD). Samples and standards were injected using a volume of 0.25 μL onto a Zorbax Eclipse Plus C18 Rapid Resolution HD (2.1×50 mm, 1.8 μm) (Agilent Technologies) column maintained at 40° C. The mobile phase used to separate the analytes of interest was composed of (A) 20 mM phosphoric acid in ultrapure water and (B) 100% methanol. Separation of analytes was carried out using a constant flow rate of 0.7 mL/min and a gradient program with a total run time of 3 min. The gradient program proceeded as follows: at t=0 min, (A)=80% and (B)=20%; at t=2 min, (A)=35% and (B)=65%; from t=2.01 min until the end at t=3 min, (A)=80% and (B)=20%. The calibration curve for each analyte was evaluated between concentrations of 1-200 mg/L with DAD detection at a wavelength of 240 nm. Ten calibration standards were used with an R2 coefficient of 0.995 or better. Calibration verification standards (CVS) for each analyte was analyzed every 12-24 samples to ensure the integrity of the initial calibration. Samples were diluted with ultrapure water for analysis and maintained at 15° C. during the analysis.

### Screening for Activity on Amorphous PET Film

In each screening reaction, 2.9% loading by mass of an amorphous PET film (Goodfellow) was incubated with 10 μg enzyme of interest (0.7 mg enzyme/g PET), unless noted otherwise in Table 4 due to low expression levels. Reactions were performed in polypropylene tubes containing 100 mM NaCl and 50 mM buffering agent (citrate at pH 6.0, NaH2PO4 at pH 7.0, NaH2PO4 at pH 7.5, HEPES at pH 7.5, bicine at pH 8.0, and glycine at pH 9.0) and incubated at 30° C., 40° C., 50° C., 60° C., or 70° C. All reactions were terminated after 96 h by addition of equal volume 100% methanol and PET was removed from the reaction solution. Soluble fractions were filtered through 0.2 μm nylon filters for monomer quantitation. All PET hydrolysis screening reactions were performed in triplicate.

For enzymes with peak activity at pH 6.0, an extended pH screening assay was performed using 2.9% loading by mass of amorphous PET film (Goodfellow) and 10 μg enzyme of interest (0.7 mg enzyme/g PET enzyme loading) in polypropylene tubes containing 100 mM NaCl and 50 mM citrate (pH 5.5 and pH 5.0) or 50 mM sodium acetate (pH 5.0 and pH 4.5). All reactions were terminated after 96 h by addition of equal volume 100% methanol and PET was removed from the reaction solution. Soluble fractions were filtered through 0.2 μm nylon filters for monomer quantitation. All PET hydrolysis screening reactions were performed in triplicate.

Aromatic product release data are reported throughout relative to background aromatic product release detected in no-enzyme control reactions at each pH and temperature. Background aromatic product release for both amorphous PET film and crystalline PET powder was below the detection limit for all pH and temperature combinations tested.

Characterization of PET Hydrolysis Activity on Varied Substrates with Time Resolution

Using the reaction conditions (buffer and temperature combination) where peak PET hydrolysis activity was measured from the screening assays, a selection of enzymes was further characterized over a 168 h reaction on amorphous PET film (Goodfellow) and crystalline PET powder (Goodfellow) substrates. Each reaction was performed using 2.9% by mass substrate loading and 10 μg enzyme of interest (0.7 mg enzyme/g PET). Reactions were terminated at the designated timepoint by addition of equal volume 100% methanol and PET was removed from the reaction solution. Soluble fractions were filtered through 0.2 μm nylon filters for monomer quantitation. All time course experiments were performed in triplicate and samples were diluted with ultrapure water for analyte quantitation. Table 5 provides details on the enzyme and reaction condition pairings evaluated over 168 h reaction time.

### Structure Determination

For crystallography, all proteins were concentrated and sitting drop crystallization trials were set up with a Mosquito crystallization robot (SPT Labtech) using SWISSCI 3-lens low profile crystallization plates. The proteins were crystallized using the following screens and conditions:
202—JCSG-plus screen (Molecular Dimensions), G7, 15% PEG 3350, 0.1 M succinic acid.
306—SaltRx screen (Hampton Research), E8, 1.8 M sodium phosphate monobasic monohydrate, potassium phosphate dibasic pH 5.0.
606—Structure screen (Molecular Dimensions), F5, 0.1 M Sodium HEPES pH 7.5, 70% (v/v) MPD.
611—PACT screen (Molecular Dimensions), F1, 20% PEG 3350, 0.2 M sodium fluoride, 0.1 M Bis-Tris propane pH 6.5.
702—PACT screen (Molecular Dimensions), F8, 20% PEG 3350, 0.2 M sodium sulfate, 0.1 M Bis-Tris propane pH 6.5.
703—PACT screen (Molecular Dimensions), F10, 20% PEG 3350, 0.02 M sodium/potassium phosphate, 0.1 M Bis-Tris propane pH 6.5.
705—JCSG screen (Molecular Dimensions), F1, 0.05 M Cesium Chloride, 0.1 M MES pH 6.5, 30% (v/v) Jeffamine M-600.
711—JCSG screen (Molecular Dimensions), D6, 0.2 M Magnesium Chloride Hexahydrate, 0.1 M Tris pH 8.5, 20% (w/v) PEG 8000.

All crystals were cryo-protected with 20% glycerol in the crystallization solution and flash-frozen into liquid nitrogen. Diffraction data were collected at the Diamond Light Source (Didcot, UK) and automatically processed with STARANISO on ISPyB. STARANISO was also used for processing anisotropic data and calculating ellipsoidal completeness. The structure was solved within CCP4 Cloud by molecular replacement with Molrep (2) using search models created by phyre2. For 306, MR was solved with an AlphaFold structure prediction. Model buildings were performed in Coot and the structures were refined with BUSTER and REFMAC5. MolProbity was used to evaluate the final models and PyMOL (Schrödinger, LLC) for protein model visualizations. The atomic coordinates have been deposited in the Protein Data Bank. Search for structural protein homologs and calculation of RMSD values were performed with the DALI server.

AlphaFold structure predictions were generated using the same models and inference procedure as employed in CASP14. This is described in the recent AlphaFold paper. Mean pLDDT (predicted local distance difference test) over the structure was used for model ranking, and pLDDT values were written into the B-factor column of each structure file.

### Molecular Docking

Molecular docking calculations were performed using the program Molecular Operating Environment (MOE). Flexible PET dimers and trimers were optimized inside a rigid host structure. Initial placement of the PET oligomer units was carried out using the Triangle Matcher approach, with subsequent refinement via molecular mechanics. The position and energy of 200 poses were optimized and their ranking was carried out based on the highest molecular mechanics interaction energy, E_refine.

TABLE 1
List of current experimentally verified PET hydrolases. The HMM column
shows the 17 sequences used in constructing the HMM, which were among
the PET hydrolases known at the time of the initial enzyme candidate
selection. The Candidate Enzyme ID column shows the identifier for sequences
that are also contained in our set of 74 putative PET hydrolases.

| | | | | | Candidate |
|---|---|---|---|---|---|
| | Organism | Name | Accession | HMM | Enzyme ID |
| 1 | Ideonella sarkaiensis | IsPETase | GAP38373.1 | 1 | |
| 2 | Thermobifida fusca | BTA-1 (TfH, | WP_011291330.1 | 2 | 715 |
| | DSM43793 | Tfu_0883, | | | |
| | | Cut2) | | | |
| 3 | Uncultured bacterium | LCC | AEV21261 | 3 | 501 |
| 4 | Fusarium solani pisi | FsC | 1CEX_A | 4 | |
| 5 | Thermobifida | Thc_cut1 | ADV92526.1 | 5 | |
| | cellulosilytica | | | | |
| | DSM44535 | | | | |
| 6 | Thermobifida | Thc_cut2 | ADV92527.1 | 6 | 716 (DM) |
| | cellulosilytica | | | | |
| | DSM44535 | | | | |
| 7 | Thermobifida fusca | Thf42_cut1 | ADV92528.1 | 7 | 703 |
| | DSM44342 | | | | |
| 8 | Thermobifida alba | Tha_cut1 | ADV92525.1 | 8 | 707 |
| 9 | Thermobifida | Thh_Est | AFA45122.1 | 9 | 710 |
| | halotolerans DSM44931 | | | | |
| 10 | Sachharomonospora | Cut190 | BAO42836.1 | 10 | |
| | viridus AHK190 | | | | |
| 11 | Humicola insolens | HiC | 4OYY_A | 11 | |
| 12 | Bacillus subtilis | BsEstB | ADH43200.1 | 12 | |
| 13 | Thermonospora curvata | Tcur1278 | CDN67545.1 | 13 | 601 |
| | DSM43183 | | | | |
| 14 | Uncultured bacterium | PET2 | ACC95208.1 | 14 | 401 |
| | | (lipIAF5-2) | | | |
| 15 | Oleispira antartica RB-8 | PET5 (lipA) | CCK74972.1 | 15 | |
| 16 | Vibrio gazogenes | PET6 | WP_021018894.1 | 16 | |
| 17 | Polyangium | PET12 | WP_047194864.1 | 17 | |
| | brachysporum | (AAW51_2473) | | | |
| 18 | Thermonospora curvata | Tcur0390 | CDN67546.1 | | 602 |
| | DSM43183 | | | | |
| 19 | Thermobifida fusca KW3 | TfCut1 | CBY05529.1 | | 704 |
| 20 | Thermobifida fusca | BTA2 | CAH17554.1 | | 706 |
| 21 | Thermobifida fusca KW3 | TfCut2 | CBY05530.1 | | 714 |
| 22 | Thermobifida fusca YX | Tf_0882 | AAZ54920.1 | | 705 |
| | | (Cut1) | | | |
| 23 | Streptomyces scabiei | Sub1 | QEX94755.1 | | |
| 24 | Clostridium botulinum | Cbotu_EstA | AKZ20828.1 | | |
| | ATCC3502 | | | | |
| 25 | Bacterium HR29 | BhrPETase | GBD22443.1 | | |
| 26 | Pseudomonas aestusnigri | Pe-H | 6SBN_A | | |
| 27 | Aequorivita sp. | PET27 | WP_111881932.1 | | |
| | CIP111184 | | | | |
| 28 | Chryseobacterium | PET30 | WP_039353427.1 | | |
| | (Kaistella) jeonii | | | | |
| 29 | Compost metagenome | PHL1 | LT571440 | | |
| 30 | Compost metagenome | PHL2 | LT571441 | | |
| 31 | Compost metagenome | PHL3 | LT571442 | | |
| 32 | Compost metagenome | PHL4 | LT571443 | | |
| 33 | Compost metagenome | PHL5 | LT571444 | | |
| 34 | Compost metagenome | PHL6 | LT571445 | | |
| 35 | Compost metagenome | PHL7 | LT571446 | | |
| 36 | Thermobifida alba | Est119 (Est2) | BAK48590.1 | | 717 |
| | AHK119 | | | | |

TABLE 2
JGI IMG metagenomes from which putative sequences were derived. These metagenomes comprised
a total of 38 million sequences, which were searched against the PETase HMM to derive putative
PET hydrolases. The rows that are bolded in the Scaffold Key column highlight metagenomes
from which the JGI candidates in our dataset (27 out of 74) were derived.

| | | | | Sample | |
|---|---|---|---|---|---|
| | | | | Temp./ | |
| | | Gold | | Ecosystem | |
| Scaffold | IMG | Ecosystem | Geographic | Subtype | Sample |
| Key | Genome ID | Type | Location | (° C.) | pH |
| Deep | 3300001781 | Marine | Cayman Islands, UK | — | — |
| Ga0063234 | 3300005209 | Thermal springs | Yellowstone National Park, | 42.0-90.0 | — |
| | | | USA | | |
| Ga0063235 | 3300004269 | Thermal springs | Yellowstone National Park, | 42.0-90.0 | |
| | | | USA | | |
| Ga0073359 | 3300005292 | Thermal springs | Yellowstone National Park, | 42.0-90.0 | |
| | | | USA | | |
| Ga0073360 | 3300005291 | Thermal springs | Yellowstone National Park, | 42.0-90.0 | |
| | | | USA | | |
| Ga0073929 | 3300007070 | Thermal springs | British Columbia, Canada | 66.4 | 7.93 |
| Ga0073930 | 3300007071 | Thermal springs | British Columbia, Canada | 64.7 | 7.94 |
| Ga0073931 | 3300006951 | Thermal springs | British Columbia, Canada | 85.9 | 7.08 |
| Ga0073932 | 3300007072 | Thermal springs | British Columbia, Canada | 64.7 | 7.94 |
| Ga0073933 | 3300006945 | Thermal springs | British Columbia, Canada | 44.5 | 8.15 |
| Ga0073934 | 3300006865 | Thermal springs | British Columbia, Canada | 33.1 | 7.16 |
| Ga0074394 | 3300005396 | Thermal springs | Yellowstone National Park, | 42.0-90.0 | — |
| | | | USA | | |
| Ga0079041 | 3300006857 | Thermal springs | Yellowstone National Park, | 42.0-90.0 | — |
| | | | USA | | |
| Ga0079042 | 3300006181 | Thermal springs | Yellowstone National Park, | 42.0-90.0 | — |
| | | | USA | | |
| Ga0079043 | 3300006179 | Thermal springs | Yellowstone National Park, | 42.0-90.0 | — |
| | | | USA | | |
| Ga0079044 | 3300006855 | Thermal springs | Yellowstone National Park, | 42.0-90.0 | — |
| | | | USA | | |
| Ga0079046 | 3300006859 | Thermal springs | Yellowstone National Park, | 42.0-90.0 | — |
| | | | USA | | |
| Ga0079048 | 3300006858 | Thermal springs | Yellowstone National Park, | 42.0-90.0 | — |
| | | | USA | | |
| Ga0105154 | 3300009598 | Thermal springs | Sandy's Spring West, Nevada, | 86.6 | 7.03 |
| | | | USA | | |
| Ga0105155 | 3300009591 | Thermal springs | Sandy's Spring West, Nevada, | 86.6 | 7.03 |
| | | | USA | | |
| Ga0105156 | 3300009596 | Thermal springs | Sandy's Spring West, Nevada, | 86.6 | 7.03 |
| | | | USA | | |
| Ga0105158 | 3300008019 | Thermal springs | Little Hot Creek, California, | 81.1 | 6.83 |
| | | | USA | | |
| Ga0105159 | 3300009590 | Thermal springs | Little Hot Creek, California, | 81.1 | 6.83 |
| | | | USA | | |
| Ga0105160 | 3300009585 | Thermal springs | Gongxiaoshe Hot Spring,, | 73.8 | 7.29 |
| | | | China | | |
| Ga0105161 | 3300009013 | Thermal springs | Gongxiaoshe Hot Spring,, | 71.7 | 7.46 |
| | | | China | | |
| Ga0105162 | 3300008000 | Thermal springs | Baoshan, Yunnan, China | 78.2 | 6.65 |
| Ga0105163 | 3300007999 | Thermal springs | Baoshan, Yunnan, China | 81.6 | 6.71 |
| Ga0114943 | 3300009626 | Thermal springs | Beatty, Nevada, USA | 42.0-90.0 | — |
| Ga0114944 | 3300009691 | Thermal springs | Beatty, Nevada, USA | 42.0-90.0 | — |
| Ga0114945 | 3300009444 | Thermal springs | Beatty, Nevada, USA | 42.0-90.0 | — |
| Ga0116196 | 3300010393 | Thermal springs | Zodletone Spring, Oklahoma, | 10.0 | 7.50 |
| | | | USA | | |
| Ga0116197 | 3300010317 | Thermal springs | Zodletone Spring, Oklahoma, | 10.0 | 7.50 |
| | | | USA | | |
| Ga0116210 | 3300010288 | Thermal springs | Tshipise, South Africa | 42.0-90.0 | — |
| Ga0116211 | 3300010313 | Thermal springs | Limpopo, South Africa | 42.0-90.0 | — |
| Ga0123519 | 3300009503 | Thermal springs | Yellowstone National Park, | 42.0-90.0 | — |
| | | | USA | | |
| Ga0129299 | 3300010289 | Thermal springs | California, USA | 45.6 | 8.08 |
| Ga0129301 | 3300010284 | Thermal springs | California, USA | 45.6 | 8.08 |
| Ga0129302 | 3300010291 | Thermal springs | California, USA | 42.0-90.0 | 7.48 |
| Ga0137047 | 3300010484 | Thermal springs | British Columbia, Canada | 85.9 | 7.08 |
| Ga0137159 | 3300010494 | Thermal springs | British Columbia, Canada | 85.9 | 7.08 |
| Ga0137169 | 3300010514 | Thermal springs | British Columbia, Canada | 85.9 | 7.08 |
| Ga0137224 | 3300010600 | Thermal springs | British Columbia, Canada | 85.9 | |
| Ga0137240 | 3300010575 | Thermal springs | British Columbia, Canada | 85.9 | 7.08 |
| Ga0167615 | 3300013009 | Thermal springs | Yellowstone National Park, | 68.0 | 3.00 |
| | | | USA | | |
| Ga0167616 | 3300013008 | Thermal springs | Yellowstone National Park, | 78.0 | 3.00 |
| | | | USA | | |
| Ga0170330 | 3300013082 | Thermal springs | British Columbia, Canada | 85.9 | 7.08 |
| Ga0170563 | 3300013084 | Thermal springs | British Columbia, Canada | 85.9 | 7.08 |
| Ga0170564 | 3300013085 | Thermal springs | British Columbia, Canada | 85.9 | 7.08 |
| GxsBSedJan11 | 3300000865 | Thermal springs | Gongxiaoshe pool, Tengchong, | 73.8 | 7.29 |
| | | | China | | |
| JGI20127J14776 | 3300001382 | Thermal springs | Yellowstone National Park, | 42.0-90.0 | — |
| | | | USA | | |
| JGI20128J18817 | 3300001684 | Non-marine | Yellowstone National Park, | — | — |
| | | saline and | USA | | |
| | | alkaline | | | |
| JGI20132J14458 | 3300001339 | Thermal springs | Yellowstone National Park, | 83.0 | 8.60 |
| | | | USA | | |
| JGI24227J36426 | 3300002555 | Thermal springs | Yellowstone National Park, | 42.0-90.0 | — |
| | | | USA | | |
| JGI24228J36427 | 3300002539 | Thermal springs | Yellowstone National Park, | 42.0-90.0 | — |
| | | | USA | | |
| JGI24229J36425 | 3300002556 | Thermal springs | Yellowstone National Park, | 42.0-90.0 | — |
| | | | USA | | |
| JGI24230J36428 | 3300002540 | Thermal springs | Yellowstone National Park, | 42.0-90.0 | — |
| | | | USA | | |
| JGI24231J26847 | 3300002208 | Thermal springs | Yellowstone National Park, | 42.0-90.0 | — |
| | | | USA | | |
| JGI24717J26846 | 3300002207 | Thermal springs | Yellowstone National Park, | 42.0-90.0 | — |
| | | | USA | | |
| JGI24718J22297 | 3300001986 | Thermal springs | Yellowstone National Park, | 42.0-90.0 | — |
| | | | USA | | |
| JGI24721J26819 | 3300002182 | Thermal springs | Yellowstone National Park, | 42.0-90.0 | — |
| | | | USA | | |
| JGI24721J44947 | 3300005573 | Thermal springs | Yellowstone National Park, | 42.0-90.0 | — |
| | | | USA | | |
| JGI26464J51801 | 3300003604 | Thermal springs | Yellowstone National Park, | 42.0-90.0 | — |
| | | | USA | | |
| JGI26465J51735 | 3300003598 | Thermal springs | Yellowstone National Park, | 42.0-90.0 | — |
| | | | USA | | |
| JGI26466J51736 | 3300003603 | Thermal springs | Yellowstone National Park, | — | — |
| | | | USA | | |
| JGIcombinedJ22296 | 3300001987 | Thermal springs | Yellowstone National Park, | 42.0-90.0 | — |
| | | | USA | | |
| JzSedJan11 | 3300000866 | Thermal springs | Baoshan, Yunnan, China | 81.6 | 6.71 |
| shallow | 3300001835 | Marine | Cayman Islands, UK | — | — |
| YNP11 | 2014031007 | Thermal springs | Yellowstone National Park, | 82.0 | 7.90 |
| | | | USA | | |
| YNP15294550 | 2015219002 | Thermal springs | Yellowstone National Park, | 59.9 | 8.20 |
| | | | USA | | |
| YNP15490790 | 2015219002 | Thermal springs | Yellowstone National Park, | 59.9 | 8.20 |
| | | | USA | | |
| YNP16 | 2016842003 | Thermal springs | Yellowstone National Park, | 36.0 | 9.10 |
| | | | USA | | |
| YNP17 | 2016842005 | Thermal springs | Yellowstone National Park, | 56.0 | 5.70 |
| | | | USA | | |
| YNP18 | 2016842004 | Thermal springs | Yellowstone National Park, | 76.0 | 6.40 |
| | | | USA | | |
| YNP20 | 2016842008 | Thermal springs | Yellowstone National Park, | 52.0 | 6.30 |
| | | | USA | | |
| YNP3 | 2014031003 | Thermal springs | Yellowstone National Park, | 80.0 | 4.00 |
| | | | USA | | |
| YNP3A | 2016842001 | Thermal springs | Yellowstone National Park, | 80.0 | 4.00 |
| | | | USA | | |
| YNP6 | 2013515000 | Thermal springs | Yellowstone National Park, | 50.0 | — |
| | | | USA | | |
| YNP7 | 2014031006 | Thermal springs | Yellowstone National Park, | 52.9 | 6.00 |
| | | | USA | | |
| YNPsite05 | 2022920003 | Thermal springs | Yellowstone National Park, | 57.6 | 6.20 |
| | | | USA | | |
| YNPsite06 | 2022920004 | Thermal springs | Yellowstone National Park, | 50.0 | — |
| | | | USA | | |
| YNPsite07 | 2022920013 | Thermal springs | Yellowstone National Park, | 52.9 | 6.00 |
| | | | USA | | |
| YNPsite11 | 2022920012 | Thermal springs | Yellowstone National Park, | 82.0 | 7.90 |
| | | | USA | | |
| YNPsite15 | 2022920016 | Thermal springs | Yellowstone National Park, | 59.9 | 8.20 |
| | | | USA | | |
| YNPsite16 | 2022920018 | Thermal springs | Yellowstone National Park, | 36.0/ | 9.10 |
| | | | USA | (42.0-90.0) | |
| YNPsite17 | 2022920021 | Thermal springs | Yellowstone National Park, | 56.0 | 5.70 |
| | | | USA | | |
| YNPsite18 | 2022920019 | Thermal springs | Yellowstone National Park, | 76.0 | 6.40 |
| | | | USA | | |
| YNPsite20 | 2022920020 | Thermal springs | Yellowstone National Park, | 52.0 | 6.20 |
| | | | USA | | |

TABLE 3
Annotated list of the 74 candidate enzymes. The HMM score column shows the alignment scores obtained
by searching the HMM built with 17 experimentally confirmed PETases against the NCBI and JGI databases.
Sequences in groups 1 to 3 were retrieved from JGI IMG and the accession column shows the scaffold
ID mapping the sequence to the corresponding metagenome (see Table 2). Sequences in groups 4 to
7 were retrieved from NCBI and the accession column shows the GenBank accession number.

| | | | | | | | Predicted |
|---|---|---|---|---|---|---|---|
| | | | | | | | molecular |
| | | | | | | | weight |
| | | Enzyme | | | HMM | Theoretical | (w/o His |
| | Group | ID | Accession/ID | Organism | score | pI | tag) |
| 1 | 1 | 101 | YNPsite06_CeleraDRAFT_263770 | Environmental sample | 34.6 | 7.10 | 32.2 |
| 2 | | 102 | YNP6_02150 | Environmental sample | 35.1 | 5.42 | 31.0 |
| 3 | | 103 | GxsBSedJan11_10003667 | Environmental sample | 35.3 | 6.49 | 55.0 |
| 4 | | 104 | YNP16_304900 | Environmental sample | 30.8 | 4.97 | 41.0 |
| 5 | 2 | 201 | YNP15490790 | Environmental sample | 28.9 | 5.08 | 15.6 |
| 6 | | 202 | YNPsite05_CeleraDRAFT_401410 | Environmental sample | 30.3 | 6.03 | 41.5 |
| 7 | | 203 | YNP16_189140 | Environmental sample | 27.5 | 9.47 | 21.6 |
| 8 | | 204 | YNP18_240440 | Environmental sample | 40.5 | 6.07 | 27.0 |
| 9 | | 205 | JzSedJan11_10146151 | Environmental sample | 45.4 | 5.99 | 22.2 |
| 10 | | 206 | JGI20127J14776_10147151 | Environmental sample | 37.8 | 6.33 | 27.0 |
| 11 | | 207 | YNPsite18_CeleraDRAFT_262380 | Environmental sample | 45.8 | 6.91 | 24.0 |
| 12 | | 208 | JzSedJan11_10131225 | Environmental sample | 37.6 | 6.51 | 29.0 |
| 13 | | 209 | YNPsite20_CeleraDRAFT_325860 | Environmental sample | 29.8 | 5.77 | 37.4 |
| 14 | | 210 | JzSedJan11_10073025 | Environmental sample | 28.3 | 8.98 | 31.5 |
| 15 | | 211 | JzSedJan11_10004914 | Environmental sample | 27.5 | 8.98 | 30.0 |
| 16 | | 212 | JGI20127J14776_100005829 | Environmental sample | 31.7 | 9.03 | 34.0 |
| 17 | | 213 | JzSedJan11_10131031 | Environmental sample | 30.9 | 6.73 | 31.5 |
| 18 | | 214 | YNPsite06_CeleraDRAFT_160970 | Environmental sample | 28.0 | 6.22 | 26.5 |
| 19 | | 215 | GxsBSedJan11_10061611 | Environmental sample | 28.4 | 5.59 | 34.0 |
| 20 | 3 | 301 | YNPsite06_CeleraDRAFT_367810 | Environmental sample | 54.1 | 5.86 | 22.5 |
| 21 | | 302 | YNPsite16_CeleraDRAFT_71360 | Environmental sample | 30.7 | 7.06 | 23.5 |
| 22 | | 303 | YNPsite16_CeleraDRAFT_248770 | Environmental sample | 54.4 | 6.00 | 37.0 |
| 23 | | 304 | YNP11_222720 | Environmental sample | 38.9 | 9.1 | 26.0 |
| 24 | | 305 | GxsBSedJan11_10251181 | Environmental sample | 27.8 | 6.5 | 25.5 |
| 25 | | 306 | GxsBSedJan11_10009658 | Environmental sample | 27.2 | 6.01 | 32.1 |
| 26 | | 307 | JGI20132J14458_10325381 | Environmental sample | 30.7 | 9.66 | 21.1 |
| 27 | | 308 | JzSedJan11_10355852 | Environmental sample | 27.7 | 8.35 | 33.0 |
| 28 | 4 | 401 | ACC95208.1 | uncultured bacterium | 360.0 | 5.40 | 30.0 |
| 29 | | 402 | WP_101893885.1 | Ketobacter alkanivorans | 360.7 | 5.57 | 32.0 |
| 30 | | 403 | RLU00646.1 | Ketobacter sp. | 353.9 | 4.52 | 31.0 |
| 31 | | 404 | WP_012854926.1 | Thermomonospora | 329.5 | 5.83 | 29.0 |
| | | | | curvata | | | |
| 32 | | 405 | WP_082414832.1 | Actinobacteria | 318.5 | 4.37 | 29.0 |
| | | | | bacterium | | | |
| 33 | | 406 | ODU60407.1 | Comamonadaceae | 298.2 | 8.30 | 31.5 |
| | | | | bacterium | | | |
| 34 | | 407 | WP_117215036.1 | Micromonosporaceae | 247.8 | 7.68 | 41.5 |
| | | | | bacterium | | | |
| 35 | | 408 | RCL73670.1 | Flavobacteriales | 137.9 | 4.29 | 40.0 |
| | | | | bacterium | | | |
| 36 | | 409 | RLT92980.1 | Ketobacter sp. | 122.8 | 7.75 | 29.0 |
| 37 | | 410 | RLT88027.1 | Alcanivoracaceae | 111.0 | 6.4 | 30.2 |
| | | | | bacterium | | | |
| 38 | | 411 | RLU03930.1 | Ketobacter sp. | 104.9 | 4.75 | 29.5 |
| 39 | | 412 | WP_101893509.1 | Ketobacter alkanivorans | 114.5 | 8.49 | 30.2 |
| 40 | | 413 | WP_115481747.1 | Robinsoniella sp. | 104.2 | 9.43 | 34.0 |
| 41 | 5 | 501 | 4EB0_A | uncultured bacterium | 355.1 | 9.32 | 28.0 |
| 42 | | 502 | PKO68961.1 | Betaproteobacteria | 335.5 | 9.49 | 28.0 |
| | | | | bacterium | | | |
| 43 | | 503 | EGD44994.1 | Nocardioidaceae | 296.7 | 5.10 | 28.0 |
| | | | | bacterium | | | |
| 44 | | 504 | WP_062195544.1 | Caldimonas | 314.9 | 9.26 | 29.5 |
| | | | | taiwanensis + D57 | | | |
| 45 | | 505 | OGP67040.1 | Deltaproteobacteria | 228.9 | 9.26 | 27.5 |
| | | | | bacterium | | | |
| 46 | 6 | 601 | WP_012851645.1 | Thermomonospora | 383.2 | 8.93 | 29.0 |
| | | | | curvata | | | |
| 47 | | 602 | WP_012850775.1 | Thermomonospora | 377.4 | 6.08 | 29.0 |
| | | | | curvata | | | |
| 48 | | 603 | WP_119925005.1 | Streptosporangiaceae | 377.7 | 5.82 | 28.5 |
| | | | | bacterium | | | |
| 49 | | 604 | WP_113973098.1 | Micromonospora sp. | 364.5 | 6.08 | 27.5 |
| 50 | | 605 | WP_106963453.1 | Actinomycetia | 369.4 | 6.42 | 29.0 |
| 51 | | 606 | WP_078759821.1 | Marinactinospora | 365.7 | 4.43 | 29.0 |
| | | | | thermotolerans | | | |
| 52 | | 607 | WP_107095481.1 | Actinobacteria | 378.2 | 5.47 | 28.0 |
| | | | | bacterium | | | |
| 53 | | 608 | WP_119951510.1 | Frankiales bacterium | 355.0 | 6.30 | 28.0 |
| 54 | | 609 | WP_125778035.1 | Promicromonosporaceae | 369.3 | 5.39 | 28.5 |
| | | | | bacterium | | | |
| 55 | | 610 | WP_125089638.1 | Saccharopolyspora sp. | 347.8 | 4.48 | 29.0 |
| 56 | | 611 | WP_093412886.1 | Saccharopolyspora flava | 353.5 | 4.31 | 28.5 |
| 57 | | 612 | OWY58880.1 | cyanobacterium TDX16 | 214.0 | 6.4 | 19.0 |
| 58 | 7 | 701 | WP_104613137.1 | Thermobifida fusca | 435.8 | 8.52 | 29.0 |
| 59 | | 702 | ADM47605.1 | Thermobifida fusca | 433.5 | 6.3 | 29.0 |
| 60 | | 703 | ADV92528.1 | Thermobifida fusca | 432.0 | 7.02 | 28.5 |
| 61 | | 704 | CBY05529.1 | Thermobifida fusca | 430.5 | 8.50 | 29.0 |
| 62 | | 705 | AAZ54920.1 | Thermobifida fusca | 426.2 | 6.97 | 29.0 |
| 63 | | 706 | CAH17554.1 | Thermobifida fusca | 425.6 | 8.5 | 29.0 |
| 64 | | 707 | ADV92525.1 | Thermobifida alba | 424.8 | 6.59 | 28.5 |
| 65 | | 708 | BAI99230.2 | Thermobifida alba | 414.4 | 5.74 | 29.0 |
| 66 | | 709 | WP_068752972.1 | Thermobifida | 411.8 | 6.30 | 29.0 |
| | | | | cellulosilytica | | | |
| 67 | | 710 | AFA45122.1 | Thermobifida | 405.8 | 5.24 | 29.0 |
| | | | | halotolerans | | | |
| 68 | | 711 | WP_083947829.1 | Thermobifida | 403.9 | 5.87 | 29.0 |
| | | | | cellulosilytica | | | |
| 69 | | 712 | RII04304.1 | Thermobifida | 182.2 | 4.47 | 13.0 |
| | | | | halotolerans | | | |
| 70 | | 713 | RII04310.1 | Thermobifida | 180.9 | 4.67 | 13.5 |
| | | | | halotolerans | | | |
| 71 | | 714 | CDN67547.1 | Thermobifida fusca | 437.5 | 6.59 | 29.0 |
| 72 | | 715 | ALF04778.1 | Thermobifida fusca | 437.2 | 6.30 | 28.5 |
| 73 | | 716 | 5LUK_A | Thermobifida | 426.6 | 6.21 | 29.0 |
| | | | | cellulosilytica | | | |
| 74 | | 717 | 3VIS_A | Thermobifida alba | 408.1 | 5.96 | 29.0 |

TABLE 5
Enzymes and reaction conditions tested in 168 h time course experiments.
Selectivity ratio provides the mass ratio of products at 168
h and preference for amorphous PET film (A) or crystalline PET
powder (C) is noted. Reaction conditions tested that are not
shown in FIG. 2B are noted with an asterisk (*).

| | | Reaction Condition | Selectivity Ratio at 168 h | |
|---|---|---|---|---|
| | Enzyme ID | (pH/Temperature) | (mass ratio) | |
| 1 | BTA-1 | H7.5/60° C. | 8.05 | (A) |
| 2 | LCC_WT | NP7.5/60° C. | 3.67 | (A) |
| 3 | LCC ICCG | NP7.5/70° C. | 4.56 | (A) |
| 4 | LCC ICCG | C6/60° C. (*) | 5.08 | (A) |
| 5 | 102 | C6/60° C. | 7.84 | (C) |
| 6 | 202 | NP7.5/70° C. | 1.46 | (C) |
| 7 | 211 | NP7.5/70° C. | 1.24 | (A) |
| 8 | 407 | G9/50° C. | 1.23 | (C) |
| 9 | 504 | B8/50° C. | 5.64 | (C) |
| 10 | 601 | NP7.5/60° C. | 1.86 | (C) |
| 11 | 606 | G9/60° C. | 3.30 | (C) |
| 12 | 606 | NP7.5/60° C. (*) | 3.33 | (C) |
| 13 | 611 | C6/50° C. | 1.24 | (C) |
| 14 | 611 | NP7.5/50° C. (*) | 10.31 | (C) |
| 15 | 701 | NP7.5/60° C. | 4.73 | (A) |
| 16 | 704 | NP7/60° C. | 7.41 | (A) |
| 17 | 704 | NP7.5/60° C. (*) | 10.46 | (A) |
| 18 | 714 | NP7/60° C. | 1.95 | (A) |
| 19 | 716 | NP7.5/60° C. | 3.08 | (A) |

TABLE 6
Tm data for selected proteins.

| | Mean | Tₘ | |
|---|---|---|---|
| Enzyme | Tₘ | s.d. | |
| ID | (° C.) | (° C.) | Buffer |
| 102 | 65.96 | ±0.28 | NP7.5 |
| 202 | 75.13 | ±0.06 | NP7.5 |
| 306 | 92.57 | ±0.02 | NP7.5 |
| 407 | 68.20 | ±0.04 | NP7.5 |
| 501 | 86.91 | ±0.12 | NP7.5 |
| 504 | 67.25 | ±0.03 | NP7.5 |
| 601 | 67.18 | ±0.04 | NP7.5 |
| 606 | 53.90 | ±0.11 | NP7.5 + 0.3M |
| | | | NaCl |
| 611 | 76.21 | ±0.05 | NP7.5 |
| 701 | 70.28 | ±0.03 | NP7.5 |
| 702 | 65.57 | ±0.03 | NP7.5 |
| 703 | 70.86 | ±0.09 | NP7.5 |
| 704 | 69.93 | ±0.08 | NP7.5 |
| 705 | 69.02 | ±0.05 | NP7.5 |
| 706 | 68.35 | ±0.10 | NP7.5 |
| 709 | 56.05 | ±0.05 | NP7.5 |
| 711 | 54.16 | ±0.03 | NP7.5 |
| 714 | 69.96 | ±0.08 | NP7.5 |
| 715 | 71.83 | ±0.03 | NP7.5 |
| 716 | 67.71 | ±0.15 | NP7.5 |
| BTA-1 | 71.94 | ±0.03 | NP7.5 |

Disclosed herein are predicted and verified PET hydrolase enzymes, their activity, and their nucleic acid and amino acid sequences. In an embodiment, as disclosed in Appendix A, are amino acid sequences of PET hydrolase enzymes that have been identified. In an embodiment, the amino acid sequences disclosed in Appendix A each begin with a methionine. In an embodiment, some of the identified sequences have been cloned, and the enzymes that they encode for have been expressed, purified and their PET hydrolase activity has been determined. In an embodiment, the PET hydrolase enzymes disclosed herein possess desirable traits that are leveraged in the design and engineering of enzyme formulations targeted to degrade specific polymers. In an embodiment, the PET enzymes disclosed herein have measurable PET degrading activity and, may be active for degrading polyester polyurethanes.

In an embodiment, computational methods and other algorithms are used to predict and identify nucleic acid and amino acid sequences for active PET hydrolase enzymes. In an embodiment, the use of algorithms is contemplated to predict secondary, tertiary and quaternary structures for the predicted PET hydrolase enzymes.

Disclosed herein are seven clade groups of PET hydrolase enzymes that were identified using the methods disclosed herein and the accession numbers of the putative and actual PET hydrolase enzyme members of the clades are disclosed in Table 7.

TABLE 7

| PETcan | | | Max | |
|---|---|---|---|---|
| group | Seq ID Code | Accession | shared ID | ID shared with |
| Group1 | PETcan_101 | Ga0073930_10154211 | 38.21 | Ga0116197_16468841 |
| | PETcan_102 | Ga0073929_100051119 | 100.00 | Ga0073929_100051119 |
| | PETcan_103 | Ga0116197_16468841 | 45.05 | shallow_100244311 |
| | PETcan_104 | JGI24721J44947_100139617 | 23.50 | Ga0116197_16468841 |
| Group 2 | PETcan_201 | shallow_100028175 | 100.00 | shallow_100028175 |
| | PETcan_202 | Ga0073932_10599092 | 99.71 | Ga0073934_113259931 |
| | PETcan_203 | Ga0123519_100040842 | 22.84 | Deep_10535451 |
| | PETcan_204 | Ga0116196_10092351 | 100.00 | Deep_10535451 |
| | PETcan_205 | Ga0129302_15272001 | 74.87 | Ga0073933_11240711 |
| | PETcan_206 | Ga0167616_10026342 | 95.44 | Ga0116196_10092351 |
| | PETcan_207 | shallow_10026563 | 100.00 | Ga0073933_11240711 |
| | PETcan_208 | Ga0116211_10708811 | 41.31 | Deep_10535451 |
| | PETcan_209 | Ga0073934_113259931 | 99.71 | Ga0073932_10599092 |
| | PETcan_210 | Ga0073934_112999861 | 90.87 | Ga0073930_10827831 |
| | PETcan_211 | Ga0073930_10827831 | 90.87 | Ga0073934_112999861 |
| | PETcan_212 | Ga0073934_109541201 | 25.55 | shallow_100028175 |
| | PETcan_213 | Ga0116197_12958211 | 71.86 | Ga0073930_10827831 |
| | PETcan_214 | Ga0073934_100093435 | 95.82 | Ga0073932_10599092 |
| | PETcan_215 | Ga0129302_11414112 | 37.69 | Ga0073932_10599092 |
| Group 3 | PETcan_301 | Ga0073934_104567521 | 100.00 | Ga0073930_100020586 |
| | PETcan_302 | Ga0073934_107020181 | 37.16 | Ga0073934_104567521 |
| | PETcan_303 | Ga0073934_107895621 | 31.17 | Ga0116211_13093651 |
| | PETcan_304 | Ga0073933_100024419 | 99.42 | shallow_100088918 |
| | PETcan_305 | Ga0116211_13093651 | 31.17 | Ga0073934_107895621 |
| | PETcan_306 | Ga0129302_11993521 | 30.51 | Ga0167616_10021342 |
| | PETcan_307 | Ga0167616_10021342 | 30.51 | Ga0129302_11993521 |
| | PETcan_308 | Ga0116197_10916912 | 22.61 | Ga0129302_11993521 |
| Group 4 | PETcan_401 | ACC95208.1 | 61.69 | RLU00646.1 |
| | PETcan_402 | WP_101893885.1 | 77.88 | RLU00646.1 |
| | PETcan_403 | RLU00646.1 | 77.88 | WP_101893885.1 |
| | PETcan_404 | WP_012854926.1 | 62.71 | WP_082414832.1 |
| | PETcan_405 | WP_082414832.1 | 62.71 | WP_012854926.1 |
| | PETcan_406 | ODU60407.1 | 48.85 | RLU00646.1 |
| | PETcan_407 | WP_117215036.1 | 49.01 | WP_082414832.1 |
| | PETcan_408 | RCL73670.1 | 31.82 | ACC95208.1 |
| | PETcan_409 | RLT92980.1 | 85.13 | WP_101893509.1 |
| | PETcan_410 | RLT88027.1 | 83.39 | WP_101893509.1 |
| | PETcan_411 | RLU03930.1 | 69.52 | RLT92980.1 |
| | PETcan_412 | WP_101893509.1 | 85.13 | RLT92980.1 |
| | PETcan_413 | WP_115481747.1 | 62.08 | RLT92980.1 |
| Group 5 | PETcan_501 | pdb\|4EB0\|A | 100.00 | pdb\|4EB0\|A |
| | PETcan_502 | PKO68961.1 | 53.10 | pdb\|4EB0\|A |
| | PETcan_503 | EGD44994.1 | 53.10 | pdb\|4EB0\|A |
| | PETcan_504 | WP_062195544.1 | 51.94 | pdb\|4EB0\|A |
| | PETcan_505 | OGP67040.1 | 47.52 | PKO68961.1 |
| Group 6 | PETcan_601 | WP_012851645.1 | 78.89 | WP_012850775.1 |
| | PETcan_602 | WP_012850775.1 | 78.89 | WP_012851645.1 |
| | PETcan_603 | WP_119925005.1 | 71.08 | WP_106963453.1 |
| | PETcan_604 | WP_113973098.1 | 70.21 | WP_012850775.1 |
| | PETcan_605 | WP_106963453.1 | 81.18 | KPI31299.1 |
| | PETcan_606 | WP_078759821.1 | 62.95 | WP_119925005.1 |
| | PETcan_607 | WP_107095481.1 | 100.00 | KPI31299.1 |
| | PETcan_608 | WP_119951510.1 | 66.89 | WP_119925005.1 |
| | PETcan_609 | WP_125778035.1 | 73.87 | WP_106963453.1 |
| | PETcan_610 | WP_125089638.1 | 84.30 | WP_093412886.1 |
| | PETcan_611 | WP_093412886.1 | 84.30 | WP_125089638.1 |
| | PETcan_612 | OWY58880.1 | 62.29 | KPI31299.1 |
| Group 7 | PETcan_701 | WP_104613137.1 | 99.24 | ADV92528.1 |
| | PETcan_702 | ADM47605.1 | 98.85 | WP_011291330.1 |
| | PETcan_703 | ADV92528.1 | 99.24 | WP_104613137.1 |
| | PETcan_704 | CBY05529.1 | 97.67 | CAH17554.1 |
| | PETcan_705 | AAZ54920.1 | 99.62 | ADV92527.1 |
| | PETcan_706 | CAH17554.1 | 99.00 | AAZ54920.1 |
| | PETcan_707 | ADV92525.1 | 98.47 | ADV92526.1 |
| | PETcan_708 | BAI99230.2 | 93.92 | BAK48590.1 |
| | PETcan_709 | WP_068752972.1 | 90.08 | ADV92527.1 |
| | PETcan_710 | AFA45122.1 | 77.86 | BAK48590.1 |
| | PETcan_711 | WP_083947829.1 | 82.75 | WP_068752972.1 |
| | PETcan_712 | RII04304.1 | 83.95 | RII04310.1 |
| | PETcan_713 | RII04310.1 | 83.95 | RII04304.1 |
| | PETcan_714 | CDN67547.1 | 100.00 | PPS86343.1 |
| | PETcan_715 | ALF04778.1 | 99.62 | ADV92526.1 |
| | PETcan_716 | pdb\|5LUK\|A | 99.24 | ADV92527.1 |
| | PETcan_717 | pdb\|3VIS\|A | 100.00 | BAK48590.1 |

Table 8 discloses PETcan group clades and controls, their respective sequence identifiers used herein, their respective PET hydrolase activity levels, their respective amino acid sequences, their respective nucleotide sequences, the expression conditions of the studied enzymes as well as additional information regarding yield of the expressed PET hydrolases.

TABLE 8

| | | | | Nucleotide Sequence | |
|---|---|---|---|---|---|
| | | | | (excludes flanking | |
| | | | | restriction sites: | Expres- |
| PET | Seq | | | 5′-CATATG and | sion |
| can | ID | Activity | | CTCGAG-3′ and C- | Condi- |
| group | # | Level | Protein Sequence | terminal His tag) | tions |
| Con- | LCCWT | 3 | MSNPYQRGPNPTRSALTADGPFS | TCTAACCCGTACCAGCGCGGAC | 20° |
| trols | | | VATYTVSRLSVSGFGGGVIYYPT | CGAACCCGACCCGTTCTGCGTT | C./20 |
| | | | GTSLTFGGIAMSPGYTADASSLA | AACCGCTGATGGTCCGTTTTCC | hIP |
| | | | WLGRRLASHGFVVLVINTNSRFD | GTGGCTACCTACACCGTTTCTC | TG2xYT |
| | | | YPDSRASQLSAALNYLRTSSPSA | GTCTGTCCGTTTCCGGTTTTGGT | |
| | | | VRARLDANRLAVAGHSMGGGG | GGTGGTGTTATCTACTATCCGA | |
| | | | TLRIAEQNPSLKAAVPLTPWHTD | CTGGTACCTCTCTGACCTTCGG | |
| | | | KTFNTSVPVLIVGAEADTVAPVS | CGGTATCGCGATGTCCCCGGGT | |
| | | | QHAIPFYQNLPSTTPKVYVELDN | TACACCGCTGATGCTTCCTCTCT | |
| | | | ASHFAPNSNNAAISVYTISWMKL | GGCGTGGCTGGGTCGTCGCCTG | |
| | | | WVDNDTRYRQFLCNVNDPALSD | GCGAGCCACGGTTTTGTTGTTC | |
| | | | FRTNNRHCQLEHHHHHH | TGGTTATCAACACGAACTCTCG | |
| | | | | TTTCGACTATCCGGACTCCCGT | |
| | | | | GCCTCGCAACTGTCTGCTGCGC | |
| | | | | TGAACTACCTGCGTACGTCGTC | |
| | | | | ACCTTCAGCGGTCCGTGCACGC | |
| | | | | CTGGATGCCAATCGTCTGGCTG | |
| | | | | TGGCGGGTCACAGCATGGGCGG | |
| | | | | TGGCGGTACCCTGCGTATTGCT | |
| | | | | GAACAGAACCCGTCCCTGAAAG | |
| | | | | CTGCAGTGCCACTGACTCCGTG | |
| | | | | GCATACCGACAAAACGTTCAAC | |
| | | | | ACCAGTGTTCCGGTACTGATCG | |
| | | | | TAGGCGCAGAAGCGGACACCG | |
| | | | | TAGCACCGGTTTCCCAGCACGC | |
| | | | | AATCCCGTTCTACCAGAACCTG | |
| | | | | CCGAGCACCACTCCAAAAGTAT | |
| | | | | ACGTTGAACTGGACAACGCCTC | |
| | | | | GCACTTCGCTCCGAACTCGAAC | |
| | | | | AACGCTGCGATTAGCGTGTACA | |
| | | | | CCATCTCCTGGATGAAACTGTG | |
| | | | | GGTTGATAACGATACCCGTTAT | |
| | | | | CGCCAATTCCTGTGTAACGTGA | |
| | | | | ACGATCCGGCTCTCTCAGATTT | |
| | | | | TCGTACCAACAACCGTCATTGC | |
| | | | | CAA | |
| | LCCICCG | 3 | MSNPYQRGPNPTRSALTADGPFS | TCTAACCCGTACCAGCGCGGAC | |
| | | | VATYTVSRLSVSGFGGGVIYYPT | CGAACCCGACCCGTTCTGCGTT | |
| | | | GTSLTFGGIAMSPGYTADASSLA | AACCGCTGATGGTCCGTTTTCC | |
| | | | WLGRRLASHGFVVLVINTNSRFD | GTGGCTACCTACACCGTTTCTC | |
| | | | GPDSRASQLSAALNYLRTSSPSA | GTCTGTCCGTTTCCGGTTTTGGT | |
| | | | VRARLDANRLAVAGHSMGGGG | GGTGGTGTTATCTACTATCCGA | |
| | | | TLRIAEQNPSLKAAVPLTPWHTD | CTGGTACCTCTCTGACCTTCGG | |
| | | | KTFNTSVPVLIVGAEADTVAPVS | CGGTATCGCGATGTCCCCGGGT | |
| | | | QHAIPFYQNLPSTTPKVYVELCN | TACACCGCTGATGCTTCCTCTCT | |
| | | | ASHIAPNSNNAAISVYTISWMKL | GGCGTGGCTGGGTCGTCGCCTG | |
| | | | WVDNDTRYRQFLCNVNDPALCD | GCGAGCCACGGTTTTGTTGTTC | |
| | | | FRTNNRHCQLEHHHHHH | TGGTTATCAACACGAACTCTCG | |
| | | | | TTTCGACGGCCCGGACTCCCGT | |
| | | | | GCCTCGCAACTGTCTGCTGCGC | |
| | | | | TGAACTACCTGCGTACGTCGTC | |
| | | | | ACCTTCAGCGGTCCGTGCACGC | |
| | | | | CTGGATGCCAATCGTCTGGCTG | |
| | | | | TGGCGGGTCACAGCATGGGCGG | |
| | | | | TGGCGGTACCCTGCGTATTGCT | |
| | | | | GAACAGAACCCGTCCCTGAAAG | |
| | | | | CTGCAGTGCCACTGACTCCGTG | |
| | | | | GCATACCGACAAAACGTTCAAC | |
| | | | | ACCAGTGTTCCGGTACTGATCG | |
| | | | | TAGGCGCAGAAGCGGACACCG | |
| | | | | TAGCACCGGTTTCCCAGCACGC | |
| | | | | AATCCCGTTCTACCAGAACCTG | |
| | | | | CCGAGCACCACTCCAAAAGTAT | |
| | | | | ACGTTGAACTGTGCAACGCCTC | |
| | | | | GCACATTGCTCCGAACTCGAAC | |
| | | | | AACGCTGCGATTAGCGTGTACA | |
| | | | | CCATCTCCTGGATGAAACTGTG | |
| | | | | GGTTGATAACGATACCCGTTAT | |
| | | | | CGCCAATTCCTGTGTAACGTGA | |
| | | | | ACGATCCGGCTCTCTGCGATTT | |
| | | | | TCGTACCAACAACCGTCATTGC | |
| | | | | CAA | |
| | LCCWCCG | 3 | MSNPYQRGPNPTRSALTADGPFS | TCTAACCCGTACCAGCGCGGAC | |
| | | | VATYTVSRLSVSGFGGGVIYYPT | CGAACCCGACCCGTTCTGCGTT | |
| | | | GTSLTFGGIAMSPGYTADASSLA | AACCGCTGATGGTCCGTTTTCC | |
| | | | WLGRRLASHGFVVLVINTNSRFD | GTGGCTACCTACACCGTTTCTC | |
| | | | GPDSRASQLSAALNYLRTSSPSA | GTCTGTCCGTTTCCGGTTTTGGT | |
| | | | VRARLDANRLAVAGHSMGGGG | GGTGGTGTTATCTACTATCCGA | |
| | | | TLRIAEQNPSLKAAVPLTPWHTD | CTGGTACCTCTCTGACCTTCGG | |
| | | | KTFNTSVPVLIVGAEADTVAPVS | CGGTATCGCGATGTCCCCGGGT | |
| | | | QHAIPFYQNLPSTTPKVYVELCN | TACACCGCTGATGCTTCCTCTCT | |
| | | | ASHWAPNSNNAAISVYTISWMK | GGCGTGGCTGGGTCGTCGCCTG | |
| | | | LWVDNDTRYRQFLCNVNDPALC | GCGAGCCACGGTTTTGTTGTTC | |
| | | | DFRTNNRHCQLEHHHHHH | TGGTTATCAACACGAACTCTCG | |
| | | | | TTTCGACGGCCCGGACTCCCGT | |
| | | | | GCCTCGCAACTGTCTGCTGCGC | |
| | | | | TGAACTACCTGCGTACGTCGTC | |
| | | | | ACCTTCAGCGGTCCGTGCACGC | |
| | | | | CTGGATGCCAATCGTCTGGCTG | |
| | | | | TGGCGGGTCACAGCATGGGCGG | |
| | | | | TGGCGGTACCCTGCGTATTGCT | |
| | | | | GAACAGAACCCGTCCCTGAAAG | |
| | | | | CTGCAGTGCCACTGACTCCGTG | |
| | | | | GCATACCGACAAAACGTTCAAC | |
| | | | | ACCAGTGTTCCGGTACTGATCG | |
| | | | | TAGGCGCAGAAGCGGACACCG | |
| | | | | TAGCACCGGTTTCCCAGCACGC | |
| | | | | AATCCCGTTCTACCAGAACCTG | |
| | | | | CCGAGCACCACTCCAAAAGTAT | |
| | | | | ACGTTGAACTGTGCAACGCCTC | |
| | | | | GCACTGGGCTCCGAACTCGAAC | |
| | | | | AACGCTGCGATTAGCGTGTACA | |
| | | | | CCATCTCCTGGATGAAACTGTG | |
| | | | | GGTTGATAACGATACCCGTTAT | |
| | | | | CGCCAATTCCTGTGTAACGTGA | |
| | | | | ACGATCCGGCTCTCTGCGATTT | |
| | | | | TCGTACCAACAACCGTCATTGC | |
| | | | | CAA | |
| | Is.PET | 2 | MNFPRASRLMQAAVLGGLMAVS | aacttcccccgtgcctcgcgcct | 20° |
| | aseWT | | AAATAQTNPYARGPNPTAASLE | tatgcaggctgctgtgctgggcg | C./20 |
| | | | ASAGPFTVRSFTVSRPSGYGAGT | gccttatggccgtttccgcagcg | hIP |
| | | | VYYPTNAGGTVGAIAIVPGYTAR | gccaccgcgcagaccaatccgta | TG2xYT |
| | | | QSSIKWWGPRLASHGFVVITIDT | tgcgcgcggccccaaccctaccg | |
| | | | NSTLDQPSSRSSQQMAALRQVAS | ccgcctcgttggaagccagcgcg | |
| | | | LNGTSSSPIYGKVDTARMGVMG | ggaccctttaccgttcgtagctt | |
| | | | WSMGGGGSLISAANNPSLKAAA | taccgttagccgtccgtccggat | |
| | | | PQAPWDSSTNFSSVTVPTLIFACE | atggtgcagggaccgtctattac | |
| | | | NDSIAPVNSSALPIYDSMSRNAK | ccaaccaatgcaggcggcaccgt | |
| | | | QFLEINGGSHSCANSGNSNQALI | tggcgcgattgcaatcgtccccg | |
| | | | GKKGVAWMKRFMDNDTRYSTF | ggtacaccgcgcgtcaaagcagc | |
| | | | ACENPNSTRVSDFRTANCSLEHH | attaagtggtggggtccgcgctt | |
| | | | HHHH | agctagccatggctttgtggtta | |
| | | | | ttaccatcgatacgaacagcact | |
| | | | | ctagaccagcccagcagccgtag | |
| | | | | ctcgcaacagatggccgcgcttc | |
| | | | | gtcaagttgcgagcttgaacggg | |
| | | | | accagcagtagcccgatttacgg | |
| | | | | aaaggtcgatactgcccgcatgg | |
| | | | | gtgtgatgggctggtcaatgggg | |
| | | | | ggcggcggttcacttattagcgc | |
| | | | | cgcgaacaacccgagtttaaaag | |
| | | | | cagcggcaccgcaggcgccatgg | |
| | | | | gactcttcaaccaacttcagcag | |
| | | | | tgttaccgtgccgacgctgattt | |
| | | | | tcgcgtgcgagaatgatagcatt | |
| | | | | gcaccggtgaacagcagcgcgct | |
| | | | | gccgatttatgatagcatgtccc | |
| | | | | gcaacgcaaaacagtttctggaa | |
| | | | | attaacggcggtagccactcttg | |
| | | | | tgccaactctgggaacagcaacc | |
| | | | | aggcactgatcggaaaaaaaggg | |
| | | | | gttgcatggatgaaacgattcat | |
| | | | | ggataatgacacccgttactcaa | |
| | | | | ccttcgcctgtgagaatcccaac | |
| | | | | agcacacgcgtgtcggattttcg | |
| | | | | caccgcgaactgttcc | |
| | Is.PET | 2 | MNFPRASRLMQAAVLGGLMAVS | aacttcccccgtgcctcgcgcct | 20° |
| | asedm | | AAATAQTNPYARGPNPTAASLE | tatgcaggctgctgtgctgggcg | C./20 |
| | | | ASAGPFTVRSFTVSRPSGYGAGT | gccttatggccgtttccgcagcg | hIP |
| | | | VYYPTNAGGTVGAIAIVPGYTAR | gccaccgcgcagaccaatccgta | TG2xYT |
| | | | QSSIKWWGPRLASHGFVVITIDT | tgcgcgcggccccaaccctaccg | |
| | | | NSTLDQPSSRSSQQMAALRQVAS | ccgcctcgttggaagccagcgcg | |
| | | | LNGTSSSPIYGKVDTARMGVMG | ggaccctttaccgttcgtagctt | |
| | | | HSMGGGGSLISAANNPSLKAAAP | taccgttagccgtccgtccggat | |
| | | | QAPWDSSTNFSSVTVPTLIFACEN | atggtgcagggaccgtctattac | |
| | | | DSIAPVNSSALPIYDSMSRNAKQF | ccaaccaatgcaggcggcaccgt | |
| | | | LEINGGSHFCANSGNSNQALIGK | tggcgcgattgcaatcgtccccg | |
| | | | KGVAWMKRFMDNDTRYSTFAC | ggtacaccgcgcgtcaaagcagc | |
| | | | ENPNSTRVSDFRTANCSLEHHHH | attaagtggtggggtccgcgctt | |
| | | | HH | agctagccatggctttgtggtta | |
| | | | | ttaccatcgatacgaacagcact | |
| | | | | ctagaccagcccagcagccgtag | |
| | | | | ctcgcaacagatggccgcgcttc | |
| | | | | gtcaagttgcgagcttgaacggg | |
| | | | | accagcagtagcccgatttacgg | |
| | | | | aaaggtcgatactgcccgcatgg | |
| | | | | gtgtgatgggccactcaatgggg | |
| | | | | ggcggcggttcacttattagcgc | |
| | | | | cgcgaacaacccgagtttaaaag | |
| | | | | cagcggcaccgcaggcgccatgg | |
| | | | | gactcttcaaccaacttcagcag | |
| | | | | tgttaccgtgccgacgctgattt | |
| | | | | tcgcgtgcgagaatgatagcatt | |
| | | | | gcaccggtgaacagcagcgcgct | |
| | | | | gccgatttatgatagcatgtccc | |
| | | | | gcaacgcaaaacagtttctggaa | |
| | | | | attaacggcggtagccacttctg | |
| | | | | tgccaactctgggaacagcaacc | |
| | | | | aggcactgatcggaaaaaaaggg | |
| | | | | gttgcatggatgaaacgattcat | |
| | | | | ggataatgacacccgttactcaa | |
| | | | | ccttcgcctgtgagaatcccaac | |
| | | | | agcacacgcgtgtcggattttcg | |
| | | | | caccgcgaactgttcc | |
| | TfCut | 2 | MANPYERGPNPTDALLEASSGPF | gctaacccgtatgaacgcggccc | 20° |
| | | | SVSEENVSRLSASGFGGGTIYYPR | gaaccctacggacgccctgctgg | C./20 |
| | | | ENNTYGAVAISPGYTGTEASIAW | aagcatcctctggtccgttctca | hIP |
| | | | LGERIASHGFVVITIDTITTLDQPD | gtgtccgaagaaaacgtgtcccg | TG2xYT |
| | | | SRAEQLNAALNHMINRASSTVRS | tcttagcgcttctggtttcggtg | |
| | | | RIDSSRLAVMGHSMGGGGTLRL | gcggcactatctactacccgcgt | |
| | | | ASQRPDLKAAIPLTPWHLNKNW | gagaacaacacttatggtgctgt | |
| | | | SSVTVPTLIIGADLDTIAPVATHA | ggctattagcccgggctacactg | |
| | | | KPFYNSLPSSISKAYLELDGATHF | gcactgaagcgtccattgcgtgg | |
| | | | APNIPNKIIGKYSVAWLKRFVDN | ctgggtgaacgcatcgcttccca | |
| | | | DTRYTQFLCPGPRDGLFGEVEEY | tggattcgttgttattaccattg | |
| | | | RSTCPFLEHHHHHH | acaccatcacgaccctcgaccag | |
| | | | | ccggactcccgcgctgaacagct | |
| | | | | gaacgcggctctcaaccatatga | |
| | | | | tcaaccgtgcttcttccaccgtc | |
| | | | | cgttctcgcatcgacagctctcg | |
| | | | | cctggctgttatgggtcacagca | |
| | | | | tgggtggcggtggtaccctgcgc | |
| | | | | ctggcatcccagcgcccggacct | |
| | | | | gaaagctgctatcccgctcactc | |
| | | | | cgtggcatctgaacaaaaactgg | |
| | | | | tcttctgttaccgtcccgaccct | |
| | | | | gatcatcggcgccgatctggata | |
| | | | | ccattgctccggttgcgactcat | |
| | | | | gctaaaccgttctacaacagcct | |
| | | | | tccgtcttctatctccaaggctt | |
| | | | | acctggaactggatggagcaact | |
| | | | | cacttcgccccgaacattccgaa | |
| | | | | taaaatcatcggcaaatattccg | |
| | | | | ttgcttggctgaaacgtttcgta | |
| | | | | gacaatgatacccgttatactca | |
| | | | | gttcctgtgcccgggcccgcgcg | |
| | | | | acggcctgtttggtgaagttgag | |
| | | | | gagtatcgttccacctgcccgtt | |
| | | | | c | |
| Group2 | 202 | 1 | MVDITGNGMAATAPTDERIVDK | GTTGATATCACTGGCAACGGTA | 20° |
| | | | PLPQPQIRSGNVRAMPAARKLAQ | TGGCTGCTACCGCGCCGACCGA | C./20 |
| | | | EHGIDLSTLTGSGPGG | CGAACGTATTGTAGACAAACCT | hIP |
| | | | VIVKEDVERAITARAVPVSPLQR | CTGCCTCAGCCGCAGATTCGTT | TG2xYT |
| | | | VNFYSAGYRLDGLLYTPRHLPAG | CTGGTAACGTTCGTGCAATGCC | |
| | | | ERRPGVVLLVGYTY | GGCGGCTCGCAAGCTGGCGCAG | |
| | | | LKTMVMPDIAKVLNAAGYVALV | GAGCACGGTATTGACCTGTCCA | |
| | | | FDYRGFGESEGPRGRLIPLEQVA | CTCTGACCGGTAGCGGTCCAGG | |
| | | | DARAALTFLAEQSMV | TGGTGTTATCGTTAAAGAGGAC | |
| | | | DPDRLAVIGISLGGAHAITTAALD | GTCGAACGTGCAATCACCGCTC | |
| | | | QRVRAVVALEPPGHGARWLRSL | GTGCTGTTCCTGTATCTCCGCTG | |
| | | | RRHWEWRQFLSRLA | CAGCGTGTCAACTTTTATTCTGC | |
| | | | EDRRQRVLSGGSTMVDPLEIVLP | CGGTTATCGTCTGGACGGTCTG | |
| | | | DPESQAFLDQVAAEFPQMKVTLP | CTGTACACCCCGCGTCACCTGC | |
| | | | LESAEALIEYVSED | CAGCTGGTGAACGTAGACCGGG | |
| | | | LAGRIAPRPLLIIHSDADQLVPVA | TGTCGTTCTCCTGGTGGGTTAC | |
| | | | EAQAIAERAGSSAQLEIIPGMSHF | ACTTACCTCAAAACTATGGTAA | |
| | | | NWVMPGSPGFTR | TGCCGGACATCGCGAAAGTTCT | |
| | | | VTDSIVKFLRNTLPVSADN | GAACGCTGCGGGTTACGTTGCC | |
| | | | LEHHHHHH | CTGGTTTTCGACTACCGCGGCT | |
| | | | | TCGGCGAATCCGAGGGCCCGCG | |
| | | | | CGGTCGTCTAATCCCGTTAGAA | |
| | | | | CAAGTAGCTGATGCACGTGCAG | |
| | | | | CGCTGACCTTTCTGGCGGAACA | |
| | | | | GTCAATGGTTGATCCGGATCGT | |
| | | | | CTCGCGGTAATTGGCATTTCTCT | |
| | | | | GGGTGGTGCACATGCAATTACC | |
| | | | | ACTGCTGCACTGGATCAGCGTG | |
| | | | | TCCGCGCGGTCGTGGCTCTGGA | |
| | | | | ACCGCCAGGCCATGGTGCGCGT | |
| | | | | TGGCTGCGTAGCCTGCGTCGTC | |
| | | | | ACTGGGAATGGCGTCAGTTCCT | |
| | | | | GTCTCGTCTGGCTGAAGATCGT | |
| | | | | CGTCAGCGCGTGCTAAGCGGTG | |
| | | | | GCAGCACCATGGTTGACCCGCT | |
| | | | | GGAGATCGTTCTGCCAGACCCG | |
| | | | | GAGTCTCAGGCTTTCCTGGACC | |
| | | | | AAGTTGCCGCAGAATTTCCGCA | |
| | | | | GATGAAAGTGACGCTGCCGCTG | |
| | | | | GAATCTGCCGAAGCACTGATCG | |
| | | | | AATATGTGTCCGAAGACCTCGC | |
| | | | | CGGCCGTATCGCTCCGCGTCCA | |
| | | | | CTGCTGATCATTCACTCTGACG | |
| | | | | CCGACCAGCTGGTTCCGGTTGC | |
| | | | | GGAAGCTCAGGCGATCGCAGA | |
| | | | | GCGCGCGGGCTCTTCTGCACAG | |
| | | | | CTGGAGATCATTCCAGGCATGT | |
| | | | | CCCATTTCAATTGGGTAATGCC | |
| | | | | AGGCAGCCCGGGCTTCACTCGT | |
| | | | | GTTACTGATTCTATCGTTAAATT | |
| | | | | CCTGCGTAACACCCTGCCGGTA | |
| | | | | TCTGCGGACAAT | |
| | 204 | 1 | MVPSAGVGLSGVLHLPAGVSRP | GTGCCAAGCGCGGGTGTAGGTC | Auto |
| | | | VLFLHGFTGNKTESGRLYTDMA | TTTCTGGCGTCCTCCATCTGCCG | 28° |
| | | | RVLCSAGYAALREDFRG | GCTGGCGTTTCCCGCCCGGTGC | C./24 |
| | | | HGDSPLPFEEFRISLAVEDARNAA | TGTTCCTGCATGGTTTCACGGG | h |
| | | | GFLKNVPEVDGTRFGVVGLSMG | CAACAAGACGGAAAGCGGTCG | |
| | | | GGVAVSLAAGREDV | TTTGTACACCGACATGGCGCGC | |
| | | | GALVLLSPALDWPELFQRARGFF | GTTCTGTGTTCTGCGGGCTACG | |
| | | | RAEEGYVYWGPHRMRDVYAME | CAGCCTTGCGTTTCGATTTTCGT | |
| | | | TMNFSVMGLAEEIQAP | GGTCACGGTGATAGCCCTCTGC | |
| | | | TLIIHSVDDMVVPISQAKRFYEKL | CATTCGAGGAATTTCGTATCAG | |
| | | | KVEKKFIEIEHGGHVFDDYNVRR | TCTGGCAGTTGAAGACGCCCGT | |
| | | | RIEQEVLDWVKRH | AACGCGGCCGGTTTCCTGAAAA | |
| | | | LLEHHHHHH | ACGTACCGGAAGTGGACGGAA | |
| | | | | CTCGCTTTGGTGTAGTGGGCTT | |
| | | | | GTCTATGGGTGGCGGCGTGGCA | |
| | | | | GTGAGCCTGGCGGCTGGTCGCG | |
| | | | | AAGACGTTGGTGCGCTCGTGCT | |
| | | | | GCTTTCTCCGGCTCTGGATTGG | |
| | | | | CCTGAACTCTTCCAGCGTGCGC | |
| | | | | GTGGCTTCTTTCGTGCGGAAGA | |
| | | | | GGGCTACGTGTACTGGGGCCCG | |
| | | | | CACCGTATGCGCGATGTTTACG | |
| | | | | CTATGGAAACCATGAACTTCTC | |
| | | | | TGTAATGGGCCTGGCCGAAGAA | |
| | | | | ATCCAAGCGCCGACTCTGATCA | |
| | | | | TCCACTCTGTTGATGACATGGT | |
| | | | | TGTTCCGATTAGTCAAGCCAAA | |
| | | | | CGCTTCTATGAAAAACTGAAAG | |
| | | | | TAGAAAAAAAGTTTATCGAGAT | |
| | | | | CGAACACGGTGGTCACGTTTTT | |
| | | | | GATGACTACAACGTGCGTCGCC | |
| | | | | GTATCGAGCAGGAGGTTCTCGA | |
| | | | | CTGGGTGAAACGCCACCTG | |
| | 206 | 0 | MVPSAGVGLSGVLHLPAGVSRP | GTTCCATCCGCGGGTGTAGGCC | Auto + |
| | | | VLFLHGFTGNKTESGRLYTDMA | TGTCTGGCGTTCTTCACCTGCCG | NaCl |
| | | | RVLCSAGYAALREDFRC | GCAGGCGTAAGCCGCCCGGTGC | 25° |
| | | | HGDSPLPFEEFRISLAVEDARNAA | TGTTTCTGCACGGTTTCACCGGT | C./72 |
| | | | GFLKNVPEVDGTKFGVVGLSMG | AACAAAACCGAATCCGGCCGCC | h |
| | | | GGVAVSLAAGREDV | TTTATACTGACATGGCTCGTGTT | |
| | | | GALVLLSPALDWPELFQRARGFF | CTGTGTTCTGCCGGGTATGCAG | |
| | | | RAEEGYVYWGPNRMRDVYAME | CGCTGCGCTTTGACTTTCGTTGC | |
| | | | TMNFSVMGLAEEIKAP | CATGGGGATTCCCCGCTGCCAT | |
| | | | TLIIHSVDDVVVPISQAKRFYEKL | TCGAGGAATTCCGCATCTCACT | |
| | | | KVEKKFIEIEQGGHVFEDYNVRR | GGCGGTTGAAGATGCGCGTAAT | |
| | | | RIEREVLDWVKRH | GCCGCTGGCTTTCTGAAAAATG | |
| | | | LLEHHHHHH | TTCCTGAAGTTGATGGCACCAA | |
| | | | | ATTCGGCGTGGTTGGTCTGTCT | |
| | | | | ATGGGAGGTGGTGTTGCTGTTT | |
| | | | | CGCTCGCCGCGGGCCGTGAGGA | |
| | | | | TGTAGGTGCTCTGGTACTGCTG | |
| | | | | TCTCCGGCCCTTGATTGGCCGG | |
| | | | | AGCTGTTCCAGCGCGCACGTGG | |
| | | | | CTTCTTCCGCGCGGAAGAAGGT | |
| | | | | TACGTGTACTGGGGTCCGAACC | |
| | | | | GTATGCGTGATGTATACGCAAT | |
| | | | | GGAGACCATGAACTTCAGCGTG | |
| | | | | ATGGGCCTGGCAGAAGAAATTA | |
| | | | | AAGCGCCGACTCTGATCATTCA | |
| | | | | CTCGGTGGATGATGTGGTAGTG | |
| | | | | CCGATCAGTCAGGCTAAACGTT | |
| | | | | TCTACGAAAAACTGAAAGTTGA | |
| | | | | AAAAAAATTTATCGAAATCGAA | |
| | | | | CAGGGCGGCCACGTGTTTGAAG | |
| | | | | ATTACAACGTTCGTCGTCGTAT | |
| | | | | CGAACGTGAAGTTCTGGACTGG | |
| | | | | GTGAAGCGCCATTTA | |
| | 211 | 1 | MLIRPVTFRNMNQQIIGILHTPDN | CTGATTCGTCCGGTTACCTTCCG | 20° |
| | | | IRLNEKVPGILMFHGFTGNKTEA | CAATATGAACCAGCAGATTATT | C./20 |
| | | | HRLFVHVARSLSEH | GGCATCCTTCACACTCCGGACA | hIP |
| | | | GFIVLRFDFRGSGDSDGEFEDMT | ACATCCGTCTGAATGAAAAAGT | TG2xYT |
| | | | LPGEVSDAERALTFLLRQRNVDK | ACCGGGTATCCTGATGTTCCAT | |
| | | | NRIGVIGLSMGGRV | GGCTTCACTGGTAATAAAACTG | |
| | | | AAILASKDRRVKFAVLYSPALGP | AAGCGCACCGCCTGTTTGTGCA | |
| | | | LRDRSLSFMSKEKIERLNSGEAV | CGTGGCTCGTTCTCTGTCCGAA | |
| | | | EFFAEGWYIKKAFF | CATGGTTTCATCGTGCTGCGTTT | |
| | | | ETVDYIVPLDIMDSIKVPVLIVHG | CGACTTCCGCGGAAGCGGTGAT | |
| | | | DKDPLIPVGEAIRA YEKIKGVNE | AGCGATGGTGAATTCGAAGACA | |
| | | | KNELYIVRGGDHT | TGACCCTGCCGGGTGAAGTTAG | |
| | | | FSKKEHTLEVIKKTLDWIRSLGIL | CGACGCAGAGCGCGCGCTGACC | |
| | | | EHHHHHH | TTTCTGTTGCGCCAGCGTAACG | |
| | | | | TTGATAAAAACCGTATTGGTGT | |
| | | | | AATCGGTCTGTCCATGGGTGGC | |
| | | | | CGTGTTGCGGCGATTCTGGCAA | |
| | | | | GCAAGGACCGGCGCGTTAAATT | |
| | | | | CGCTGTCCTGTACAGCCCGGCG | |
| | | | | CTGGGTCCGCTGCGCGATCGTT | |
| | | | | CTCTGTCTTTCATGAGCAAAGA | |
| | | | | AAAAATTGAACGTCTGAACTCC | |
| | | | | GGTGAGGCAGTGGAATTCTTCG | |
| | | | | CTGAAGGTTGGTATATCAAAAA | |
| | | | | AGCATTCTTTGAGACCGTGGAC | |
| | | | | TATATTGTCCCGCTGGACATCA | |
| | | | | TGGATTCCATTAAAGTTCCGGT | |
| | | | | TTTGATCGTTCATGGCGACAAA | |
| | | | | GACCCGCTCATTCCGGTTGGTG | |
| | | | | AGGCTATCCGTGCATACGAAAA | |
| | | | | AATCAAAGGTGTTAACGAGAA | |
| | | | | AAATGAGCTGTACATTGTACGT | |
| | | | | GGCGGTGATCACACCTTCTCCA | |
| | | | | AAAAAGAACACACCCTGGAGG | |
| | | | | TAATCAAGAAAACTTTGGACTG | |
| | | | | GATCCGTAGCCTGGGCATT | |
| | 214 | 1 | MARAAPISPLQRVNFYSAGYRLD | GCGCGCGCAGCGCCGATTTCGC | Auto |
| | | | GLLYTPRHLPAGERRPGVVLLVG | CGCTGCAGCGTGTAAACTTCTA | 28° |
| | | | YTYLKTMVMPDIAKV | CTCTGCAGGTTATCGCTTGGAT | C./24 |
| | | | LNAAGYVALVEDYRGFGESEGP | GGCCTGCTGTATACTCCTCGTC | h |
| | | | RGRLIPLEQVADARAALTFLAEQ | ATCTGCCGGCGGGTGAACGTCG | |
| | | | SMVDPDRLAVIGISL | TCCGGGCGTTGTGCTGCTGGTC | |
| | | | GGAHAITTAALDQRVRAVVAIEP | GGTTACACCTACTTAAAAACCA | |
| | | | PGHGAHWLRSLRRHWEWSQFLS | TGGTGATGCCGGATATCGCTAA | |
| | | | RLTEDRRQRVLSGVS | AGTGCTGAACGCTGCCGGTTAC | |
| | | | STVDPLEIVLPDPESQAFLDQVAA | GTAGCTCTGGTCTTCGATTACC | |
| | | | EFPQMKVTLPLESAEALIEYVPED | GTGGCTTTGGTGAAAGCGAAGG | |
| | | | LAGRIAPRPLLLEHHHHHH | TCCACGTGGTCGTTTGATCCCG | |
| | | | | CTGGAGCAGGTAGCTGACGCGC | |
| | | | | GTGCCGCACTGACCTTCTTGGC | |
| | | | | TGAACAGAGCATGGTCGATCCG | |
| | | | | GACCGTCTGGCAGTCATTGGCA | |
| | | | | TCAGCCTGGGCGGCGCACACGC | |
| | | | | AATCACCACAGCGGCGCTGGAC | |
| | | | | CAACGCGTACGTGCAGTCGTTG | |
| | | | | CGATTGAACCACCGGGTCACGG | |
| | | | | CGCGCACTGGCTGCGTTCCCTT | |
| | | | | CGTCGTCACTGGGAGTGGTCCC | |
| | | | | AGTTCCTGTCTCGCTTGACCGA | |
| | | | | AGATCGTCGTCAGCGCGTTCTG | |
| | | | | TCCGGTGTCAGCAGCACTGTTG | |
| | | | | ACCCACTGGAAATCGTTCTGCC | |
| | | | | AGACCCAGAATCTCAGGCCTTT | |
| | | | | CTGGACCAGGTGGCGGCGGAAT | |
| | | | | TTCCGCAGATGAAAGTGACGCT | |
| | | | | TCCACTGGAATCGGCTGAGGCG | |
| | | | | CTGATTGAATACGTCCCGGAAG | |
| | | | | ACCTGGCAGGTCGTATCGCCCC | |
| | | | | GCGCCCGCTGCTG | |
| Group3 | 301-nSP | 0 | MLLDSRFFFSAFVPLLLASAVVPS | TTGCTGGACAGCCGCTTCTTCTT | Auto + |
| | | | ALRAQPYPVGTRTITYQDPVRNN | TTCCGCTTTCGTACCGCTGCTGC | NaCl |
| | | | RNIQTYLYYPATAAGANQPVAG | TGGCTAGCGCGGTGGTCCCGTC | 25° |
| | | | GQFPVVVVGHGFTMNYAPYAF | CGCACTGCGTGCTCAACCGTAC | C./72 |
| | | | WGNALAESGYIVAIPNTETGFSPS | CCGGTCGGTACTCGTACCATTA | h |
| | | | HSAFAADMAFLVAKLYTENTNS | CTTACCAGGATCCGGTACGTAA | |
| | | | SSPFYQHVQYNSCIIGHSMGGGC | CAACCGCAACATCCAGACGTAC | |
| | | | TYLAAQNNADVSATVTFAAAET | CTGTACTATCCGGCGACCGCAG | |
| | | | NPSATAAAANVNCPSLVFSGSAD | CCGGTGCTAACCAGCCTGTTGC | |
| | | | CITPPAQHQVPMYNALPDCKAY | TGGTGGTCAGTTTCCGGTCGTA | |
| | | | GGSSRVDLQACKLEHHHHHH | GTGGTGGGGCACGGTTTCACTA | |
| | | | | TGAATTACGCGCCGTATGCGTT | |
| | | | | TTGGGGTAACGCGCTGGCTGAG | |
| | | | | TCTGGTTATATCGTAGCTATCCC | |
| | | | | GAACACGGAAACCGGCTTTTCT | |
| | | | | CCGTCCCATAGCGCCTTCGCTG | |
| | | | | CTGATATGGCTTTCCTGGTGGC | |
| | | | | GAAACTGTACACCGAAAACACC | |
| | | | | AACTCCTCCTCCCCTTTTTATCA | |
| | | | | GCATGTTCAGTACAATTCTTGC | |
| | | | | ATTATTGGTCACTCTATGGGTG | |
| | | | | GTGGATGCACTTACCTGGCGGC | |
| | | | | CCAAAACAACGCAGACGTGAG | |
| | | | | CGCTACGGTTACCTTCGCAGCC | |
| | | | | GCAGAAACCAACCCGTCTGCTA | |
| | | | | CCGCGGCTGCAGCAAACGTTAA | |
| | | | | CTGTCCGTCTCTGGTTTTCTCTG | |
| | | | | GTTCCGCCGACTGCATCACCCC | |
| | | | | GCCGGCTCAGCACCAGGTACCG | |
| | | | | ATGTATAACGCTCTGCCGGACT | |
| | | | | GTAAAGCGTACGGCGGTTCTTC | |
| | | | | CCGCGTTGACCTGCAAGCATGC | |
| | | | | AAA | |
| | 305 | 1 | MQVIQQTVTLQKTQLRLTKEGFV | CAAGTAATTCAGCAGACCGTTA | Auto |
| | | | TNYRFPVDFYYPDSPESFPVILISH | CACTGCAAAAAACCCAACTGCG | 28° |
| | | | GFGSVRENFRTLA | CCTGACCAAGGAAGGCTTCGTT | C./24 |
| | | | QHLASHGFLVAVPQHIGSDLQYR | ACCAATTATCGTTTCCCGGTGG | h |
| | | | QELIKGTLSSALSPVEFLARPTDL | ATTTCTACTACCCTGATTCTCCG | |
| | | | STIIDYLQATQNT | GAATCTTTCCCGGTAATTCTGA | |
| | | | GSWQKRANLQQIGVIGDSLGGTT | TCTCTCATGGTTTTGGCTCGGTC | |
| | | | ALTIGGAPLDIPRLQTKCTSDNVI | CGCGAAAACTTCCGCACTCTGG | |
| | | | VNVALILQCQASF | CACAGCATCTGGCCTCTCACGG | |
| | | | LPPSEYNLADSRVKAVIATHPLIS | CTTCCTGGTAGCCGTTCCGCAG | |
| | | | GIFSPDSLAKIQIPVMITAGNFDIIT | CACATCGGCTCGGATCTGCAGT | |
| | | | PLEHHHHHH | ACCGTCAAGAGCTGATCAAAGG | |
| | | | | TACTTTATCCTCCGCACTGTCCC | |
| | | | | CAGTTGAATTTTTGGCGCGTCC | |
| | | | | GACCGACCTGTCTACCATCATT | |
| | | | | GACTATCTGCAGGCGACTCAGA | |
| | | | | ACACCGGCTCCTGGCAGAAGCG | |
| | | | | TGCAAATCTGCAGCAGATCGGC | |
| | | | | GTTATCGGTGATAGTCTGGGCG | |
| | | | | GTACCACTGCTCTGACGATTGG | |
| | | | | TGGTGCACCGCTGGATATTCCG | |
| | | | | CGTCTGCAGACTAAATGTACCT | |
| | | | | CGGACAACGTTATTGTGAACGT | |
| | | | | TGCCCTGATCCTGCAATGCCAG | |
| | | | | GCCTCGTTCCTGCCGCCGAGCG | |
| | | | | AATACAACCTGGCTGATTCCCG | |
| | | | | TGTCAAAGCCGTTATTGCCACG | |
| | | | | CACCCGCTGATCTCAGGCATTT | |
| | | | | TTTCTCCGGACTCTCTGGCGAA | |
| | | | | AATTCAGATCCCAGTGATGATT | |
| | | | | ACCGCGGGCAACTTTGACATCA | |
| | | | | TCACCCCG | |
| | 307 | 2 | MQTVTSMLKDLDAVITQVSEKFP | CAAACCGTGACCAGCATGCTGA | Auto |
| | | | QIDNKRVCLIGHSQGAYVSFLHA | AAGACCTGGACGCGGTAATTAC | 28° |
| | | | TKDERIKCLVSWMGR | TCAGGTTTCAGAAAAATTTCCG | C./23.5 |
| | | | LSDLKEFWSKLWFDEIERKGYIY | CAGATTGACAACAAGCGCGTCT | h |
| | | | EWDYKITKKYVRDSLKYNLSKA | GTCTGATCGGTCACTCTCAGGG | |
| | | | AWRIKVPTLLIYGEL | TGCGTACGTATCCTTCCTGCAT | |
| | | | DDIVPPSEGMKFYRNIKSPKKIVI | GCGACCAAAGATGAACGTATTA | |
| | | | VKDLNHTFSGEKAKKSVIRITLK | AATGCCTGGTCTCCTGGATGGG | |
| | | | WLSKWLKRLDLEHHHHHH | TCGTCTGTCGGACCTGAAAGAA | |
| | | | | TTTTGGTCTAAGCTGTGGTTCG | |
| | | | | ACGAGATCGAACGCAAAGGCT | |
| | | | | ATATCTACGAGTGGGATTACAA | |
| | | | | AATCACCAAGAAATATGTGCGT | |
| | | | | GATAGCCTGAAATACAATCTGT | |
| | | | | CAAAAGCTGCATGGCGTATCAA | |
| | | | | AGTGCCGACCCTGCTGATTTAT | |
| | | | | GGTGAACTGGACGATATCGTGC | |
| | | | | CACCTTCTGAAGGTATGAAATT | |
| | | | | CTACCGCAACATCAAATCTCCG | |
| | | | | AAAAAAATCGTTATTGTAAAGG | |
| | | | | ATCTGAACCACACCTTCTCTGG | |
| | | | | TGAAAAAGCCAAAAAATCCGTT | |
| | | | | ATCCGCATCACTCTGAAATGGC | |
| | | | | TGTCTAAATGGCTCAAGCGCCT | |
| | | | | GGAC | |
| Group4 | 401 | 1 | MANPPGGDPDPGCQTDCNYQRG | GCCAACCCGCCGGGTGGTGACC | 20° |
| | | | PDPTDAYLEAASGPYTVSTIRVSS | CGGACCCTGGCTGCCAGACCGA | C./20 |
| | | | LVPGFGGGTIHYPTN | CTGCAACTATCAGCGCGGTCCG | hIP |
| | | | AGGGKMAGIVVIPGYLSFESSIE | GATCCGACCGACGCTTATCTGG | TG2xYT |
| | | | WWGPRLASHGFVVMTIDTNTIY | AAGCTGCCTCCGGCCCCTACAC | |
| | | | DQPSQRRDQIEAALQ | GGTGTCTACAATCCGCGTATCC | |
| | | | YLVNQSNSSSSPISGMVDSSRLA | TCTCTGGTTCCGGGTTTCGGCG | |
| | | | AVGWSMGGGGTLQLAADGGIK | GCGGTACTATCCACTACCCGAC | |
| | | | AAIALAPWNSSINDFN | GAACGCTGGTGGTGGCAAGATG | |
| | | | RIQVPTLIFACQLDAIAPVALHAS | GCTGGCATCGTTGTGATCCCTG | |
| | | | PFYNRIPNTTPKAFFEMTGGDHW | GTTATCTCTCCTTCGAAAGCTCC | |
| | | | CANGGNIYSALLG | ATCGAATGGTGGGGCCCGCGCC | |
| | | | KYGVSWMKLHLDQDTRYAPFLC | TGGCGTCCCACGGCTTCGTTGT | |
| | | | GPNHAAQTLISEYRGNCPYLEHH | AATGACTATCGACACCAACACC | |
| | | | HHHH | ATCTACGACCAGCCATCTCAGC | |
| | | | | GTCGTGACCAGATCGAAGCAGC | |
| | | | | TCTGCAGTACCTGGTCAACCAG | |
| | | | | TCCAACTCTAGTAGCAGCCCGA | |
| | | | | TTTCTGGGATGGTTGACTCTTCC | |
| | | | | CGCCTCGCGGCAGTAGGTTGGT | |
| | | | | CTATGGGCGGTGGTGGCACCCT | |
| | | | | GCAACTGGCTGCTGACGGTGGT | |
| | | | | ATCAAAGCCGCGATTGCCCTGG | |
| | | | | CTCCGTGGAACAGTTCTATCAA | |
| | | | | TGATTTTAACCGTATTCAGGTA | |
| | | | | CCGACCCTGATCTTCGCTTGTC | |
| | | | | AGCTCGATGCTATCGCTCCAGT | |
| | | | | GGCGCTGCACGCCTCGCCGTTC | |
| | | | | TACAACCGCATCCCTAACACCA | |
| | | | | CGCCGAAAGCGTTTTTCGAAAT | |
| | | | | GACCGGCGGTGACCACTGGTGC | |
| | | | | GCTAACGGCGGTAACATCTATA | |
| | | | | GCGCCCTGCTGGGAAAATATGG | |
| | | | | CGTGTCTTGGATGAAACTGCAC | |
| | | | | CTGGACCAAGATACTCGTTATG | |
| | | | | CTCCGTTCCTGTGCGGCCCGAA | |
| | | | | CCACGCCGCTCAGACCCTGATT | |
| | | | | AGCGAATACCGTGGCAACTGTC | |
| | | | | CTTAC | |
| | 402 | 0 | MAFAITPSPTPTPDPTPNPSPDPGS | GCATTTGCGATCACTCCGTCTC | Auto |
| | | | CSGAECYIRGPNPTVRALEADDG | CGACCCCAACCCCGGATCCGAC | 28° |
| | | | PYSVRTTNVSSFV | CCCGAATCCATCCCCGGATCCG | C./24 |
| | | | SGFGGGTIHYPVGTEGKMGAIAV | GGCTCCTGTTCCGGCGCCGAGT | h |
| | | | IPGYVSYESSIRWWGSRLASWGF | GCTACATCCGCGGTCCTAACCC | |
| | | | VVITIDTNTIYDQP | TACTGTACGTGCCCTGGAAGCA | |
| | | | DSRANQLSAALDYVIAQSNSRNS | GACGATGGTCCGTACTCGGTGC | |
| | | | SISGMVDSNRLGVIGWSMGGGG | GTACCACCAACGTATCTTCCTT | |
| | | | SLKLSTQRTLKAAIP | CGTTTCTGGCTTCGGTGGTGGC | |
| | | | QAPWYSGFNSFNRITTPTLIIACE | ACAATTCACTACCCGGTGGGTA | |
| | | | LDVVAPVGQHASPFYNRIPSSTA | CCGAAGGCAAGATGGGTGCCAT | |
| | | | KAFLEINGGDHFC | CGCCGTGATTCCGGGCTACGTT | |
| | | | ANSGYPNEDILGKYGVSWMKRFI | TCCTACGAATCATCCATCCGTT | |
| | | | DGDRRYDQFLCGPNHESDRSISD | GGTGGGGTAGCCGCCTGGCGTC | |
| | | | YRETCNYLZEHHHHHH | ATGGGGTTTTGTTGTTATTACCA | |
| | | | | TCGACACTAACACCATTTATGA | |
| | | | | TCAACCGGATTCTCGTGCAAAC | |
| | | | | CAGCTGTCAGCCGCTCTGGATT | |
| | | | | ACGTGATCGCTCAAAGCAACTC | |
| | | | | TCGTAACTCGTCCATTTCCGGC | |
| | | | | ATGGTGGACTCCAACCGCCTGG | |
| | | | | GTGTTATCGGCTGGTCTATGGG | |
| | | | | TGGTGGCGGTTCTCTGAAACTG | |
| | | | | TCTACTCAGCGCACGCTGAAAG | |
| | | | | CCGCAATCCCTCAGGCTCCGTG | |
| | | | | GTACTCTGGTTTCAACAGCTTC | |
| | | | | AACCGCATTACTACTCCAACGC | |
| | | | | TCATTATTGCCTGCGAGCTGGA | |
| | | | | CGTTGTAGCTCCTGTAGGTCAG | |
| | | | | CACGCTTCTCCGTTTTACAACC | |
| | | | | GCATTCCGAGCTCCACTGCGAA | |
| | | | | AGCGTTTCTGGAAATCAATGGT | |
| | | | | GGCGACCATTTCTGCGCCAACA | |
| | | | | GCGGCTACCCGAACGAAGACAT | |
| | | | | CCTTGGCAAATATGGCGTTTCT | |
| | | | | TGGATGAAACGCTTTATTGACG | |
| | | | | GTGATCGTCGCTACGACCAGTT | |
| | | | | CCTGTGTGGTCCAAATCACGAA | |
| | | | | TCTGATCGCTCTATCAGCGACT | |
| | | | | ACCGTGAAACCTGTAACTAC | |
| | 403 | 1 | MTTPTPTPEPEPEPPGGCGDCYQ | ACTACCCCAACGCCGACACCTG | Auto |
| | | | RGPDPTVAALEADRGPYSVRTIN | AACCGGAACCGGAACCGCCGG | 28° |
| | | | VSSWVSGFGGGTIHY | GCGGTTGCGGTGACTGTTATCA | C./24 |
| | | | PVGTQGTMGAIA VIPGYVSYENS | GCGTGGGCCTGACCCGACCGTA | h |
| | | | IEWWGGRLASWGFVVITIDTNSI | GCGGCGCTGGAAGCTGACCGCG | |
| | | | YDQPDSRANQLSAA | GTCCGTATTCAGTCCGCACCAT | |
| | | | LDYVIAQSNSSRSAIQGMVDPNR | TAACGTTTCAAGCTGGGTCTCT | |
| | | | LGAIGWSMGGGGTLKLSTDRYL | GGTTTCGGTGGTGGAACTATCC | |
| | | | KAAIPQAPWYSGFNP | ACTACCCGGTAGGTACACAGGG | |
| | | | FDEITTPTLIIACQLDAVAPVAQH | CACCATGGGCGCTATCGCTGTG | |
| | | | ASPFYNEIPNSTAKAFLEIRNGDH | ATCCCGGGTTACGTTTCTTATG | |
| | | | FCANSGYPDEDI | AAAACTCGATCGAATGGTGGGG | |
| | | | LGKYGVAWMKRFIDDDRRYDAF | CGGCCGTCTTGCGTCATGGGGC | |
| | | | LCGPNHEAEWDISEYRDTCNYLE | TTCGTTGTAATTACGATCGACA | |
| | | | HHHHHH | CTAACTCCATCTACGATCAGCC | |
| | | | | GGACTCCCGCGCCAACCAGCTG | |
| | | | | TCTGCTGCTCTGGATTATGTGAT | |
| | | | | CGCGCAGAGCAACTCCAGCCGT | |
| | | | | TCTGCGATCCAGGGCATGGTTG | |
| | | | | ATCCGAACCGCCTGGGTGCAAT | |
| | | | | CGGCTGGTCCATGGGTGGCGGC | |
| | | | | GGTACTCTGAAACTGTCTACGG | |
| | | | | ACCGTTATCTGAAGGCTGCTAT | |
| | | | | TCCGCAGGCGCCATGGTACTCC | |
| | | | | GGCTTTAACCCGTTCGATGAAA | |
| | | | | TCACAACCCCTACCCTCATCAT | |
| | | | | CGCTTGCCAGCTGGATGCTGTC | |
| | | | | GCCCCAGTGGCGCAACACGCTA | |
| | | | | GTCCGTTCTACAACGAAATTCC | |
| | | | | GAACTCTACCGCAAAAGCTTTC | |
| | | | | CTGGAGATCCGTAACGGTGACC | |
| | | | | ACTTCTGCGCAAACAGCGGTTA | |
| | | | | CCCGGATGAGGACATCCTGGGT | |
| | | | | AAATATGGAGTTGCATGGATGA | |
| | | | | AACGTTTCATCGATGACGACCG | |
| | | | | TCGTTATGATGCATTCCTGTGC | |
| | | | | GGTCCGAACCACGAAGCTGAAT | |
| | | | | GGGATATCTCTGAATACCGCGA | |
| | | | | CACTTGCAATTAC | |
| | 405 | 1 | MQADTDTTAVAPAAANPYERGP | CAGGCAGATACCGATACCACTG | 20° |
| | | | APTEASVTAARGPFAIAQVNVPS | CAGTGGCTCCCGCGGCGGCTAA | C./20 |
| | | | GSGAGFNDGTIYYPTD | TCCGTATGAACGCGGCCCGGCT | hIP |
| | | | TSQGTFGAVAVIPGFISPQAVIQW | CCGACTGAAGCGTCTGTAACTG | TG2xYT |
| | | | FGPRLASQGFVVFTLDSNGLADL | CAGCTCGCGGTCCGTTTGCTAT | |
| | | | PDARGRQLLAALD | TGCCCAGGTGAACGTACCGTCT | |
| | | | YLTTQSTVRTRIDPNRLAVMGHS | GGCAGCGGTGCTGGCTTCAACG | |
| | | | MGGGGTLLAAENRPTLKAAIPLA | ATGGCACCATCTACTATCCGAC | |
| | | | PWEPDTSWEGVKVP | TGATACCTCTCAGGGTACCTTT | |
| | | | TMIIGGESDVVAPVSSMAIPDYNS | GGTGCGGTCGCGGTAATCCCGG | |
| | | | LSSAPEKAYLELRSGDHLAPASE | GTTTCATCTCCCCTCAGGCTGTG | |
| | | | SPTVAEYALSWLK | ATCCAGTGGTTCGGTCCGCGCT | |
| | | | RFVDDDTRYDQFLCPGPTPDTDI | TGGCATCTCAGGGCTTCGTAGT | |
| | | | SQYLDTCPNGSLEHHHHHH | CTTCACTCTGGATTCTAACGGT | |
| | | | | CTGGCCGATCTGCCGGATGCGC | |
| | | | | GCGGTCGTCAGCTGCTGGCGGC | |
| | | | | TCTGGACTACCTGACCACCCAG | |
| | | | | TCTACTGTGCGTACCCGTATTG | |
| | | | | ATCCGAATCGCCTGGCTGTCAT | |
| | | | | GGGGCACAGCATGGGTGGCGG | |
| | | | | TGGCACGCTGCTGGCGGCGGAA | |
| | | | | AACCGTCCAACCCTGAAAGCGG | |
| | | | | CCATCCCACTGGCGCCGTGGGA | |
| | | | | ACCGGATACTAGTTGGGAAGGC | |
| | | | | GTGAAAGTACCGACTATGATCA | |
| | | | | TCGGCGGCGAAAGCGATGTCGT | |
| | | | | TGCTCCGGTTTCCAGTATGGCT | |
| | | | | ATTCCGGACTATAACTCCCTGA | |
| | | | | GCTCTGCTCCAGAAAAGGCTTA | |
| | | | | TCTGGAGTTGCGTTCTGGTGAT | |
| | | | | CACCTGGCACCGGCAAGCGAAT | |
| | | | | CTCCTACCGTTGCGGAATACGC | |
| | | | | TTTAAGCTGGCTCAAGCGCTTT | |
| | | | | GTTGATGATGACACTCGTTATG | |
| | | | | ATCAGTTCCTGTGTCCGGGTCC | |
| | | | | TACACCGGATACTGATATCAGC | |
| | | | | CAGTACCTGGATACGTGTCCTA | |
| | | | | ACGGTTCT | |
| | 407 | 2 | MADNPYQRGPDPTRDSVAASRG | GCGGATAACCCGTATCAGCGTG | 20° |
| | | | TFATASTTVGSGNGFGAGFIYYP | GCCCGGATCCGACTCGCGATTC | C./20 |
| | | | TDTSQGTFGAVAIVPG | TGTCGCCGCATCTCGTGGCACC | hIP |
| | | | YTATWAAEGAWMGHWLASFGF | TTCGCTACGGCCTCCACCACCG | TG2xYT |
| | | | VVIGIDTINRNDWDTARGTQLLA | TAGGCTCTGGCAATGGTTTTGG | |
| | | | ALDYLTQRSTVRDRVD | TGCTGGCTTCATCTACTACCCG | |
| | | | ASRLAVMGHSMGGGGAMYAAL | ACTGACACGTCCCAGGGTACAT | |
| | | | QRPSLKAAVGLAPFSPSQNLNGM | TTGGCGCCGTCGCAATCGTGCC | |
| | | | RVPTMLLAGQHDTTTT | GGGTTACACTGCAACCTGGGCA | |
| | | | PASITSLYNGIPAATEKAYLELSG | GCAGAAGGCGCTTGGATGGGTC | |
| | | | AGHGFPTSNNSVMMRKVIPWLKI | ACTGGCTCGCGAGCTTCGGTTT | |
| | | | FVDSDVRYTQFLC | TGTCGTCATCGGCATCGATACC | |
| | | | PLMDNTGIRSYQSTCPLLPGTPTP | ATCAACCGCAACGACTGGGACA | |
| | | | PNRYEAETSPAVCTGTIASNHTG | CTGCGCGTGGTACCCAGCTGCT | |
| | | | YSGTGFCDGNNAT | TGCCGCGCTTGACTACTTGACT | |
| | | | NAYAQFTVNASAAGSMTLRVRF | CAGCGTTCAACCGTTCGTGATC | |
| | | | ANGTTTARPASLIVNGSTVQTPSF | GTGTGGATGCTTCCCGTCTTGC | |
| | | | EGTGAWTTWATKTL | GGTTATGGGCCACTCCATGGGC | |
| | | | TVTLNAGNNTIRFNPTTANGLPN | GGCGGTGGTGCAATGTACGCCG | |
| | | | LDYIEIAAPLEHHHHHH | CACTGCAGCGCCCGAGTCTGAA | |
| | | | | AGCTGCTGTGGGTCTGGCACCG | |
| | | | | TTCTCCCCGTCACAGAACTTGA | |
| | | | | ACGGTATGCGTGTACCGACGAT | |
| | | | | GCTGCTGGCCGGACAACACGAC | |
| | | | | ACCACGACCACGCCGGCGTCCA | |
| | | | | TCACCAGCCTGTACAACGGCAT | |
| | | | | TCCGGCGGCAACTGAAAAAGC | |
| | | | | ATACCTGGAACTGAGCGGTGCG | |
| | | | | GGCCACGGCTTCCCGACCAGCA | |
| | | | | ACAATTCTGTTATGATGCGTAA | |
| | | | | AGTAATTCCGTGGCTGAAAATC | |
| | | | | TTTGTAGATTCAGACGTTCGTT | |
| | | | | ATACGCAGTTTCTGTGTCCGCT | |
| | | | | GATGGATAACACTGGCATCCGT | |
| | | | | AGCTACCAGTCTACCTGTCCTC | |
| | | | | TGCTGCCCGGTACCCCGACTCC | |
| | | | | GCCGAACCGTTACGAAGCCGAG | |
| | | | | ACTTCGCCGGCCGTTTGTACTG | |
| | | | | GTACTATTGCTAGCAACCACAC | |
| | | | | TGGTTATTCCGGTACTGGTTTTT | |
| | | | | GTGACGGTAACAACGCTACCAA | |
| | | | | CGCTTACGCCCAGTTTACCGTT | |
| | | | | AACGCGTCTGCCGCTGGTTCAA | |
| | | | | TGACCCTGCGTGTGCGTTTCGC | |
| | | | | GAACGGTACCACCACCGCTCGC | |
| | | | | CCCGCGAGCCTGATTGTGAACG | |
| | | | | GCAGCACTGTCCAGACCCCGTC | |
| | | | | CTTTGAAGGCACTGGCGCGTGG | |
| | | | | ACCACCTGGGCAACCAAAACAC | |
| | | | | TGACCGTGACCCTGAACGCCGG | |
| | | | | TAACAACACTATCCGTTTCAAC | |
| | | | | CCGACCACCGCGAACGGCCTGC | |
| | | | | CGAACCTTGATTACATCGAAAT | |
| | | | | TGCCGCTCCG | |
| | 409 | 2 | MGDCPATAICRSESPGAYSGNGP | GGTGATTGTCCAGCAACTGCTA | 20° |
| | | | YGSRSYTLSRFQTPGGATVYYPA | TCTGTCGCAGCGAAAGCCCGGG | C./20 |
| | | | NAEPPYAGMVFTPPY | CGCGTACTCCGGTAACGGCCCC | hIP |
| | | | TGTQAMFAAWGPFFASHGFVLV | TATGGTTCTCGCTCCTACACCCT | TG2xYT |
| | | | TMDTSTTLDSVDQRAAQQKEVL | GAGCCGCTTCCAGACGCCGGGT | |
| | | | NALKSENTRSGSPLRG | GGTGCTACCGTGTACTATCCGG | |
| | | | KLDTARLGAVGWSMGGGATWI | CGAACGCAGAACCGCCGTACGC | |
| | | | NSAEYSGLKTAMSLAGHNLTAV | TGGTATGGTCTTTACCCCGCCG | |
| | | | DIDSKGYNTRVPTLLFN | TATACCGGCACTCAGGCGATGT | |
| | | | GAQDLTYLGGLGQSDGVYNNIP | TCGCTGCTTGGGGCCCATTCTTC | |
| | | | AGIPKVFYEVSSAGHFDWGSPTA | GCGTCTCACGGCTTCGTTCTGG | |
| | | | ANRSVASLALAFHKA | TTACCATGGACACGAGCACCAC | |
| | | | YLDGDTRWLQYITRPSSDVTTW | ACTGGACTCCGTCGACCAGCGT | |
| | | | RTANIRLEHHHHHH | GCTGCTCAGCAGAAAGAAGTAC | |
| | | | | TGAACGCACTGAAATCTGAGAA | |
| | | | | CACCCGTTCCGGCTCTCCACTG | |
| | | | | CGCGGTAAACTGGATACCGCAC | |
| | | | | GTCTGGGCGCTGTTGGCTGGTC | |
| | | | | CATGGGTGGTGGCGCAACTTGG | |
| | | | | ATCAATAGCGCAGAATACTCCG | |
| | | | | GCCTGAAAACCGCTATGTCTCT | |
| | | | | GGCTGGTCACAACCTGACGGCA | |
| | | | | GTTGATATTGATAGCAAGGGCT | |
| | | | | ATAATACCCGTGTGCCGACCCT | |
| | | | | GCTGTTCAACGGTGCACAGGAT | |
| | | | | CTGACTTACCTGGGCGGTTTGG | |
| | | | | GCCAGTCTGATGGCGTATACAA | |
| | | | | CAACATCCCGGCGGGAATCCCG | |
| | | | | AAAGTTTTTTATGAAGTCAGCA | |
| | | | | GCGCGGGCCACTTTGATTGGGG | |
| | | | | TTCCCCGACTGCGGCCAACCGT | |
| | | | | TCTGTGGCGTCTCTGGCGCTTG | |
| | | | | CCTTCCACAAAGCATACCTGGA | |
| | | | | TGGCGACACCCGTTGGCTGCAG | |
| | | | | TACATTACTCGTCCGAGCAGCG | |
| | | | | ATGTTACTACTTGGCGTACCGC | |
| | | | | GAACATTCGT | |
| | 410 | 0 | MSQVPPTDPQDAPLGECPATALC | TCCCAAGTCCCGCCAACGGATC | Auto |
| | | | RSEAPGSYSGNGPYGYRSYSLSR | CTCAGGACGCGCCGTTGGGCGA | 28° |
| | | | LQTPGGATVYYPANA | ATGCCCTGCTACCGCCTTGTGT | C./24 |
| | | | EPPYSGLVFTPPYTGVQFMYAA | CGTTCAGAAGCGCCGGGTTCTT | h |
| | | | WGPFFASHGIVLVTMDTTTTLDT | ACAGCGGCAACGGTCCGTACGG | |
| | | | VDQRARQQKTVLDVL | TTATCGCAGCTATTCCCTGTCTC | |
| | | | KGENNRAASPLRGKLDTSRIGAV | GTCTGCAAACCCCGGGCGGCGC | |
| | | | GWSMGGGATWINAAEYAGLKT | AACCGTTTATTATCCGGCAAAC | |
| | | | AMSLAGHNLSAIDPNA | GCGGAGCCACCGTACTCGGGTC | |
| | | | RGYNTRVPTLLFNGALDATYLG | TCGTTTTCACGCCGCCGTACAC | |
| | | | GLGQSDGVYNAIPAGIPKVFYEV | CGGCGTGCAATTCATGTACGCC | |
| | | | ASAGHFDWGSPTAAN | GCGTGGGGTCCGTTTTTTGCGT | |
| | | | RDVAGIALAFHKAFLDGDTRWV | CCCACGGCATCGTACTGGTGAC | |
| | | | DYIRRPSRDVATWRTAYLPDLEH | TATGGATACCACTACTACCCTG | |
| | | | HHHHH | GACACTGTTGATCAACGCGCAC | |
| | | | | GTCAACAGAAAACTGTACTGGA | |
| | | | | TGTTCTGAAAGGCGAAAACAAT | |
| | | | | CGTGCAGCATCGCCGCTGCGCG | |
| | | | | GTAAACTGGATACCTCACGTAT | |
| | | | | TGGTGCTGTTGGCTGGTCCATG | |
| | | | | GGTGGAGGCGCGACCTGGATCA | |
| | | | | ATGCAGCTGAATATGCAGGTCT | |
| | | | | GAAAACCGCGATGTCTTTGGCT | |
| | | | | GGCCATAACCTGTCCGCTATCG | |
| | | | | ATCCGAATGCGCGTGGCTACAA | |
| | | | | CACTCGCGTGCCGACCTTACTG | |
| | | | | TTCAACGGTGCACTGGACGCGA | |
| | | | | CCTACCTGGGCGGTCTGGGTCA | |
| | | | | GAGCGATGGGGTGTATAATGCA | |
| | | | | ATCCCGGCGGGCATCCCTAAGG | |
| | | | | TATTCTACGAAGTTGCCAGCGC | |
| | | | | GGGGCATTTCGATTGGGGTTCC | |
| | | | | CCTACCGCCGCTAACCGTGATG | |
| | | | | TAGCGGGTATTGCACTGGCGTT | |
| | | | | CCACAAAGCATTCCTGGACGGC | |
| | | | | GACACCCGCTGGGTCGATTACA | |
| | | | | TCCGCCGCCCTTCTCGTGACGTT | |
| | | | | GCAACTTGGCGCACCGCATACC | |
| | | | | TGCCAGAC | |
| | 412 | 1 | MSQVPPTPPTDDPMGDCPSTAIC | TCCCAGGTTCCGCCGACCCCGC | 20° |
| | | | RGEAPGSYSGNGPYGSRSYTLSR | CGACCGATGATCCGATGGGTGA | C./20 |
| | | | FQTPGGATVYYPSNA | TTGCCCGTCTACAGCTATCTGC | hIP |
| | | | EPPYSGLVFTPPYTGTQAMFRAW | CGAGGCGAGGCGCCGGGTAGC | TG2xYT |
| | | | GPFFASHGIVLVTMDTSTTVDTV | TATTCTGGTAACGGCCCGTATG | |
| | | | DQRASQQKRVLDVL | GTTCCCGGAGCTACACCCTGTC | |
| | | | KQENTRSGSPLRGKLDTSRLGAV | TCGTTTCCAGACCCCGGGCGGC | |
| | | | GWSMGGGATWINSAEYNGLKT | GCAACCGTATACTACCCGTCTA | |
| | | | AMSLAGHNMTAIDLDS | ACGCCGAACCACCGTACAGCGG | |
| | | | KGGNTRVPTLLFNGALDLTMLG | TCTGGTTTTCACTCCGCCGTACA | |
| | | | GLGQSIGVYNAIPRGIPKVIYEVA | CCGGTACTCAGGCTATGTTTCG | |
| | | | SAGHFDWGSPTAAN | CGCATGGGGCCCATTTTTTGCA | |
| | | | RSVAGIALAFHKTFLDGDTRWVS | TCTCACGGTATCGTTCTGGTAA | |
| | | | YIKRPSSDVATWRTENLPQLEHH | CCATGGACACGTCCACTACAGT | |
| | | | HHHH | GGACACCGTTGATCAGCGTGCG | |
| | | | | AGCCAGCAGAAACGCGTACTG | |
| | | | | GACGTTCTGAAACAGGAAAAC | |
| | | | | ACGCGTTCGGGCTCTCCGCTCC | |
| | | | | GTGGTAAGCTGGACACTTCCCG | |
| | | | | TCTGGGTGCCGTGGGCTGGAGT | |
| | | | | ATGGGTGGCGGAGCTACCTGGA | |
| | | | | TCAACTCTGCGGAGTACAACGG | |
| | | | | TCTCAAAACGGCTATGAGCCTC | |
| | | | | GCAGGTCACAATATGACCGCTA | |
| | | | | TCGATCTGGACAGCAAAGGTGG | |
| | | | | TAACACCCGTGTTCCGACCCTC | |
| | | | | CTGTTCAACGGCGCGCTGGACC | |
| | | | | TGACCATGCTGGGTGGCCTGGG | |
| | | | | CCAGTCTATCGGTGTTTACAAC | |
| | | | | GCTATCCCGCGCGGTATTCCGA | |
| | | | | AAGTTATCTACGAAGTTGCCAG | |
| | | | | CGCTGGGCACTTCGACTGGGGT | |
| | | | | TCCCCAACCGCAGCGAATCGTT | |
| | | | | CCGTTGCGGGTATCGCACTGGC | |
| | | | | GTTCCACAAAACGTTTCTGGAT | |
| | | | | GGCGACACCCGTTGGGTTTCCT | |
| | | | | ACATCAAACGTCCATCCTCCGA | |
| | | | | TGTGGCTACCTGGCGTACCGAA | |
| | | | | AACCTGCCGCAG | |
| Group5 | 501 | 3 | MSNPYQRGPNPTRSALTADGPFS | TCCAACCCATACCAACGTGGTC | Auto |
| | | | VATYTVSRLSVSGFGGGVIYYPT | CGAACCCGACCCGTTCTGCCTT | 28° |
| | | | GTSLTFGGIAMSPGY | GACCGCCGACGGTCCTTTCTCA | C./24 |
| | | | TADASSLAWLGRRLASHGFVVL | GTTGCTACCTATACTGTTAGCC | h |
| | | | VINTNSRFDYPDSRASQLSAALN | GTTTATCCGTATCTGGTTTCGGT | |
| | | | YLRTSSPSAVRARLD | GGCGGCGTTATTTACTATCCGA | |
| | | | ANRLAVAGHSMGGGGTLRIAEQ | CTGGTACCTCCCTGACCTTCGG | |
| | | | NPSLKAAVPLTPWHTDKTFNTSV | CGGCATCGCGATGAGCCCGGGT | |
| | | | PVLIVGAEADTVAPV | TACACCGCCGATGCTTCCAGCC | |
| | | | SQHAIPFYQNLPSTTPKVYVELD | TGGCGTGGCTGGGTCGCCGTCT | |
| | | | NASHFAPNSNNAAISVYTISWMK | GGCTTCCCACGGCTTTGTAGTT | |
| | | | LWVDNDTRYRQFLC | CTGGTCATTAACACCAACTCAC | |
| | | | NVNDPALSDFRTNNRHCQLEHH | GTTTCGACTACCCGGACTCTCG | |
| | | | HHHH | TGCGTCTCAGCTGTCCGCCGCT | |
| | | | | CTGAACTATCTGCGTACGTCAT | |
| | | | | CTCCTTCTGCAGTTCGCGCTCGT | |
| | | | | CTGGATGCTAATCGTCTGGCTG | |
| | | | | TAGCCGGCCACAGCATGGGTGG | |
| | | | | TGGTGGTACGCTGCGCATCGCC | |
| | | | | GAACAGAACCCGTCTCTGAAAG | |
| | | | | CTGCGGTTCCGTTGACTCCGTG | |
| | | | | GCATACCGATAAAACTTTTAAC | |
| | | | | ACTTCCGTGCCGGTTCTCATTGT | |
| | | | | AGGTGCCGAAGCGGATACTGTC | |
| | | | | GCACCAGTCTCCCAGCACGCGA | |
| | | | | TCCCGTTCTACCAGAACCTGCC | |
| | | | | ATCCACTACCCCTAAAGTGTAT | |
| | | | | GTAGAACTGGATAACGCATCTC | |
| | | | | ACTTTGCGCCTAACTCTAACAA | |
| | | | | CGCGGCTATCAGCGTGTACACC | |
| | | | | ATCTCGTGGATGAAACTCTGGG | |
| | | | | TTGATAACGACACTCGTTACCG | |
| | | | | CCAGTTCCTGTGTAACGTTAAC | |
| | | | | GATCCAGCCCTGTCAGATTTTC | |
| | | | | GTACGAACAACCGACACTGTCA | |
| | | | | A | |
| | 503 | 1 | MESPYERGPDPTSASVLDNGTFS | GAGAGTCCGTACGAACGTGGTC | 20° |
| | | | LSSTSVSSLVTGFGGGTIYYPTST | CGGACCCGACTTCTGCATCCGT | C./20 |
| | | | TQGTFGGVVLAPGY | TCTGGATAATGGAACCTTTTCA | hIP |
| | | | TASSSSYSSVARRVASHGFVVFAI | CTGTCCTCCACGTCCGTGTCTTC | TG2xYT |
| | | | DTNSRYDQPDSRGSQILAAVSYL | TCTTGTGACGGGTTTCGGTGGC | |
| | | | KNSASSTVASRLD | GGCACCATTTATTATCCGACCT | |
| | | | ETRIAVSGHSMGGGGTLAAANQ | CCACCACTCAGGGCACGTTTGG | |
| | | | DSSIKAAVALQPWHTDKTWPGIQ | CGGCGTAGTTTTAGCACCGGGC | |
| | | | IPTMIIGAENDSVAP | TACACTGCGAGCAGCTCCTCTT | |
| | | | VASHSIPFYTSMTGAREKAYGEI | ATTCTAGCGTGGCCCGCCGCGT | |
| | | | NNGDHFIANTDDDWQGRLFVTW | GGCATCTCACGGCTTTGTGGTC | |
| | | | LKRYVDDDTRYSQFL | TTCGCGATTGATACTAATTCGC | |
| | | | CPAPSSIYLSDYRNTCPDLEHHH | GCTACGATCAGCCGGATAGCCG | |
| | | | HHH | TGGTAGCCAGATTCTGGCGGCT | |
| | | | | GTATCCTACCTGAAAAACTCTG | |
| | | | | CGTCGTCCACCGTGGCCTCCCG | |
| | | | | CTTGGATGAGACCCGTATCGCG | |
| | | | | GTTAGCGGTCATTCTATGGGCG | |
| | | | | GGGGCGGCACCCTGGCAGCCGC | |
| | | | | CAACCAAGATTCTTCCATCAAA | |
| | | | | GCTGCGGTCGCACTGCAACCGT | |
| | | | | GGCACACGGATAAGACGTGGC | |
| | | | | CGGGCATCCAAATCCCGACTAT | |
| | | | | GATTATCGGCGCTGAAAACGAC | |
| | | | | TCCGTTGCGCCGGTCGCCAGCC | |
| | | | | ACTCTATTCCGTTTTATACTTCT | |
| | | | | ATGACCGGCGCTCGCGAAAAG | |
| | | | | GCGTATGGTGAAATCAACAACG | |
| | | | | GTGATCACTTCATCGCTAACAC | |
| | | | | CGATGACGACTGGCAGGGCCGT | |
| | | | | TTGTTCGTTACCTGGCTGAAAC | |
| | | | | GCTATGTCGATGATGATACGCG | |
| | | | | TTACTCCCAGTTTCTGTGCCCGG | |
| | | | | CGCCGTCCTCTATCTACTTGTCT | |
| | | | | GATTATCGCAACACCTGTCCGG | |
| | | | | AT | |
| | 504 | 2 | MQAQYQKGPDPTASALERNGPF | CAGGCGCAGTACCAGAAAGGT | 20° |
| | | | AIRSTSVSRTSVSGFGGGRLYYPT | CCGGATCCGACTGCTTCTGCTC | C./19 |
| | | | ASGTYGAIAVSPGFT | TGGAGCGCAACGGTCCGTTCGC | hIP |
| | | | GTSSTMTFWGERLASHGFVVLVI | TATCCGTTCAACCAGCGTTAGC | TG2xYT |
| | | | DTITLYDQPDSRARQLKAALDYL | CGTACTAGCGTAAGCGGCTTTG | |
| | | | ATQNGRSSSPIYRK | GTGGTGGCCGTCTGTACTACCC | |
| | | | VDTSRRAVAGHSMGGGGSLLAA | GACGGCCAGCGGCACGTATGGT | |
| | | | RDNPSYKAAIPMAPWNTSSTAFR | GCGATTGCCGTTAGCCCTGGTT | |
| | | | TVSVPTMIFGCQDDS | TTACCGGCACTAGCTCTACTAT | |
| | | | IAPVFSHAIPFYNAIPNSTRKNYV | GACCTTTTGGGGTGAACGTCTG | |
| | | | EIRNDDHFCVMNGGGHDATLGK | GCCTCTCACGGCTTCGTAGTAC | |
| | | | LGISWMKRFVDNDT | TTGTAATCGATACAATCACTCT | |
| | | | RYSPFVCGAEYNRVVSSYEVSRS | GTACGATCAGCCGGACTCCCGC | |
| | | | YNNCPYLEHHHHHH | GCACGCCAGCTGAAAGCAGCA | |
| | | | | CTGGACTACCTGGCCACCCAGA | |
| | | | | ACGGTCGCTCCTCATCTCCGAT | |
| | | | | CTATCGTAAAGTCGACACTTCT | |
| | | | | CGTCGTGCGGTTGCCGGCCACA | |
| | | | | GCATGGGTGGTGGCGGCAGTCT | |
| | | | | GCTGGCAGCACGTGACAATCCA | |
| | | | | TCTTACAAAGCCGCGATCCCAA | |
| | | | | TGGCGCCGTGGAACACCTCCTC | |
| | | | | TACCGCCTTTCGTACCGTTTCTG | |
| | | | | TCCCGACCATGATCTTCGGCTG | |
| | | | | TCAGGATGACTCTATCGCCCCA | |
| | | | | GTATTCTCTCATGCTATCCCGTT | |
| | | | | CTACAACGCGATCCCGAACAGC | |
| | | | | ACGCGCAAAAACTACGTTGAAA | |
| | | | | TCCGTAACGACGACCACTTCTG | |
| | | | | TGTGATGAACGGCGGTGGCCAC | |
| | | | | GATGCAACTCTGGGTAAATTGG | |
| | | | | GCATCTCTTGGATGAAACGCTT | |
| | | | | CGTGGACAATGATACCCGTTAC | |
| | | | | AGCCCGTTCGTGTGTGGTGCGG | |
| | | | | AGTACAACCGTGTTGTTTCATC | |
| | | | | TTACGAAGTGTCCCGTTCTTAT | |
| | | | | AACAACTGTCCGTAT | |
| Group6 | 601 | 3 | MAANPYQRGPDPTESLLRAARG | GCTGCGAATCCGTACCAACGTG | Auto |
| | | | PFAVSEQSVSRLSVSGFGGGRIYY | GCCCGGATCCAACCGAATCGCT | 28° |
| | | | PTTTSQGTFGAIAIS | GCTGCGCGCCGCTCGCGGTCCG | C./23.5 |
| | | | PGFTASWSSLAWLGPRLASHGFV | TTCGCCGTTTCAGAACAATCTG | h |
| | | | VIGIETNTRLDQPDSRGRQLLAAL | TTTCTCGTTTATCTGTCTCCGGT | |
| | | | DYLTQRSSVRNRV | TTTGGTGGTGGTCGTATCTACT | |
| | | | DASRLAVAGHSMGGGGTLEAAK | ATCCGACCACTACGTCCCAGGG | |
| | | | SRTSLKAAIPIAPWNLDKTWPEV | TACGTTTGGCGCTATCGCTATT | |
| | | | RTPTLIIGGELDSIA | AGCCCGGGTTTTACCGCATCAT | |
| | | | PVATHSIPFYNSLTNAREKAYLEL | GGAGCTCGCTCGCTTGGCTGGG | |
| | | | NNASHFFPQFSNDTMAKFMISW | CCCGCGCCTGGCGAGTCATGGT | |
| | | | MKRFIDDDTRYDQF | TTCGTAGTTATCGGTATTGAAA | |
| | | | LCPPPRAIGDISDYRDTCPHTLEH | CCAACACCCGCCTGGACCAGCC | |
| | | | HHHHH | GGATTCCCGTGGCCGTCAGCTG | |
| | | | | CTGGCTGCTCTGGACTACCTGA | |
| | | | | CCCAGCGTTCCTCTGTGCGCAA | |
| | | | | CCGTGTTGACGCGTCTCGCCTG | |
| | | | | GCGGTCGCAGGTCACTCCATGG | |
| | | | | GTGGTGGCGGCACTCTGGAAGC | |
| | | | | GGCAAAGAGCCGTACCAGCCTG | |
| | | | | AAAGCTGCAATCCCGATTGCAC | |
| | | | | CGTGGAACCTGGACAAAACTTG | |
| | | | | GCCGGAAGTTCGCACCCCGACC | |
| | | | | CTGATTATTGGCGGTGAATTGG | |
| | | | | ACAGCATTGCTCCGGTCGCTAC | |
| | | | | CCATAGCATTCCGTTTTACAAC | |
| | | | | TCTCTGACCAATGCACGTGAAA | |
| | | | | AAGCTTATCTGGAACTGAACAA | |
| | | | | CGCGTCTCACTTTTTTCCTCAGT | |
| | | | | TTTCCAACGATACCATGGCTAA | |
| | | | | ATTCATGATCTCTTGGATGAAA | |
| | | | | CGCTTCATCGATGACGATACGC | |
| | | | | GTTATGACCAGTTCCTGTGCCC | |
| | | | | GCCGCCGCGTGCTATCGGTGAT | |
| | | | | ATTTCGGACTACCGTGATACTT | |
| | | | | GTCCGCACACC | |
| | 602 | 2 | MAANPYQRGPNPTEASITAARGP | GCTGCTAACCCGTATCAGCGTG | Auto |
| | | | FNTAEITVSRLSVSGFGGGKIYYP | GCCCGAACCCCACTGAGGCGAG | 28° |
| | | | TTTSEGTFGAIAIS | CATCACTGCCGCGCGCGGTCCA | C./23.5 |
| | | | PGFTAYWSSLEWLGHRLASQGF | TTCAATACTGCGGAAATTACCG | h |
| | | | VVIGIETNTTLDQPDQRGQQLLA | TTTCTCGCCTGTCCGTATCCGGT | |
| | | | ALDYLTQRSAVRDRV | TTCGGTGGTGGCAAAATCTACT | |
| | | | DASRLAVAGHSMGGGGSLEAAK | ATCCAACGACCACCTCGGAAGG | |
| | | | ARTSLKAAIPLAPWNLDKTWPEV | TACCTTCGGTGCTATCGCAATTT | |
| | | | RTPTLIIGGELDAVA | CTCCGGGTTTCACCGCATACTG | |
| | | | PVATHSIPFYNSLSNAPEKAYLEL | GAGCTCTCTCGAATGGCTGGGC | |
| | | | DNASHFFPNITNTQMAKYMIAW | CACCGTCTGGCTAGCCAGGGCT | |
| | | | MKRFIDDDTRYTQF | TTGTTGTAATCGGTATCGAAAC | |
| | | | LCPPPSTGLLSDFSDARFTCPMLE | TAACACTACTTTAGACCAGCCG | |
| | | | HHHHHH | GACCAGCGTGGCCAGCAGCTGC | |
| | | | | TCGCTGCGCTGGACTATCTGAC | |
| | | | | CCAGCGCTCAGCAGTTCGTGAT | |
| | | | | CGTGTTGATGCATCTCGTCTGG | |
| | | | | CGGTAGCGGGTCATTCGATGGG | |
| | | | | CGGTGGTGGTTCTCTGGAAGCT | |
| | | | | GCAAAAGCTCGTACGAGTCTGA | |
| | | | | AAGCGGCGATTCCTCTGGCACC | |
| | | | | CTGGAACCTGGACAAAACTTGG | |
| | | | | CCGGAGGTGCGCACTCCGACCC | |
| | | | | TTATTATTGGTGGTGAACTGGA | |
| | | | | CGCCGTCGCGCCGGTGGCGACC | |
| | | | | CACTCTATCCCGTTCTACAACA | |
| | | | | GCCTGAGCAACGCTCCGGAGAA | |
| | | | | AGCCTACCTCGAACTGGATAAC | |
| | | | | GCGTCTCACTTCTTTCCGAATAT | |
| | | | | TACCAACACTCAGATGGCGAAA | |
| | | | | TACATGATCGCATGGATGAAAC | |
| | | | | GTTTCATCGATGACGATACCCG | |
| | | | | TTACACCCAGTTCCTGTGCCCG | |
| | | | | CCTCCGTCTACCGGCCTGCTGA | |
| | | | | GCGACTTTTCAGATGCACGTTT | |
| | | | | TACATGCCCGATG | |
| | 605 | 0 | MAADNPYERGPAPTESSIEALRG | GCCGCGGACAATCCGTACGAAC | Auto |
| | | | PYAVSQTSVSRLAATGFGGGTIY | GTGGCCCAGCGCCGACCGAATC | 28° |
| | | | YPTSTADGTFGAVAI | CTCGATCGAAGCACTGCGCGGT | C./23.5 |
| | | | SPGFTALESSISWLGPRLASQGFV | CCTTACGCTGTTTCCCAGACCTC | h |
| | | | VFTIDTLTTVDQPGSRGDQLLAA | TGTGTCTCGGCTGGCTGCAACT | |
| | | | LDYLTQRSSVRGR | GGCTTCGGCGGCGGCACGATTT | |
| | | | IDSSRLGVMGHSMGGGGSLEAA | ACTATCCGACCAGCACCGCGGA | |
| | | | KTRPSLKAAIPMTPWNLDKTWPE | CGGCACGTTTGGTGCTGTGGCA | |
| | | | LRTPTLIFGADADTI | ATCAGCCCGGGTTTCACTGCCC | |
| | | | APVATHAKPFYNTLPSSLDRTYIE | TGGAAAGCTCTATTTCCTGGTT | |
| | | | LNNATHFAPNTSNTTIAKYSISWL | GGGCCCGCGTCTGGCGTCTCAA | |
| | | | KRFIDKDTRYEQ | GGCTTCGTGGTGTTTACGATCG | |
| | | | FLCPLPQRSLTIDEAQGNCPHTSL | ACACCCTGACCACTGTGGACCA | |
| | | | EHHHHHH | GCCGGGTTCCCGTGGTGACCAG | |
| | | | | CTCCTGGCCGCGCTTGATTACC | |
| | | | | TCACTCAGCGCTCTTCTGTTCGC | |
| | | | | GGTCGCATCGATTCCTCCCGTC | |
| | | | | TGGGCGTTATGGGTCACTCAAT | |
| | | | | GGGTGGCGGCGGTTCCTTGGAA | |
| | | | | GCTGCTAAAACCCGTCCGAGCC | |
| | | | | TCAAAGCTGCTATTCCTATGAC | |
| | | | | CCCTTGGAACCTGGATAAGACA | |
| | | | | TGGCCTGAGCTGAGGACCCCTA | |
| | | | | CTCTGATTTTTGGCGCGGATGC | |
| | | | | TGACACCATCGCGCCGGTGGCG | |
| | | | | ACTCACGCGAAACCTTTCTATA | |
| | | | | ATACTCTGCCTTCTTCCCTTGAC | |
| | | | | CGTACTTACATCGAACTGAACA | |
| | | | | ACGCTACCCACTTTGCTCCTAA | |
| | | | | CACGTCTAACACGACCATCGCT | |
| | | | | AAATACTCCATCTCGTGGCTGA | |
| | | | | AACGTTTCATCGACAAAGATAC | |
| | | | | CCGCTATGAACAGTTCCTGTGT | |
| | | | | CCGCTGCCTCAGCGTAGCCTTA | |
| | | | | CCATTGACGAAGCGCAGGGCA | |
| | | | | ACTGTCCGCACACCTCC | |
| | 606 | 2 | MSNPYERGPAPTESSVTAVRGYF | TCCAACCCGTACGAACGCGGCC | Auto |
| | | | DTDTDTVSSLVSGFGGGTIYYPT | CGGCACCAACCGAATCTTCCGT | 28° |
| | | | DTSEGTFGGVVIAPG | TACCGCGGTGCGCGGTTATTTC | C./24 |
| | | | YTASQSSMAWMGHRIASQGFVV | GACACCGATACTGACACCGTTT | h |
| | | | FTIDTITRYDQPDSRGRQIEAALD | CGTCTCTGGTTTCCGGTTTCGGC | |
| | | | YLVEDSDVADRVDG | GGGGGTACGATTTACTATCCGA | |
| | | | NRLAVMGHSMGGGGTLAAAEN | CTGACACTAGTGAAGGTACTTT | |
| | | | RPELRAAIPLTPWHLQKNWSDVE | CGGCGGCGTGGTGATCGCGCCG | |
| | | | VPTMIIGAENDTVASV | GGCTACACCGCTTCACAGTCAT | |
| | | | RTHSIPFYESLDEDLERAYLELDG | CTATGGCATGGATGGGCCACCG | |
| | | | ASHFAPNISNTVIAKYSISWLKRF | TATTGCGTCTCAGGGCTTCGTT | |
| | | | VDEDERYEQFLC | GTATTTACTATCGATACGATTA | |
| | | | PPPDTGLFSDFSDYRDSCPHTTLE | CGCGTTATGATCAGCCGGATTC | |
| | | | HHHHHH | ACGTGGTCGTCAGATCGAAGCA | |
| | | | | GCTCTGGACTACCTGGTGGAAG | |
| | | | | ATTCTGATGTAGCCGACCGTGT | |
| | | | | TGACGGCAACCGCCTGGCCGTT | |
| | | | | ATGGGTCACTCTATGGGTGGTG | |
| | | | | GTGGCACCCTGGCTGCAGCCGA | |
| | | | | AAACCGCCCGGAACTGCGTGCA | |
| | | | | GCTATCCCGCTGACCCCGTGGC | |
| | | | | ACCTGCAGAAGAATTGGTCTGA | |
| | | | | TGTTGAAGTGCCGACGATGATT | |
| | | | | ATCGGCGCTGAAAATGATACCG | |
| | | | | TGGCGAGCGTACGTACCCATTC | |
| | | | | CATCCCGTTTTACGAATCTCTG | |
| | | | | GATGAAGATCTGGAACGCGCGT | |
| | | | | ACTTGGAACTGGATGGTGCTTC | |
| | | | | CCATTTCGCTCCGAACATTTCTA | |
| | | | | ACACCGTTATCGCAAAATATAG | |
| | | | | CATCTCCTGGCTGAAACGTTTC | |
| | | | | GTTGATGAAGATGAACGTTACG | |
| | | | | AACAATTCCTGTGTCCGCCGCC | |
| | | | | GGACACTGGGCTGTTTTCAGAC | |
| | | | | TTCTCCGATTACCGCGACTCTTG | |
| | | | | CCCACATACCACC | |
| | 608 | 0 | MADNPYARGPEPTTASVEAARG | GCGGATAACCCATATGCGCGCG | 20° |
| | | | PFAVAQTSVSRYAVSGFGGGTV | GTCCAGAACCGACCACCGCTTC | C./20 |
| | | | YYPTTTTAGTFGAVAVS | TGTTGAGGCGGCTCGTGGTCCG | hIP |
| | | | PGYTARQSSIAWLGPRLASQGFV | TTTGCTGTTGCGCAGACGTCCG | TG2xYT |
| | | | VITIDTLSTYDQPASRGDQLRAAL | TTTCCCGTTACGCTGTTAGTGGC | |
| | | | AYLTQRSSVRARI | TTTGGTGGCGGTACCGTATACT | |
| | | | DPTRLAVVGHSMGGGGALEAAK | ACCCGACGACCACCACTGCAGG | |
| | | | DDPSLQAAVPLTGWNLDKTWPE | TACCTTCGGTGCGGTAGCAGTG | |
| | | | VRTPTLVIGAEDDGVA | AGCCCGGGTTATACCGCTCGTC | |
| | | | PVRSHSEPFYASLPATLDKAYLE | AGAGCTCCATTGCGTGGCTGGG | |
| | | | LRGAGHLAPTVSNTTIATYTLSW | TCCACGTCTTGCTTCACAGGGT | |
| | | | LKRFVDDDLRYDRF | TTTGTGGTGATTACGATCGACA | |
| | | | LCPAPATSTAIAEYRSTCPYLEHH | CCCTGTCGACCTACGACCAGCC | |
| | | | HHHH | GGCGTCTCGTGGTGATCAGCTG | |
| | | | | CGTGCAGCGCTGGCATACCTGA | |
| | | | | CTCAGCGTTCTAGCGTTCGCGC | |
| | | | | CCGCATCGACCCGACGCGTCTA | |
| | | | | GCGGTAGTTGGCCACTCCATGG | |
| | | | | GTGGTGGTGGCGCGCTGGAAGC | |
| | | | | GGCCAAAGACGATCCGTCACTG | |
| | | | | CAGGCGGCAGTGCCGCTGACCG | |
| | | | | GCTGGAACCTTGATAAAACTTG | |
| | | | | GCCGGAAGTGCGCACACCGACC | |
| | | | | CTTGTAATCGGCGCCGAAGATG | |
| | | | | ACGGCGTAGCGCCGGTACGTTC | |
| | | | | CCACTCTGAACCGTTTTACGCA | |
| | | | | TCTCTGCCAGCCACTCTCGATA | |
| | | | | AGGCATACCTGGAATTACGCGG | |
| | | | | CGCTGGCCACCTGGCGCCTACC | |
| | | | | GTTTCCAACACTACGATCGCCA | |
| | | | | CCTATACCCTCTCTTGGCTGAA | |
| | | | | ACGTTTCGTTGACGACGACCTG | |
| | | | | CGCTATGACCGTTTCCTGTGTCC | |
| | | | | GGCTCCGGCTACAAGCACTGCA | |
| | | | | ATTGCGGAATACCGTTCTACGT | |
| | | | | GCCCGTAT | |
| | 611 | 2 | MAEPADVHGPDPTEESITAPRGP | GCCGAACCCGCTGACGTACACG | 20° |
| | | | FEVDEESVSRLSVSGFGGGTIYYP | GCCCGGACCCAACCGAAGAATC | C./20 |
| | | | TDTTDGLFSAVSIS | CATCACCGCGCCGCGCGGCCCG | hIP |
| | | | PGFTGTQETMAWYGPRLASQGF | TTCGAGGTCGACGAAGAATCCG | TG2xYT |
| | | | VVFTIDTITTTDQPDSRARQLQAS | TTAGCCGCCTGAGCGTGTCCGG | |
| | | | LDYLVNDSDVKDII | TTTTGGTGGCGGCACTATCTAC | |
| | | | DPARLGVMGHSMGGGGSLKAA | TACCCCACGGATACGACCGATG | |
| | | | LDNPALKAAIPLTPWHTTKDFSG | GTCTGTTCTCCGCGGTGTCTATT | |
| | | | VQTPTLIIGAQNDTVA | TCTCCCGGGTTCACCGGCACAC | |
| | | | PVSQHAKPFYESLPDDPGKAYLE | AGGAAACTATGGCTTGGTACGG | |
| | | | LAGASHLAPNTDNTTIAKFSIAW | CCCGCGTCTGGCATCTCAGGGT | |
| | | | LKRFLDDDTRYDQF | TTCGTTGTCTTCACCATTGATAC | |
| | | | LCPPPENDDSISDYQSTCPYLEHH | CATTACCACCACCGATCAGCCA | |
| | | | HHHH | GATAGCCGTGCCCGTCAGCTGC | |
| | | | | AGGCAAGCCTGGACTATCTGGT | |
| | | | | TAACGACTCAGACGTGAAAGAT | |
| | | | | ATCATCGATCCGGCACGTCTGG | |
| | | | | GTGTGATGGGTCACTCTATGGG | |
| | | | | TGGTGGCGGCTCCCTGAAAGCA | |
| | | | | GCCCTGGATAACCCGGCGCTGA | |
| | | | | AAGCGGCAATCCCACTGACTCC | |
| | | | | GTGGCACACCACCAAAGACTTC | |
| | | | | TCCGGTGTTCAGACGCCGACCC | |
| | | | | TGATCATTGGTGCGCAGAACGA | |
| | | | | CACCGTTGCACCTGTAAGCCAG | |
| | | | | CACGCAAAACCATTTTACGAAT | |
| | | | | CTCTGCCAGATGATCCGGGTAA | |
| | | | | AGCTTACCTGGAACTGGCAGGT | |
| | | | | GCTTCCCACCTTGCTCCGAACA | |
| | | | | CCGACAACACCACTATCGCAAA | |
| | | | | ATTCTCCATCGCATGGCTGAAA | |
| | | | | CGTTTCCTGGACGATGACACTC | |
| | | | | GTTACGATCAGTTCCTGTGCCC | |
| | | | | GCCGCCGGAGAACGACGATTCT | |
| | | | | ATTTCCGACTACCAGTCTACCT | |
| | | | | GCCCGTAC | |
| Group7 | 701 | 3 | MANPYERGPNPTDALLEARSGPF | GCGAACCCGTATGAACGGGGTC | 20° |
| | | | SVSEENVSRLSASGFGGGTIYYPR | CGAACCCTACGGACGCTCTGCT | C./20 |
| | | | ENNTYGAVAISPGY | GGAAGCACGTAGCGGTCCGTTT | hIP |
| | | | TGTEASIAWLGKRIASHGFVVITI | AGTGTTTCCGAGGAGAACGTTT | TG2xYT |
| | | | DTITTLDQPDSRAEQLNAALNHM | CTCGCCTTTCTGCTTCCGGTTTT | |
| | | | INRASSTVRSRID | GGCGGCGGTACCATCTACTACC | |
| | | | SSRLAVMGHSMGGGGSLRLASQ | CGCGTGAAAACAACACGTATGG | |
| | | | RPDLKAAIPLTPWHLNKNWSSVR | TGCTGTTGCTATCAGCCCGGGT | |
| | | | VPTLIIGADLDTIAP | TATACTGGTACTGAAGCTTCCA | |
| | | | VLTHARPFYNSLPTSISKAYLELD | TTGCTTGGCTGGGTAAACGTAT | |
| | | | GATHFAPNIPNKIIGKYSVAWLK | CGCTAGCCACGGTTTTGTAGTC | |
| | | | RFVDNDTRYTQFL | ATCACCATCGATACCATCACTA | |
| | | | CPGPRDGLFGEVEEYRSTCPFLE | CCCTCGATCAGCCAGATAGCCG | |
| | | | HHHHHH | TGCGGAACAGCTGAACGCGGC | |
| | | | | ACTGAACCACATGATCAACCGT | |
| | | | | GCGTCGTCGACCGTTCGTTCTC | |
| | | | | GTATTGACTCTTCCCGCCTGGC | |
| | | | | GGTAATGGGCCACTCTATGGGT | |
| | | | | GGTGGTGGCTCGCTTCGCTTAG | |
| | | | | CCTCTCAGCGGCCGGATCTCAA | |
| | | | | GGCAGCTATTCCGCTGACCCCG | |
| | | | | TGGCACTTAAACAAAAACTGGT | |
| | | | | CTAGCGTTCGTGTACCGACCCT | |
| | | | | GATCATCGGCGCGGACCTGGAT | |
| | | | | ACTATTGCGCCGGTTCTGACCC | |
| | | | | ACGCGCGCCCGTTCTACAATTC | |
| | | | | GCTGCCGACCTCCATCTCTAAA | |
| | | | | GCATACTTGGAACTGGACGGTG | |
| | | | | CGACGCACTTCGCGCCGAACAT | |
| | | | | TCCGAACAAGATTATCGGCAAA | |
| | | | | TACTCCGTGGCTTGGCTGAAAC | |
| | | | | GTTTCGTAGACAACGATACTCG | |
| | | | | TTACACACAGTTCCTGTGTCCG | |
| | | | | GGTCCGCGTGATGGTCTGTTTG | |
| | | | | GTGAAGTTGAAGAATACCGCTC | |
| | | | | CACCTGCCCGTTT | |
| | 702 | 2 | MAANPYERGPNPTDALLEARSGP | GCTGCAAACCCGTATGAACGCG | |
| | | | FSVSEENVSRLSASGFGGGTIYYP | GTCCGAATCCGACCGACGCACT | Auto |
| | | | RESNTYGAVAISPG | GTTAGAAGCGCGATCTGGTCCA | 28° |
| | | | YTGTEASIAWLGERIASHGFVVIT | TTCTCCGTATCAGAGGAAAATG | C./23.5 |
| | | | IDTITTLDQPDSRAEQLNAALNH | TGTCCCGTCTGTCCGCGTCGGG | h |
| | | | MINRASSTVRSRI | CTTCGGCGGTGGCACCATTTAC | |
| | | | DSSRLAVMGHSMGGGGTLRLAS | TACCCGCGTGAAAGTAACACCT | |
| | | | QRPDLKAAIPLTPWHLNKNWSS | ATGGCGCTGTAGCTATCTCCCC | |
| | | | VTVPTLIIGADLDTIA | GGGCTATACTGGTACCGAAGCG | |
| | | | PVATHAKPFYNSLPSSISKAYLEL | TCTATTGCATGGCTGGGTGAAC | |
| | | | DGATHFAPNIPNKIIGKYSVAWL | GTATCGCATCCCATGGTTTTGT | |
| | | | KWFVDNDTRYTQF | AGTTATTACTATTGACACCATT | |
| | | | LCPGPRDGLFGEVEEYRSTCPFLE | ACTACGCTGGATCAACCAGACT | |
| | | | HHHHHH | CACGTGCTGAGCAGCTGAACGC | |
| | | | | AGCGCTCAATCACATGATTAAC | |
| | | | | CGCGCATCGAGCACCGTGCGTT | |
| | | | | CTCGCATCGATAGCTCTCGTCT | |
| | | | | GGCGGTGATGGGTCACTCCATG | |
| | | | | GGTGGCGGTGGCACGCTGCGTC | |
| | | | | TGGCAAGCCAGCGTCCGGATCT | |
| | | | | CAAAGCAGCGATTCCGCTGACT | |
| | | | | CCATGGCATTTGAACAAAAACT | |
| | | | | GGAGCTCTGTGACCGTGCCGAC | |
| | | | | CCTGATCATCGGCGCCGATCTG | |
| | | | | GACACCATCGCACCGGTGGCCA | |
| | | | | CTCATGCCAAACCATTCTATAA | |
| | | | | CTCCCTGCCGTCATCTATCTCCA | |
| | | | | AGGCTTACCTGGAACTGGACGG | |
| | | | | TGCGACCCACTTCGCTCCAAAC | |
| | | | | ATCCCGAACAAGATTATCGGTA | |
| | | | | AATATTCAGTAGCATGGCTGAA | |
| | | | | ATGGTTCGTTGATAACGATACC | |
| | | | | CGTTACACTCAGTTCCTGTGTCC | |
| | | | | GGGTCCGCGCGACGGTCTGTTC | |
| | | | | GGCGAAGTGGAAGAGTACCGTT | |
| | | | | CGACCTGTCCGTTT | |
| | 703 | 3 | MANPYERGPNPTDALLEARSGPF | GCCAACCCGTACGAACGCGGTC | 20° |
| | | | SVSEENVSRLSASGFGGGTIYYPR | CAAACCCGACCGACGCGCTTCT | C./20 |
| | | | ENNTYGAVAISPGY | TGAGGCCCGTAGCGGTCCATTC | hIP |
| | | | TGTEASIAWLGERIASHGFVVITI | AGCGTAAGCGAAGAAAACGTG | TG2xYT |
| | | | DTITTLDQPDSRAEQLNAALNHM | TCCCGCCTGAGCGCCTCTGGTT | |
| | | | INRASSTVRSRID | TTGGTGGTGGCACCATCTACTA | |
| | | | SSRLAVMGHSMGGGGSLRLASQ | TCCGCGCGAAAACAACACATAC | |
| | | | RPDLKAAIPLTPWHLNKNWSSVR | GGTGCGGTCGCTATCTCCCCAG | |
| | | | VPTLIIGADLDTIAP | GTTATACCGGTACCGAAGCATC | |
| | | | VLTHARPFYNSLPTSISKAYLELD | CATCGCATGGCTTGGTGAACGC | |
| | | | GATHFAPNIPNKIIGKYSVAWLK | ATTGCAAGCCATGGCTTTGTCG | |
| | | | RFVDNDTRYTQFL | TCATCACGATTGATACGATCAC | |
| | | | CPGPRDGLFGEVEEYRSTCPFLE | CACTCTGGACCAGCCGGATTCC | |
| | | | HHHHHH | CGCGCGGAACAGCTGAACGCG | |
| | | | | GCTCTCAATCACATGATCAACC | |
| | | | | GTGCGTCCTCTACCGTACGTTC | |
| | | | | GCGTATCGACAGCTCGCGCCTG | |
| | | | | GCTGTTATGGGCCATAGCATGG | |
| | | | | GTGGCGGCGGTTCGCTTCGTCT | |
| | | | | GGCTTCGCAGCGTCCGGACTTG | |
| | | | | AAGGCCGCAATCCCACTGACCC | |
| | | | | CGTGGCACCTGAATAAAAATTG | |
| | | | | GAGCTCCGTTCGTGTGCCGACC | |
| | | | | CTGATCATCGGTGCGGATCTGG | |
| | | | | ACACCATCGCGCCGGTTCTGAC | |
| | | | | TCACGCGCGCCCATTCTACAAC | |
| | | | | TCTCTGCCGACCTCTATCTCCAA | |
| | | | | AGCATACCTTGAACTGGACGGC | |
| | | | | GCGACCCACTTCGCTCCGAACA | |
| | | | | TTCCTAACAAAATCATCGGCAA | |
| | | | | GTATAGCGTAGCCTGGCTGAAA | |
| | | | | CGCTTCGTGGACAACGATACCC | |
| | | | | GCTACACCCAGTTCCTGTGCCC | |
| | | | | GGGTCCGCGCGACGGCCTGTTC | |
| | | | | GGCGAAGTAGAAGAATATCGCT | |
| | | | | CTACCTGCCCTTTC | |
| | 705 | 3 | MANPYERGPNPTDALLEARSGPF | GCTAACCCATACGAACGCGGTC | 20° |
| | | | SVSEERASRFGADGFGGGTIYYP | CGAATCCGACGGACGCCCTGCT | C./20 |
| | | | RENNTYGAVAISPGY | GGAGGCGCGTTCTGGTCCTTTC | hIP |
| | | | TGTQASVAWLGERIASHGFVVITI | AGCGTTAGCGAAGAACGTGCAT | TG2xYT |
| | | | DTNTTLDQPDSRARQLNAALDY | CCCGTTTCGGTGCTGATGGCTT | |
| | | | MINDASSAVRSRID | CGGTGGTGGGACCATCTACTAC | |
| | | | SSRLAVMGHSMGGGGTLRLASQ | CCGCGTGAAAACAACACATACG | |
| | | | RPDLKAAIPLTPWHLNKNWSSVR | GCGCGGTCGCTATCTCCCCGGG | |
| | | | VPTLIIGADLDTIAP | CTATACGGGCACACAAGCTTCT | |
| | | | VLTHARPFYNSLPTSISKAYLELD | GTGGCTTGGCTGGGTGAGCGTA | |
| | | | GATHFAPNIPNKIIGKYSVAWLK | TCGCGTCTCATGGCTTCGTTGTC | |
| | | | RFVDNDTRYTQFL | ATCACGATTGACACTAACACCA | |
| | | | CPGPRDGLFGEVEEYRSTCPFLE | CCCTGGACCAGCCGGATTCACG | |
| | | | HHHHHH | TGCCCGTCAGCTGAACGCAGCG | |
| | | | | CTCGATTACATGATTAACGATG | |
| | | | | CCTCGTCCGCTGTGCGTTCCCGT | |
| | | | | ATCGATTCTTCTCGTCTGGCAGT | |
| | | | | TATGGGTCACTCTATGGGTGGC | |
| | | | | GGCGGTACACTGCGCCTCGCCA | |
| | | | | GCCAGCGTCCGGACCTGAAGGC | |
| | | | | TGCCATCCCACTGACCCCGTGG | |
| | | | | CACCTGAACAAAAACTGGTCTT | |
| | | | | CAGTACGCGTGCCGACTCTGAT | |
| | | | | CATCGGTGCTGACCTGGACACC | |
| | | | | ATCGCGCCGGTTCTGACTCATG | |
| | | | | CGCGTCCGTTCTACAACTCTCT | |
| | | | | GCCGACCTCTATTTCGAAAGCC | |
| | | | | TATTTAGAGCTGGATGGTGCAA | |
| | | | | CCCACTTTGCACCGAACATCCC | |
| | | | | TAACAAAATTATTGGGAAGTAT | |
| | | | | TCTGTTGCATGGCTGAAACGCT | |
| | | | | TCGTGGACAACGACACCCGCTA | |
| | | | | TACTCAGTTTCTGTGTCCGGGG | |
| | | | | CCGCGCGACGGTCTTTTCGGTG | |
| | | | | AGGTTGAAGAATACCGTTCGAC | |
| | | | | TTGCCCGTTC | |
| | 706 | 3 | MANPYERGPNPTDALLEARSGPF | GCTAACCCGTACGAACGTGGCC | Auto |
| | | | SVSEERASRFGADGFGGGTIYYP | CGAACCCGACCGATGCACTCCT | 28° |
| | | | RENNTYGAVAISPGY | GGAAGCTCGCAGCGGTCCGTTC | C./23.5 |
| | | | TGTQASVAWLGKRIASHGFVVIT | TCGGTTTCGGAGGAACGTGCGA | h |
| | | | IDTNTTLDQPDSRARQLNAALDY | GCCGCTTCGGTGCAGATGGTTT | |
| | | | MINDASSAVRSRID | CGGCGGTGGCACCATCTACTAC | |
| | | | SSRLAVMGHSMGGGGSLRLASQ | CCGCGCGAAAATAACACTTATG | |
| | | | RPDLKAAIPLTPWHLNKNWSSVR | GCGCAGTGGCGATTTCGCCGGG | |
| | | | VPTLIIGADLDTIAP | TTACACCGGTACCCAGGCATCC | |
| | | | VLTHARPFYNSLPTSISKAYLELD | GTGGCATGGCTGGGTAAGAGA | |
| | | | GATHFAPNIPNKIIGKYSVAWLK | ATTGCAAGCCACGGTTTCGTAG | |
| | | | RFVDNDTRYTQFL | TTATTACTATCGATACCAACAC | |
| | | | CPGPRDGLFGEVEEYRSTCPFLE | CACTCTCGATCAGCCAGATTCT | |
| | | | HHHHHH | CGCGCGCGCCAGCTGAACGCAG | |
| | | | | CCCTCGACTACATGATCAACGA | |
| | | | | TGCGTCTTCTGCGGTGCGTAGC | |
| | | | | CGCATTGACAGCTCTCGTTTGG | |
| | | | | CAGTAATGGGCCACTCTATGGG | |
| | | | | CGGCGGTGGGTCTCTGCGTCTG | |
| | | | | GCTTCTCAGCGTCCGGACCTGA | |
| | | | | AAGCTGCAATCCCACTGACGCC | |
| | | | | GTGGCACCTGAACAAAAATTGG | |
| | | | | TCTAGCGTCCGTGTGCCGACCC | |
| | | | | TGATCATCGGTGCGGATCTGGA | |
| | | | | TACTATTGCACCGGTGCTGACC | |
| | | | | CACGCCCGCCCGTTCTATAACA | |
| | | | | GCCTGCCGACCTCCATTTCAAA | |
| | | | | AGCTTACCTGGAGCTGGATGGT | |
| | | | | GCCACCCACTTCGCTCCAAACA | |
| | | | | TCCCGAACAAAATTATCGGTAA | |
| | | | | ATATTCTGTCGCGTGGCTGAAA | |
| | | | | CGTTTCGTTGACAACGATACCC | |
| | | | | GCTATACTCAGTTCCTGTGCCC | |
| | | | | GGGTCCGCGTGATGGCCTGTTT | |
| | | | | GGTGAGGTTGAAGAATATCGCT | |
| | | | | CTACTTGTCCTTTT | |
| | 708 | 2 | MANPYERGPNPTESMLEARSGPF | GCTAACCCGTATGAGCGTGGTC | 20° |
| | | | SVSEERASRLGADGFGGGTIYYP | CGAACCCGACGGAAAGCATGCT | C./20 |
| | | | RENNTYGAIAISPGY | CGAGGCTCGTAGCGGCCCGTTT | hIP |
| | | | TGTQSSIAWLGERIASHGFVVIAI | TCTGTAAGCGAAGAACGTGCAT | TG2xYT |
| | | | DTNTTLDQPDSRARQLNAALDY | CTCGTCTGGGTGCGGATGGCTT | |
| | | | MLTDASSSVRNRID | CGGCGGCGGTACCATCTATTAT | |
| | | | ASRLAVMGHSMGGGGTLRLASQ | CCGCGTGAAAACAACACGTATG | |
| | | | RPDLKAAIPLTPWHLNKSWRDIT | GTGCTATTGCAATTTCCCCTGGT | |
| | | | VPTLIIGADLDTIAP | TATACCGGTACTCAGTCTTCCA | |
| | | | VSSHSEPFYNSIPSSTDKAYLELN | TTGCGTGGCTGGGCGAACGTAT | |
| | | | NATHFAPNITNKTIGMYSVAWLK | TGCAAGCCACGGCTTTGTGGTA | |
| | | | RFVDEDTRYTQFL | ATCGCGATCGACACCAACACCA | |
| | | | CPGPRTGLLSDVDEYRSTCPFLE | CCCTTGACCAGCCGGACTCTCG | |
| | | | HHHHHH | TGCTCGTCAGCTGAACGCTGCT | |
| | | | | TTGGATTACATGCTGACCGATG | |
| | | | | CATCTTCCTCCGTTCGTAACCGT | |
| | | | | ATCGACGCTTCTCGTCTGGCGG | |
| | | | | TAATGGGCCATTCCATGGGCGG | |
| | | | | CGGTGGCACGCTGCGTCTGGCA | |
| | | | | AGTCAGCGCCCAGACCTGAAAG | |
| | | | | CAGCGATTCCACTCACTCCGTG | |
| | | | | GCACCTGAACAAGTCCTGGCGT | |
| | | | | GATATCACCGTTCCGACCCTGA | |
| | | | | TCATCGGTGCGGACCTGGACAC | |
| | | | | CATTGCTCCGGTTTCCAGCCAT | |
| | | | | AGCGAACCATTTTATAACTCCA | |
| | | | | TCCCGAGCTCCACTGACAAAGC | |
| | | | | GTACCTTGAACTGAATAACGCC | |
| | | | | ACCCATTTCGCGCCGAACATTA | |
| | | | | CCAACAAAACGATCGGTATGTA | |
| | | | | CAGTGTGGCCTGGCTGAAACGT | |
| | | | | TTCGTTGACGAGGATACCCGCT | |
| | | | | ACACTCAGTTCCTGTGCCCGGG | |
| | | | | TCCGCGCACCGGCCTGCTGAGC | |
| | | | | GATGTTGACGAGTACCGTTCTA | |
| | | | | CTTGCCCGTTC | |
| | 709 | 0 | MANPYERGPNPTQALLEARSGPF | GCCAACCCATATGAACGTGGTC | Auto |
| | | | SVSSERAWRLGSDGFGGGTIYYP | CAAACCCTACGCAGGCGTTACT | 28° |
| | | | RENNTYGAVAISPGY | GGAGGCACGTAGTGGTCCATTC | C./26 |
| | | | TGTQASVAWLGERIASHGFVVITI | AGCGTTTCCAGCGAACGTGCTT | h |
| | | | DTNTTLDQPDSRARQLDAALDH | GGCGCCTGGGCAGCGACGGTTT | |
| | | | MLNDASSAVRSRID | CGGCGGTGGCACGATTTACTAC | |
| | | | RNRLAVMGHSMGGGGTLRLASQ | CCGCGCGAAAACAACACCTACG | |
| | | | RPDLKAAIPLTPWHLNKSWSNV | GTGCGGTGGCCATCAGCCCGGG | |
| | | | QVPTLIIGADLDTIAP | CTATACCGGTACCCAGGCTTCT | |
| | | | VLTHAEPFYNSIPTSTRKAYLELD | GTAGCGTGGCTGGGTGAACGTA | |
| | | | GATHFAPNITNSTIGMYSVAWLK | TTGCGTCCCACGGCTTCGTGGT | |
| | | | RFVDEDTRYTQFL | GATCACGATCGATACCAATACT | |
| | | | CPGPRTGLFSDVEEYRSTCPFLEH | ACCCTGGATCAGCCGGACTCTC | |
| | | | HHHHH | GTGCTCGCCAGCTGGACGCTGC | |
| | | | | ATTAGATCACATGCTGAACGAC | |
| | | | | GCTAGTTCCGCGGTCCGCTCTC | |
| | | | | GTATCGACCGTAACCGTTTGGC | |
| | | | | GGTAATGGGTCACTCTATGGGT | |
| | | | | GGTGGCGGTACCCTTCGCCTGG | |
| | | | | CGAGCCAGCGCCCAGACCTCAA | |
| | | | | GGCTGCAATCCCTCTGACGCCG | |
| | | | | TGGCACCTGAATAAGAGCTGGT | |
| | | | | CTAATGTCCAGGTTCCAACTCT | |
| | | | | CATTATTGGGGCGGACCTCGAC | |
| | | | | ACGATCGCGCCGGTACTGACCC | |
| | | | | ACGCAGAACCGTTCTATAACTC | |
| | | | | AATCCCGACCAGCACCCGTAAA | |
| | | | | GCATATCTTGAACTCGATGGTG | |
| | | | | CCACCCACTTTGCACCGAACAT | |
| | | | | CACCAACTCTACCATCGGCATG | |
| | | | | TATTCCGTTGCGTGGCTTAAAC | |
| | | | | GTTTTGTGGATGAAGACACCCG | |
| | | | | TTACACCCAATTCCTGTGCCCG | |
| | | | | GGCCCACGCACCGGTCTCTTTT | |
| | | | | CTGACGTAGAAGAATACCGTTC | |
| | | | | TACCTGCCCGTTC | |
| | 711 | 2 | MANPYERGPDPTQASLEASRGPF | GCGAACCCGTACGAGCGTGGTC | 20° |
| | | | PVSEERVSSPVSGFGGGTIYYPQE | CGGACCCGACTCAGGCGTCCCT | C./20 |
| | | | NNTYGAVAISPGYT | GGAAGCCTCTCGTGGCCCGTTC | hIP |
| | | | ATQSSVAWLGERIASHGFVVITID | CCGGTTTCTGAAGAGCGTGTTT | TG2xYT |
| | | | TNTTLDQPDSRADQLEAALDHM | CTTCTCCAGTAAGCGGCTTCGG | |
| | | | VDGASSTVRSRIDR | GGGCGGCACAATTTATTACCCG | |
| | | | NRLAVMGHSMGGGGTLRLASRR | CAGGAAAACAACACCTACGGC | |
| | | | PDLKAAIPLTPWHLNKSWSNVQ | GCGGTGGCAATCTCTCCGGGCT | |
| | | | VPTLIIGAENDTVAPV | ATACTGCTACCCAGTCCTCTGT | |
| | | | ALHAEPSYTSIPTSTRKAYLELNG | GGCTTGGCTGGGAGAACGCATT | |
| | | | ASHFAPSVANATIGMYGVAWLK | GCATCACACGGCTTTGTTGTTA | |
| | | | RFVDEDTRYTRFLC | TCACGATCGACACCAACACCAC | |
| | | | PGPRTGLFSDVEEYRSTCPFLEHH | TCTGGACCAGCCGGATTCGCGT | |
| | | | HHHH | GCAGACCAACTGGAAGCTGCGC | |
| | | | | TGGATCACATGGTAGATGGCGC | |
| | | | | GTCCTCTACCGTTCGCTCTCGCA | |
| | | | | TCGACCGTAACCGCCTGGCAGT | |
| | | | | AATGGGTCATAGCATGGGTGGC | |
| | | | | GGCGGTACTCTGCGCCTGGCAT | |
| | | | | CTCGTCGCCCGGATCTGAAAGC | |
| | | | | GGCGATCCCGCTGACCCCATGG | |
| | | | | CACCTGAACAAAAGCTGGTCCA | |
| | | | | ACGTTCAGGTCCCTACCCTGAT | |
| | | | | CATTGGCGCCGAGAATGACACG | |
| | | | | GTTGCCCCGGTAGCACTGCACG | |
| | | | | CGGAACCGTCCTACACCTCCAT | |
| | | | | CCCAACCTCCACCCGTAAAGCT | |
| | | | | TATCTGGAACTGAACGGTGCGT | |
| | | | | CTCACTTTGCGCCGAGTGTCGC | |
| | | | | TAACGCTACTATTGGCATGTAC | |
| | | | | GGTGTTGCGTGGCTGAAACGCT | |
| | | | | TTGTCGATGAAGACACACGTTA | |
| | | | | CACCCGTTTCCTGTGTCCTGGTC | |
| | | | | CGCGTACCGGCCTGTTCTCCGA | |
| | | | | TGTGGAAGAATACCGTAGCACT | |
| | | | | TGCCCATTC | |
| | 712 | 2 | MANPYERGPNPTNSSIEALRGPY | GCGAATCCGTACGAACGTGGTC | 20° |
| | | | SVSEDSVSSLVSGFGGGTIYYPTG | CTAACCCAACCAACTCAAGCAT | C./20 |
| | | | TNETFGAVAISPGY | CGAGGCTCTGCGCGGGCCATAC | hIP |
| | | | TGTQSSISWLGPRLASQGFVVMT | AGCGTGTCAGAGGACTCGGTTT | TG2xYT |
| | | | IDTNTTLDQPDSRASQLDAALDY | CGAGCTTGGTGAGCGGTTTCGG | |
| | | | MVNRSSSTVRNRIDLEHHHHHH | GGGCGGCACCATCTACTACCCG | |
| | | | | ACCGGTACCAATGAAACTTTTG | |
| | | | | GCGCGGTGGCAATCAGCCCGGG | |
| | | | | TTACACGGGTACGCAGTCTTCT | |
| | | | | ATTTCTTGGCTGGGCCCTCGTCT | |
| | | | | GGCGTCCCAGGGTTTCGTTGTT | |
| | | | | ATGACCATTGATACTAACACTA | |
| | | | | CCCTGGATCAGCCGGACTCTCG | |
| | | | | CGCCTCTCAGCTGGATGCAGCA | |
| | | | | CTGGACTATATGGTGAACCGTT | |
| | | | | CTTCATCTACCGTGCGCAATCG | |
| | | | | TATCGAC | |
| | 714 | 3 | MANPYERGPNPTDALLEARSGPF | GCGAACCCTTACGAACGCGGTC | Auto |
| | | | SVSEENVSRLSASGFGGGTIYYPR | CGAACCCGACCGATGCCCTGCT | 28° |
| | | | ENNTYGAVAISPGY | CGAAGCTCGCTCGGGCCCGTTC | C./23.5 |
| | | | TGTEASIAWLGERIASHGFVVITI | TCTGTCTCCGAAGAAAACGTGA | h |
| | | | DTITTLDQPDSRAEQLNAALNHM | GCCGTCTGTCGGCTTCCGGCTTT | |
| | | | INRASSTVRSRID | GGCGGTGGCACAATTTACTATC | |
| | | | SSRLAVMGHSMGGGGSLRLASQ | CTCGCGAGAACAACACCTACGG | |
| | | | RPDLKAAIPLTPWHLNKNWSSVT | TGCTGTTGCGATCTCTCCGGGC | |
| | | | VPTLIIGADLDTIAP | TATACTGGTACAGAGGCTTCCA | |
| | | | VATHAKPFYNSLPSSISKAYLELD | TCGCCTGGCTGGGCGAGCGCAT | |
| | | | GATHFAPNIPNKIIGKYSVAWLK | CGCTTCTCACGGTTTCGTTGTCA | |
| | | | RFVDNDTRYTQFL | TTACCATCGATACTATTACCAC | |
| | | | CPGPRDGLFGEVEEYRSTCPFYL | CCTGGACCAGCCGGACTCGCGT | |
| | | | EHHHHHH | GCTGAACAGCTTAATGCAGCGC | |
| | | | | TTAACCATATGATCAATCGTGC | |
| | | | | TTCGTCAACCGTTCGCAGCCGT | |
| | | | | ATCGATTCTTCTCGTCTGGCGGT | |
| | | | | GATGGGTCATTCTATGGGTGGC | |
| | | | | GGTGGTTCGCTCCGTCTGGCCA | |
| | | | | GCCAGCGCCCGGATCTGAAAGC | |
| | | | | GGCAATCCCGCTGACTCCGTGG | |
| | | | | CATCTGAACAAAAACTGGTCTT | |
| | | | | CGGTTACCGTGCCGACCCTGAT | |
| | | | | TATCGGTGCAGACCTGGACACG | |
| | | | | ATTGCACCGGTTGCGACTCACG | |
| | | | | CAAAACCGTTCTACAACTCCCT | |
| | | | | GCCGTCTTCTATTTCTAAGGCAT | |
| | | | | ACCTTGAACTGGACGGTGCAAC | |
| | | | | CCATTTCGCTCCGAACATTCCG | |
| | | | | AACAAAATCATCGGTAAATACA | |
| | | | | GCGTGGCCTGGCTGAAACGTTT | |
| | | | | TGTTGACAACGACACCCGTTAC | |
| | | | | ACACAGTTCCTGTGCCCGGGTC | |
| | | | | CGCGTGACGGTCTTTTCGGCGA | |
| | | | | GGTGGAAGAATATCGTAGCACC | |
| | | | | TGTCCATTCTAC | |

In an embodiment, the sequences disclosed herein are as follows:

>PETcan_101
CLYLNIWTPDLNGSLPVMVFIHGGGNQQGSTAQIAGGARIYEGKNLARRGQVVVVTLQYR
LGALGYLVHPGLEAESTHGKAGNYGALDQLAALLWIKENIRAFGGDPELVTLFGESAGAV
NIGNLLVMPAAKGLFHRAILQSGSPRLKAYSAARNEGIAFAQKLGAAGTPEQQVAHLRTL
PVDSLVKGDSNPISGGSMAQGSWQPVLDGYWFPQAPLDAMRSGEHHRVPLIVGSSSDEMS
LYVPSVVTPLMLQTFVQTTIPAPYRQQVLALYPPGTTNEQARASYVALVGDPLESTCRHA
S
>PETcan_102
QSPAQSSAPTVELDSGAIAGSTADGVVSFKGIPYAAPPVGNLRWRAPQPVASWTGVRAAT
EYGYDCIQLPLEGDAAASGGEMSEDCLVLNVWRPAEIAPGERLPVLVWIHGGGFLNGSAA
APIYDGTAFAQQGLVVVSFNYRLGRLGFFAHPALTAANEGPLGNYGLMDQIAALEWVQRN
IAAFGGDPARITLMGQSAGGISVMYHLTAPESQGLFHQAAVLSGGGRTYLLGLRNLREST
DALPSAEQSGLAFGRRFGIRGRGRAALRSLRSLSAEEVNGDLSMAALVEKPADYAG
>PETcan_103
QGITVRTPLGPALGQMEKGAIAFYGLPYAQASRFEAPRPVAAWPPGVGRERVACPQTPGT
TARLGGYIPPQREDCLVANLFLPLEPPPPEGFPVMVYLHGGGFTSGSAAEPIYGGHRMAQ
EGVVVVSVNYRLGPLGFLALPALEKENPKAVGNYGLLDLVEALRFVQRHIRYFGGNPQNV
TLFGESAGGMLVCTLLATPEAQGLFHKAILQSGGCHQVRPLERDFPFGEQWAKNLGCSPE
DLACLRNLPLSRLFPTMEPKAPPDITASALGFPNSPFKPHLGALLPESPTEALRKGQARD
IPLLVGANLEELAFPGLAWLLGPRRWEEFGQRLAAQGLTQQQREALKGVYQKRFSEPRAA
WGQAQTDLLLLCPSLKAARLQASFAPTYAYLFTFRVPGFEGLGAFHGLELAPLFGNFEEM
PFLPLFLSAEAREKAEALGKRMRRYWVSFAREGEPRSWPHWPTYEEGYLLRLDEPPGLIP
DLYEERCGVLEALGLL
>PETcan_104
VFLGWQGSPVQLPAHAGEQAPSPVEPLNLPDPARPGAYPVALLTYGSGQDKLRQEYAQGA
ALLTPSVDASLLLEGWSSLRTAYWGFSPAELPLNGRVWYPQAEGRFPLVIAVHGNHPMEE
TSESGYDYLGELLASRGFIFVAVDENFLNISAWGDVLFFNRLEGESDARGWLVLEHLRLW
QSWNEQPGNPFYQRVDLNQIALLGHSRGGEAIVIAAAFNRLSHYPDNAALSFDYGFKIRS
LIALAPADGQYQPGGLPTPLQDVNYLLLHGSHDMDVLTMMGAAPFERLTFSGQDDFFKSA
VYIYGANHGQFNSVWGNKDIAEPIPRLYNLRQLLPQTEQQRIAQVLISAFLEDTLRGERA
YRPLFQ
>PETcan_201
LVRIGEQEDAVAALEFLLQRDEIDTERIALAGYSFGAFVGLAALNGNENIKALVGVSPPL
TLFEFSYLKNCTKPKLLIIGDMDQFTPLKVFKEFYEKIPEPKNKRIIEGADHFYWGYENE
VGQVVADFLKKTFKNIP
>PETcan_202
VDITGNGMAATAPTDERIVDKPLPQPQIRSGNVRAMPAARKLAQEHGIDLSTLTGSGPGG
VIVKEDVERAITARAVPVSPLQRVNFYSAGYRLDGLLYTPRHLPAGERRPGVVLLVGYTY
LKTMVMPDIAKVLNAAGYVALVFDYRGFGESEGPRGRLIPLEQVADARAALTFLAEQSMV
DPDRLAVIGISLGGAHAITTAALDQRVRAVVALEPPGHGARWLRSLRRHWEWRQFLSRLA
EDRRQRVLSGGSTMVDPLEIVLPDPESQAFLDQVAAEFPQMKVTLPLESAEALIEYVSED
LAGRIAPRPLLIIHSDADQLVPVAEAQAIAERAGSSAQLEIIPGMSHFNWVMPGSPGFTR
VTDSIVKFLRNTLPVSADN
>PETcan_203
VPLILNVHGGPAGVFQQTFTGGRSIYPIATFAARGYAVLRPNPRGSSGYGVEFRRANLKD
WGGMDYQDLMAGVDKVIEMGVADSSRLGVMGWSYGGFMTSWIVTQTNRFKAASAGAPVTN
LTSFTTTADIPAFIPDYFGGQFWDSPEVYRAHSPISFVKSVTTPTMIQHGTADMRVPISQ
GFEFYNALKARGIPTRM
>PETcan_204
VPSAGVGLSGVLHLPAGVSRPVLFLHGFTGNKTESGRLYTDMARVLCSAGYAALREDFRG
HGDSPLPFEEFRISLAVEDARNAAGFLKNVPEVDGTRFGVVGLSMGGGVAVSLAAGREDV
GALVLLSPALDWPELFQRARGFFRAEEGYVYWGPHRMRDVYAMETMNFSVMGLAEEIQAP
TLIIHSVDDMVVPISQAKRFYEKLKVEKKFIEIEHGGHVFDDYNVRRRIEQEVLDWVKRH
L
>PETcan_205
RVLCSAGYAVLRFDYRCHGDSPLPFEEFRISMAVEDAENAVKYVKSLERVDGSSFAVIGL
SMGGGVAVKLAAGRDDVAALVLLSPALDWPELTGRVPFKVEEGYVYMGPFRMRAENAMEN
ARFTVMDIAEQVKAPTLIVHATDDEVVPISQAKRFYEKLRVEKRFLEVKSGHVFNDYHVR
RNLEGEILSWVKSHL
>PETcan_206
VPSAGVGLSGVLHLPAGVSRPVLFLHGFTGNKTESGRLYTDMARVLCSAGYAALRFDFRC
HGDSPLPFEEFRISLAVEDARNAAGFLKNVPEVDGTKFGVVGLSMGGGVAVSLAAGREDV
GALVLLSPALDWPELFQRARGFFRAEEGYVYWGPNRMRDVYAMETMNFSVMGLAEEIKAP
TLIIHSVDDVVVPISQAKRFYEKLKVEKKFIEIEQGGHVFEDYNVRRRIEREVLDWVKRH
L
>PETcan_207
GFTGNKAEAGRLYTDMARVLCAAGYAALRFDFRCHGDSPLPFEEFRISYAVEDARNAASF
LKIQPSVDGSRFAVIGLSMGGGVAVSLAAGRDDVAALVLLSPALDWPELAARIPQPKVEG
GYVYMGPNRMKVECVTETMKFTVMDLAERVKAPTLIIHAADDMVVPISQSKRFYEKLKVE
KKFMEIERSGHVFDDYNVRRRVEAEVLDWIKKHL
>PETcan_208
DGCIEDLRFIEFDGFRLASTIHRPAIATSSAVLMLHGFTGNRIEVNRLYVDIARRLCSEG
MVVLRLDYRGHGESSLPFEEFKIGYALEDGGKALEVLQKLFNPVRIGVVGFSLGGYVAIH
LASRYRGAISSLALLAPGIKMDELATELARKLSLEGDFYIVRALKIRREGIESMIRSPSA
MIYADTVDIPVLIIHAKNDSAVPYIHSIEFYEKIRSQKKRIVILDEGGHTFELHHIRDRV
IEEVVAWFRETLLYT
>PETcan_209
VDITGNGMAATAPTDERIVDKPLPQPQIRSGNVRAMPAARKLAQEHGIDLSTLTGSGPGG
VIVKEDVERAITARAVPVSPLQRVNFYSAGYRLDGLLYTPRHLPAGERRPGVVLLVGYTY
LKTMVMPDIAKVLNAAGYVALVFDYRGFGESEGPRGRLIPLEQVADARAALTFLAEQSMV
DPDRLAVIGISLGGAHAITTAALDQRVRAVVALEPPGHGARWLRSLRRHWEWRQFLSRLA
EDRRQRVLSGGSTMVDPLEIVLPDPESQAFLDQVAAEFPQMKVTLPLESAEALIEYVSED
LAGRIAPRPLLIIHGDADQLVPVAEAQAIAERAGSSAQLEIIPG
>PETcan_210
LIRPVAFRNMNQQIIGILHTPDNIKPGEKTPGILMLHGFTGNKTEAHRLFVHVARSLSEY
GFIVLRFDFRGSGDSDGEFEDMTLPGEVSDAERALTFLLRRRNIDRDRVGVIGLSMGGRV
AAILASKDKRVKFAVLYSPALGPLRDRSLSFMSREKIERLNSGEAVEFFAEGWYIKKTFF
ETVDYIVPLDIMDSIRVPVLIVHGDRDPIIPVEEAIRAYEKIKGVNKKNELYIVRGGDHT
FSKKEHTQEVIKKTLDWIRALSVSEGSIVLFRLLE
>PETcan_211
LIRPVTFRNMNQQIIGILHTPDNIRLNEKVPGILMFHGFTGNKTEAHRLFVHVARSLSEH
GFIVLRFDFRGSGDSDGEFEDMTLPGEVSDAERALTFLLRQRNVDKNRIGVIGLSMGGRV
AAILASKDRRVKFAVLYSPALGPLRDRSLSFMSKEKIERLNSGEAVEFFAEGWYIKKAFF
ETVDYIVPLDIMDSIKVPVLIVHGDKDPLIPVGEAIRAYEKIKGVNEKNELYIVRGGDHT
FSKKEHTLEVIKKTLDWIRSLGI
>PETcan_212
LTITAIIYLLATIIAAILLVVYIISSSASKKLATPPRKTGSWSPRDLGFEYEKVEVKTSD
GLTLRGWLIPRGSEKTVIVIHGYTSCKWDEWYMKPVINILARHDFNVVAFDMRAHGESDG
EKTTLGYREVDDIGAIINYLKERGLASRLGIIGYSMGGAITLMSLARYEELKAGVADSPY
IDIRASGKRWINRVGAPLRYILLASYPLIMRLTASRTGASPEKLVMYQYAKSITKPLLII
GGQQDDLVAIDEVRKFYEEVKKVNSNVELWETTSKHVSAIQDYPREYEERIVGFFNRWL
>PETcan_213
SELELNEVFKLIKLVSFMNKGQQIIGVLHKPDKIKPHEKVPGIVMFHGFTGNKSEAHRLF
VHIARGLSSRGFMVLRFDFRGSGDSDGDFEDMTLPEEVSDAERAITFVLRQRNVDREKIG
VIGLSIGGRVAAILASRDERIKFAVLYSPALGRLKERFLSLMGEEALRRLNCGEPIEVSS
GWYLKKAFFETVDYIVPVEVMSNIRVPVLIIHGDRDEIIPVEESMKAYERIKGLNEKNEL
YIVKGGDHTFSKREHTLEVLNKTIEWLSSLNLM
>PETcan_214
ARAAPISPLQRVNFYSAGYRLDGLLYTPRHLPAGERRPGVVLLVGYTYLKTMVMPDIAKV
LNAAGYVALVFDYRGFGESEGPRGRLIPLEQVADARAALTFLAEQSMVDPDRLAVIGISL
GGAHAITTAALDQRVRAVVAIEPPGHGAHWLRSLRRHWEWSQFLSRLTEDRRQRVLSGVS
STVDPLEIVLPDPESQAFLDQVAAEFPQMKVTLPLESAEALIEYVPEDLAGRIAPRPLL
>PETcan_215
ATVLVIPKLGLTMTEGRVGRWLKQLGEPVQAGEPVLEVETEKLTVEVEAPASGILAYILA
EEGVVLPVTAPVAVIAEPGEAVDLASLLPATSGAAATPVMAASSTMQEQARAQGPTPTGE
IRATPAARKLARDHGIDLARVRGTGPGGRITAEDVERYLASQGTAWPRGEPVRFWSDGLA
LAGELFLPPSTDTAVPGVVLCTGIQGLKELGMPLLAQALADAGYAALIFDYRGFGASEGP
RGRLLPQERIRDARAALTFLETHPLIDRTRLAILGLSLGGAHALSLAAIDDRVQACIAIA
PLTNGRRWLRSLRAEWQWRV
>PETcan_301
QPYPVGTRTITYQDPVRNNRNIQTYLYYPATAAGANQPVAGGQFPVVVVGHGFTMNYAPY
AFWGNALAESGYIVAIPNTETGFSPSHSAFAADMAFLVAKLYTENTNSSSPFYQHVQYNS
CIIGHSMGGGCTYLAAQNNADVSATVTFAAAETNPSATAAAANVNCPSLVFSGSADCITP
PAQHQVPMYNALPDCKAYGGSSRVDLQACK
>PETcan_302
VRRPNNTTFTAQLYYPATATGDNAPYDGSGAPYPAVSFGHGFLQPPERYRSILEHLASWG
YLTIATESGQELFPNHRAYAEDMRYCLTYLEEQNADPASWLFGQVATAQFGISGHSMGGG
ASILAAAADARIKAVANLAAAETNPSAIQASPNITVPHSLISGSADTITPLSSNGLRMYT
AGLRAEAAARHSRRLGLRVPKTPSIFGCDSGSLPPRHA
>PETcan_303
IWYPAVRVRGQPQRTTYQYGPLIGEGRAYRDAPADLRGAPYPLLIFSHGLGGARIQSVFY
AEHLASHGFVVMAADHTGSTFADLLRGRADSILESFARRPLEILRQIEYAAALNADDDTL
RGAIDAETVGVTGHSFGGYTALAAAGAQLNINAIREGCESGKLPEQQCLFVRSEEIIWRA
RGLSAAPEGLYPPTTDPRIKAVVALAPSSAPTFGEAGAAALRVPLMIIVGSKDQATPPER
DSYPIYQSVSSAQKALVVFENAGHYIFVEQCVPALIALGRFEQCSDLVWDMQRAHDLINH
FATAFFLHALKGDPAAKAALDPTAVQFIGITYRRDGAW
>PETcan_304
IVLLLNFDVEYKRIKFNGDYIDIYKPKAEGNYPFVIFSGGMNSPSSRYESFGKFLASNGF
ITIIPDYKGWLFLLLIPLKILRIIDNLNKIDSSIKNEGCLGGHSLGAYFSMIVSYKRSSV
KCLFLFSPPALFLNYSKIKVPVLIFAGTNDEITKFEANQKIIYEHLKTQKKLVLIEGGNH
NGYMDRWDFVEALTDGYLGIEHKKQLEIVRDSVLKFLKEILLK
>PETcan_305
QVIQQTVTLQKTQLRLTKEGFVTNYRFPVDFYYPDSPESFPVILISHGFGSVRENFRTLA
QHLASHGFLVAVPQHIGSDLQYRQELIKGTLSSALSPVEFLARPTDLSTIIDYLQATQNT
GSWQKRANLQQIGVIGDSLGGTTALTIGGAPLDIPRLQTKCTSDNVIVNVALILQCQASF
LPPSEYNLADSRVKAVIATHPLISGIFSPDSLAKIQIPVMITAGNFDIITP
>PETcan_306
KVKSKPLTLYNVSGDRITADVHFVESFLPAPVVIYSHGFLGFKDWGFIPYVAERFAENGF
VFVRFNFSHNGIGENPNKITEFDKLAKNTISKQIEDLTAVIEYVFSDEFGVLNDGQLFLL
GHSGGGGISIIKAVEDERVRALALWASISTFRRYSKHQIEELEKNGYIFVRVPDSVIQVK
IEKIVYDDFVENSERYDIIKAISKLKIPILIVHGTADAIVPLAEAEKLRNSNPEYTKLVL
ISGANHLFNVKHPMEHSTDQLDKAIDETVLFFKKIIENKKAD
>PETcan_307
QTVTSMLKDLDAVITQVSEKFPQIDNKRVCLIGHSQGAYVSFLHATKDERIKCLVSWMGR
LSDLKEFWSKLWFDEIERKGYIYEWDYKITKKYVRDSLKYNLSKAAWRIKVPTLLIYGEL
DDIVPPSEGMKFYRNIKSPKKIVIVKDLNHTFSGEKAKKSVIRITLKWLSKWLKRLD
>PETcan_308
LKIIEDFASLDTGVKVFYRCILPESFKELAIVSHGFTSHSGFYIHIGKELASYGYGVCIH
DQRGHGRTAQNLERGYVDSFNDFLVDLETFTMHVQRVFGGERTVLIGHSMGGLIVLLYAG
KYGRVGDAVVAVAPAVLIPETRRFSTLIFATIASILFPRKRIELPFTEQQIEEGMKRMDR
ELLEAMGKDELVLRDTTIKLLVEIWKASREFWRYVERIQIPTLLIHGEKDNIIPIEASRR
TYSRLKTLKKELIVYPECGHSPLHEIGWRERIKNMVEWIRNNI
>PETcan_401
ANPPGGDPDPGCQTDCNYQRGPDPTDAYLEAASGPYTVSTIRVSSLVPGFGGGTIHYPTN
AGGGKMAGIVVIPGYLSFESSIEWWGPRLASHGFVVMTIDTNTIYDQPSQRRDQIEAALQ
YLVNQSNSSSSPISGMVDSSRLAAVGWSMGGGGTLQLAADGGIKAAIALAPWNSSINDEN
RIQVPTLIFACQLDAIAPVALHASPFYNRIPNTTPKAFFEMTGGDHWCANGGNIYSALLG
KYGVSWMKLHLDQDTRYAPFLCGPNHAAQTLISEYRGNCPY
>PETcan_402
AFAITPSPTPTPDPTPNPSPDPGSCSGAECYIRGPNPTVRALEADDGPYSVRTTNVSSFV
SGFGGGTIHYPVGTEGKMGAIAVIPGYVSYESSIRWWGSRLASWGFVVITIDTNTIYDQP
DSRANQLSAALDYVIAQSNSRNSSISGMVDSNRLGVIGWSMGGGGSLKLSTQRTLKAAIP
QAPWYSGFNSFNRITTPTLIIACELDVVAPVGQHASPFYNRIPSSTAKAFLEINGGDHFC
ANSGYPNEDILGKYGVSWMKRFIDGDRRYDQFLCGPNHESDRSISDYRETCNY
>PETcan_403
TTPTPTPEPEPEPPGGCGDCYQRGPDPTVAALEADRGPYSVRTINVSSWVSGFGGGTIHY
PVGTQGTMGAIAVIPGYVSYENSIEWWGGRLASWGFVVITIDTNSIYDQPDSRANQLSAA
LDYVIAQSNSSRSAIQGMVDPNRLGAIGWSMGGGGTLKLSTDRYLKAAIPQAPWYSGFNP
FDEITTPTLIIACQLDAVAPVAQHASPFYNEIPNSTAKAFLEIRNGDHFCANSGYPDEDI
LGKYGVAWMKRFIDDDRRYDAFLCGPNHEAEWDISEYRDTCNY
>PETcan_404
ADNPYQRGPDPTERSVTARRGPFAIDEISVNGGIGAGFNRGTIFYPTDRSQGTFGAVAVI
PGFLSPESLVRWFGPRLASQGFVVMTLTTNGLTDTPESRSEQLLAALDYLTTRSQVRDRI
DPSRLAVMGHSMGGGGSLAAAAKRPTLRAAIPLAPWSLTKNWSDLTVPTLIIGAENDNVA
PVAGHSERFYDSMTNVPEKAYLEMAGGNHVDPTAESDLVAKFTISWLKRFVDDDTRYDQF
LCPAPRPNRQISEYRDTCPHS
>PETcan_405
QADTDTTAVAPAAANPYERGPAPTEASVTAARGPFAIAQVNVPSGSGAGFNDGTIYYPTD
TSQGTFGAVAVIPGFISPQAVIQWFGPRLASQGFVVFTLDSNGLADLPDARGRQLLAALD
YLTTQSTVRTRIDPNRLAVMGHSMGGGGTLLAAENRPTLKAAIPLAPWEPDTSWEGVKVP
TMIIGGESDVVAPVSSMAIPDYNSLSSAPEKAYLELRSGDHLAPASESPTVAEYALSWLK
RFVDDDTRYDQFLCPGPTPDTDISQYLDTCPNGS
>PETcan_406
RFRVAASLPAEYLAVDNVVLEGTAQPPAPGGSGYQKGPEPTAALLEAGTGPFATASVTLS
RSAASGFGGGTIHYPQGVAGPFAAVAVVPGYLAAESTIAWWGPRLASHGFVVITMATNNT
LDLPASRSAQLTAALNQLKTLSATPGHAVFGLVDPNRLGVVGWSYGGGGTLLNAQANPQL
KAAMALAPKTLLQGDFTGTTVPTLVVGCQADTTAAPAFWAIPFYNKVSASTGKAYLEVRG
GSHFCVTSSTSDADKKALGKYGVAWLKRFMDEDTRYAPFLCGAPRQADVAGNAAISDYRD
NCPY
>PETcan_407
ADNPYQRGPDPTRDSVAASRGTFATASTTVGSGNGFGAGFIYYPTDTSQGTFGAVAIVPG
YTATWAAEGAWMGHWLASFGFVVIGIDTINRNDWDTARGTQLLAALDYLTQRSTVRDRVD
ASRLAVMGHSMGGGGAMYAALQRPSLKAAVGLAPFSPSQNLNGMRVPTMLLAGQHDTTTT
PASITSLYNGIPAATEKAYLELSGAGHGFPTSNNSVMMRKVIPWLKIFVDSDVRYTQFLC
PLMDNTGIRSYQSTCPLLPGTPTPPNRYEAETSPAVCTGTIASNHTGYSGTGFCDGNNAT
NAYAQFTVNASAAGSMTLRVRFANGTTTARPASLIVNGSTVQTPSFEGTGAWTTWATKTL
TVTLNAGNNTIRFNPTTANGLPNLDYIEIAAP
>PETcan_408
KPITFTLLFIFICSIFYSQCEEVNLESISNSGPYAVGSLIEGVDPIRNGPDYDGATIYYP
INGTPPYSGIAIIPGYCGVESDIQDWGPFYASHGIVAITLGTNDPCADWPSARSTALLDA
IVTVKEENSRQDSPLKDKIDVNSFAVSGWSMGGGGSQLAASIDPSLKAVIGLCPWLDLNG
FEPSDLIHDVPVLIFTGENDDIANSAEYGYMHYQGTPSTTDKLYFEIANGGHGAANSPEL
EGGEVGVYALSWLKTYLDNDPCYCEFLVNTPSNSSDYETNIECLNAGIDEGENLIHFIYP
NPIQDYIEFSNDGMERTYELKSSNGKSIKSGIVSHGYNKILFEKQNTEIYFLIIAGKSYK
LISIK
>PETcan_409
GDCPATAICRSESPGAYSGNGPYGSRSYTLSRFQTPGGATVYYPANAEPPYAGMVFTPPY
TGTQAMFAAWGPFFASHGFVLVTMDTSTTLDSVDQRAAQQKEVLNALKSENTRSGSPLRG
KLDTARLGAVGWSMGGGATWINSAEYSGLKTAMSLAGHNLTAVDIDSKGYNTRVPTLLFN
GAQDLTYLGGLGQSDGVYNNIPAGIPKVFYEVSSAGHFDWGSPTAANRSVASLALAFHKA
YLDGDTRWLQYITRPSSDVTTWRTANIR
>PETcan_410
SQVPPTDPQDAPLGECPATALCRSEAPGSYSGNGPYGYRSYSLSRLQTPGGATVYYPANA
EPPYSGLVFTPPYTGVQFMYAAWGPFFASHGIVLVTMDTTTTLDTVDQRARQQKTVLDVL
KGENNRAASPLRGKLDTSRIGAVGWSMGGGATWINAAEYAGLKTAMSLAGHNLSAIDPNA
RGYNTRVPTLLFNGALDATYLGGLGQSDGVYNAIPAGIPKVFYEVASAGHFDWGSPTAAN
RDVAGIALAFHKAFLDGDTRWVDYIRRPSRDVATWRTAYLPD
>PETcan_411
ADCPAGAICRYDEQPGGYTGDGPYRVGDYSISTFQAAGGATVYYPTNATPPFAALVFCPP
YTGVQYMYRDWGPFFASHGIVMVTMDSETTLDTVDQRADQQREVLDFLKRENTNSRSPLY
GKLATDRFGVTGWSMGGGATWINSADYSGLKTAMSLAGHNLTALDPDSRGYSTRIPTLIM
NGALDTTYLGGLGQSDGVYNAIPYGVPKVFYEVSSAGHFAWGSPTSASDDVAKVALAFQK
TFLEGDTRWAEYIRRPFWGASEWETANLP
>PETcan_412
SQVPPTPPTDDPMGDCPSTAICRGEAPGSYSGNGPYGSRSYTLSRFQTPGGATVYYPSNA
EPPYSGLVFTPPYTGTQAMFRAWGPFFASHGIVLVTMDTSTTVDTVDQRASQQKRVLDVL
KQENTRSGSPLRGKLDTSRLGAVGWSMGGGATWINSAEYNGLKTAMSLAGHNMTAIDLDS
KGGNTRVPTLLFNGALDLTMLGGLGQSIGVYNAIPRGIPKVIYEVASAGHFDWGSPTAAN
RSVAGIALAFHKTFLDGDTRWVSYIKRPSSDVATWRTENLPQ
>PETcan_413
NKEKSSFDQTAKITTRSKSIFKTIFTYLLVLAFITTIFPMNAFANSPAIIRNEEAPGKYA
GNGPFSYNSYRLPLLSVYGTGGATVYYPTSGTAPYSGLVYCPPYTAKQSALAAWGPFFAS
HGIILVTFDTLTPLDPVSLRALQQRTVLNALKTENSRLNSPLYQKVATDRIGAMGWSMGG
GATWINSAEYSGLKTAMTIAGHNLSSTNLNSKGYNTKCPTLIMNGAMDTTGLGGLGQSNG
VYKNIPANVPKVLYEVASAGHLNWTSPISASNDVAAIALAFQKTYLDGDSRWLAFITRPN
SNVSIWETSNLMNP
>PETcan_501
SNPYQRGPNPTRSALTADGPFSVATYTVSRLSVSGFGGGVIYYPTGTSLTFGGIAMSPGY
TADASSLAWLGRRLASHGFVVLVINTNSRFDYPDSRASQLSAALNYLRTSSPSAVRARLD
ANRLAVAGHSMGGGGTLRIAEQNPSLKAAVPLTPWHTDKTFNTSVPVLIVGAEADTVAPV
SQHAIPFYQNLPSTTPKVYVELDNASHFAPNSNNAAISVYTISWMKLWVDNDTRYRQFLC
NVNDPALSDFRTNNRHCQ
>PETcan_502
QTSPPTSASLNATAGPLSVSTSSVSSWAARGFGGGTIYYPNATGRYGVVAISPGYTARQS
SIAWLGRRLATHGFVVITIDTNSTLDQPPSRATQLMAALNHVVNNANATVRSRVDASKLA
VAGHSMGGGGSLIAAENNPSLKAAYPLTPWSVSKNYSSVRVPTMIIGADGDSIASVSTHS
RLFYNSLSSNVSKAYGELNNASHFTPNYTNTPIGRYAVTWMKRFVDNDTRYSPFLCGAPH
DSYATRTVFDRYEDNCAY
>PETcan_503
ESPYERGPDPTSASVLDNGTFSLSSTSVSSLVTGFGGGTIYYPTSTTQGTFGGVVLAPGY
TASSSSYSSVARRVASHGFVVFAIDTNSRYDQPDSRGSQILAAVSYLKNSASSTVASRLD
ETRIAVSGHSMGGGGTLAAANQDSSIKAAVALQPWHTDKTWPGIQIPTMIIGAENDSVAP
VASHSIPFYTSMTGAREKAYGEINNGDHFIANTDDDWQGRLFVTWLKRYVDDDTRYSQFL
CPAPSSIYLSDYRNTCPD
>PETcan_504
QAQYQKGPDPTASALERNGPFAIRSTSVSRTSVSGFGGGRLYYPTASGTYGAIAVSPGFT
GTSSTMTFWGERLASHGFVVLVIDTITLYDQPDSRARQLKAALDYLATQNGRSSSPIYRK
VDTSRRAVAGHSMGGGGSLLAARDNPSYKAAIPMAPWNTSSTAFRTVSVPTMIFGCQDDS
IAPVFSHAIPFYNAIPNSTRKNYVEIRNDDHFCVMNGGGHDATLGKLGISWMKRFVDNDT
RYSPFVCGAEYNRVVSSYEVSRSYNNCPY
>PETcan_505
VEIGPAPTSTSLNSDGSFAVSSASVSSSACGSGCAGGTVYYPNTAGSYGVIAVCPGFINT
SSAISWFARRMATHGFVTIAMNTNSRYDFPASRATQLRAVLNYLVNSSSSTIRSRIRSAD
RGVSGYSMGGGGTLLASRDDSTLKTGVPMAPYNSGTISGVNVPQMIIGGSNDSIAPVSSM
ARPFYNNIPSTVKKALAVLNGASHLTFTSYDERAARYGVAFAKRFADGDTRYTPFLCGAE
HTAYATSSRFTEYSSNCPY
>PETcan_601
AANPYQRGPDPTESLLRAARGPFAVSEQSVSRLSVSGFGGGRIYYPTTTSQGTFGAIAIS
PGFTASWSSLAWLGPRLASHGFVVIGIETNTRLDQPDSRGRQLLAALDYLTQRSSVRNRV
DASRLAVAGHSMGGGGTLEAAKSRTSLKAAIPIAPWNLDKTWPEVRTPTLIIGGELDSIA
PVATHSIPFYNSLTNAREKAYLELNNASHFFPQFSNDTMAKFMISWMKRFIDDDTRYDQF
LCPPPRAIGDISDYRDTCPHT
>PETcan_602
AANPYQRGPNPTEASITAARGPFNTAEITVSRLSVSGFGGGKIYYPTTTSEGTFGAIAIS
PGFTAYWSSLEWLGHRLASQGFVVIGIETNTTLDQPDQRGQQLLAALDYLTQRSAVRDRV
DASRLAVAGHSMGGGGSLEAAKARTSLKAAIPLAPWNLDKTWPEVRTPTLIIGGELDAVA
PVATHSIPFYNSLSNAPEKAYLELDNASHFFPNITNTQMAKYMIAWMKRFIDDDTRYTQF
LCPPPSTGLLSDFSDARFTCPM
>PETcan_603
AQNPYERGPAPTEQSVRAERGPFAISQVSVSRLAVSGFGGGTIYYPTSTAEGTFGAVAIA
PGYTASQSSMAWYGPRLASQGFVIFTIDTITTGDQPDSRGRQLLAALDYLTQRSSVRSRV
DASRLGVMGHSMGGGGSLEATVSRPSLQAAIPLTPWNLDKTWPEVRVPTLIIGAENDSIA
PVSSHSEPFYASLPSTLDKAYLELNGASHFAPNVSDTTIARFSISWLKRFIDNDTRYEQF
LCPPPRVSTEISEYRDTCPHSG
>PETcan_604
ASPYERGPAPTSAILEASRGPFATSSINVSSLSVTGFGGGVIYYPTSTAEGTFGAVAISP
GYTASWSSLSWLGPRIASHGFVVIGIETNTRLDQPASRGRQLLAALDYLTERSSVRGRID
SSRLAVAGHSMGGGGSLEAAAARPSLQAAVPLAPWNLDKTWSDVRVPTLIIGGETDSVAP
VATHSIPFYNSIPASSEKAYLELDGASHFFPQTTNTPTAKQMVAWLKRFVDDDTRYEQFL
CPGPSGSAIQEYRNTCPSA
>PETcan_605
AADNPYERGPAPTESSIEALRGPYAVSQTSVSRLAATGFGGGTIYYPTSTADGTFGAVAI
SPGFTALESSISWLGPRLASQGFVVFTIDTLTTVDQPGSRGDQLLAALDYLTQRSSVRGR
IDSSRLGVMGHSMGGGGSLEAAKTRPSLKAAIPMTPWNLDKTWPELRTPTLIFGADADTI
APVATHAKPFYNTLPSSLDRTYIELNNATHFAPNTSNTTIAKYSISWLKRFIDKDTRYEQ
FLCPLPQRSLTIDEAQGNCPHTS
>PETcan_606
SNPYERGPAPTESSVTAVRGYFDTDTDTVSSLVSGFGGGTIYYPTDTSEGTFGGVVIAPG
YTASQSSMAWMGHRIASQGFVVFTIDTITRYDQPDSRGRQIEAALDYLVEDSDVADRVDG
NRLAVMGHSMGGGGTLAAAENRPELRAAIPLTPWHLQKNWSDVEVPTMIIGAENDTVASV
RTHSIPFYESLDEDLERAYLELDGASHFAPNISNTVIAKYSISWLKRFVDEDERYEQFLC
PPPDTGLFSDFSDYRDSCPHTT
>PETcan_607
ADNPYERGPAPTTASIEAARGPYAVSQTTVSSLAVTGFGGGTIYYPTSTGDGTFGAIAVS
PGYTATQSSIAWLGPRLASQGFVVFTIDTLTTLDQPDSRGRQLLAALDHLTQVSSVRTRV
DGSRLGVMGHSMGGGGSLEAAKARPSLQAAIPLTPWNLDKSWPEVGTPTLIVGADGDTVA
PVASHAEPFYSSLPSSLDRAYLELNNATHFTPNSSNTTIAKYGISWLKRFVDNDTRYEQF
LCPLPQPSTTIDEYRGNCPHTS
>PETcan_608
ADNPYARGPEPTTASVEAARGPFAVAQTSVSRYAVSGFGGGTVYYPTTTTAGTFGAVAVS
PGYTARQSSIAWLGPRLASQGFVVITIDTLSTYDQPASRGDQLRAALAYLTQRSSVRARI
DPTRLAVVGHSMGGGGALEAAKDDPSLQAAVPLTGWNLDKTWPEVRTPTLVIGAEDDGVA
PVRSHSEPFYASLPATLDKAYLELRGAGHLAPTVSNTTIATYTLSWLKRFVDDDLRYDRF
LCPAPATSTAIAEYRSTCPY
>PETcan_609
ADNPYQRGPAPTNASIEATRGPYAVSSTSVSSWLVSGFDGGTIYYPTTTADGTFGAVAIS
PGYTAYESSIAWFGERLASQGFVVFTFDTNTTVDQPAQRGDQLLAALDYLTQRSSVRSRV
DASRLGVMGHSMGGGGSLEASKDRPSLKAAIPMTPWNTDKTWSEIRTPTLIFGAENDSVA
PVASHSEPFYSTIPSTTNKMYIELNGASHFAPNSSNTTIAKYSISWLKRFLDNDTRYDQF
LCPLPTSALYIEESRGTCPLR
>PETcan_610
VEATDVHGPDPTEETITAPRGPFDVEQESVSRFEVEGFGGGTIYYPTDTTDGLFSAVSIS
PGYTGTQESMAWYGPRLASHGFVVFTIDTITTTDQPDSRARQLQASLDHLVDDSSVRDRV
DPARLGVMGHSMGGGGSLKAALDNPALQAAIPLTPWHTTKDFSGVRTPTLIIGAQNDTVA
PVSQHAEPFYESLPDDPGKAYLELAGAGHLAPNTPDTTIAKYSLAWLKRFLDDDTRYDQF
LCPPPQDDPEIAEHRSTCPY
>PETcan_611
AEPADVHGPDPTEESITAPRGPFEVDEESVSRLSVSGFGGGTIYYPTDTTDGLFSAVSIS
PGFTGTQETMAWYGPRLASQGFVVFTIDTITTTDQPDSRARQLQASLDYLVNDSDVKDII
DPARLGVMGHSMGGGGSLKAALDNPALKAAIPLTPWHTTKDFSGVQTPTLIIGAQNDTVA
PVSQHAKPFYESLPDDPGKAYLELAGASHLAPNTDNTTIAKFSIAWLKRFLDDDTRYDQF
LCPPPENDDSISDYQSTCPY
>PETcan_612
PGFLGSSSNYAWMGPRLASQGFIVFLINTNTRLDTPPQRGDQLLAALDWLVASSPSAVRT
RLDARRLAVAGHSMGGGGALEASLDRPSLQASLPLQPWHTPASFSGVQVPTMIIGAEADT
TAPVASHAEPFYESLTSASDRAYLELNGADHRVSTTSSTTQAKFMIAWLKRFVDN
>PETcan_701
ANPYERGPNPTDALLEARSGPFSVSEENVSRLSASGFGGGTIYYPRENNTYGAVAISPGY
TGTEASIAWLGKRIASHGFVVITIDTITTLDQPDSRAEQLNAALNHMINRASSTVRSRID
SSRLAVMGHSMGGGGSLRLASQRPDLKAAIPLTPWHLNKNWSSVRVPTLIIGADLDTIAP
VLTHARPFYNSLPTSISKAYLELDGATHFAPNIPNKIIGKYSVAWLKRFVDNDTRYTQFL
CPGPRDGLFGEVEEYRSTCPF
>PETcan_702
AANPYERGPNPTDALLEARSGPFSVSEENVSRLSASGFGGGTIYYPRESNTYGAVAISPG
YTGTEASIAWLGERIASHGFVVITIDTITTLDQPDSRAEQLNAALNHMINRASSTVRSRI
DSSRLAVMGHSMGGGGTLRLASQRPDLKAAIPLTPWHLNKNWSSVTVPTLIIGADLDTIA
PVATHAKPFYNSLPSSISKAYLELDGATHFAPNIPNKIIGKYSVAWLKWFVDNDTRYTQF
LCPGPRDGLFGEVEEYRSTCPF
>PETcan_703
ANPYERGPNPTDALLEARSGPFSVSEENVSRLSASGFGGGTIYYPRENNTYGAVAISPGY
TGTEASIAWLGERIASHGFVVITIDTITTLDQPDSRAEQLNAALNHMINRASSTVRSRID
SSRLAVMGHSMGGGGSLRLASQRPDLKAAIPLTPWHLNKNWSSVRVPTLIIGADLDTIAP
VLTHARPFYNSLPTSISKAYLELDGATHFAPNIPNKIIGKYSVAWLKRFVDNDTRYTQFL
CPGPRDGLFGEVEEYRSTCPF
>PETcan_704
ANPYERGPNPTDALLEARSGPFSVSEENVSRLGASGFGGGTIYYPRENNTYGAVAISPGY
TGTQASVAWLGKRIASHGFVVITIDTITTLDQPDSRARQLNAALDYMINDASSAVRSRID
SSRLAVMGHSMGGGGSLRLASQRPDLKAAIPLTPWHLNKNWSSVRVPTLIIGADLDTIAP
VLTHARPFYNSLPTSISKAYLELDGATHFAPNIPNKIIGKYSVAWLKRFVDNDTRYTQFL
CPGPRDGLFGEVEEYRSTCPF
>PETcan_705
ANPYERGPNPTDALLEARSGPFSVSEERASRFGADGFGGGTIYYPRENNTYGAVAISPGY
TGTQASVAWLGERIASHGFVVITIDTNTTLDQPDSRARQLNAALDYMINDASSAVRSRID
SSRLAVMGHSMGGGGTLRLASQRPDLKAAIPLTPWHLNKNWSSVRVPTLIIGADLDTIAP
VLTHARPFYNSLPTSISKAYLELDGATHFAPNIPNKIIGKYSVAWLKRFVDNDTRYTQFL
CPGPRDGLFGEVEEYRSTCPF
>PETcan_706
ANPYERGPNPTDALLEARSGPFSVSEERASRFGADGFGGGTIYYPRENNTYGAVAISPGY
TGTQASVAWLGKRIASHGFVVITIDTNTTLDQPDSRARQLNAALDYMINDASSAVRSRID
SSRLAVMGHSMGGGGSLRLASQRPDLKAAIPLTPWHLNKNWSSVRVPTLIIGADLDTIAP
VLTHARPFYNSLPTSISKAYLELDGATHFAPNIPNKIIGKYSVAWLKRFVDNDTRYTQFL
CPGPRDGLFGEVEEYRSTCPF
>PETcan_707
ANPYERGPNPTDALLEASSGPFSVSEENVSRLSASGFGGGTIYYPRENNTYGAVAISPGY
TGTEASIAWLGGRIASHGFVVITIDTITTLDQPDSRAEQLNAALNHMINRASSTVRSRID
SSRLAVMGHSMGGGGTPRLASQRPDLKAAIPLTPWHLNKNRSSVTVPTLIIGADLDTIAP
VATHAKPFYNSLPSSISKAYLELDGATHFAPNIPNKIIGKYSVAWLKRFVDNDTRYTQFL
CPGPRDGLFGEVEEYCSTCPF
>PETcan_708
ANPYERGPNPTESMLEARSGPFSVSEERASRLGADGFGGGTIYYPRENNTYGAIAISPGY
TGTQSSIAWLGERIASHGFVVIAIDTNTTLDQPDSRARQLNAALDYMLTDASSSVRNRID
ASRLAVMGHSMGGGGTLRLASQRPDLKAAIPLTPWHLNKSWRDITVPTLIIGADLDTIAP
VSSHSEPFYNSIPSSTDKAYLELNNATHFAPNITNKTIGMYSVAWLKRFVDEDTRYTQFL
CPGPRTGLLSDVDEYRSTCPF
>PETcan_709
ANPYERGPNPTQALLEARSGPFSVSSERAWRLGSDGFGGGTIYYPRENNTYGAVAISPGY
TGTQASVAWLGERIASHGFVVITIDTNTTLDQPDSRARQLDAALDHMLNDASSAVRSRID
RNRLAVMGHSMGGGGTLRLASQRPDLKAAIPLTPWHLNKSWSNVQVPTLIIGADLDTIAP
VLTHAEPFYNSIPTSTRKAYLELDGATHFAPNITNSTIGMYSVAWLKRFVDEDTRYTQFL
CPGPRTGLFSDVEEYRSTCPF
>PETcan_710
ANPYERGPNPTNSSIEALRGPFRVDEERVSRLQARGFGGGTIYYPTDNNTFGAVAISPGY
TGTQSSISWLGERLASHGFVVMTIDTNTTLDQPDSRASQLDAALDYMVEDSSYSVRNRID
SSRLAAMGHSMGGGGTLRLAERRPDLQAAIPLTPWHTDKTWGSVRVPTLIIGAENDTIAS
VRSHSEPFYNSLPGSLDKAYLELDGASHFAPNLSNTTIAKYSISWLKRFVDDDTRYTQFL
CPGPSTGWGSDVEEYRSTCPF
>PETcan_711
ANPYERGPDPTQASLEASRGPFPVSEERVSSPVSGFGGGTIYYPQENNTYGAVAISPGYT
ATQSSVAWLGERIASHGFVVITIDTNTTLDQPDSRADQLEAALDHMVDGASSTVRSRIDR
NRLAVMGHSMGGGGTLRLASRRPDLKAAIPLTPWHLNKSWSNVQVPTLIIGAENDTVAPV
ALHAEPSYTSIPTSTRKAYLELNGASHFAPSVANATIGMYGVAWLKRFVDEDTRYTRFLC
PGPRTGLFSDVEEYRSTCPF
>PETcan_712
ANPYERGPNPTNSSIEALRGPYSVSEDSVSSLVSGFGGGTIYYPTGTNETFGAVAISPGY
TGTQSSISWLGPRLASQGFVVMTIDTNTTLDQPDSRASQLDAALDYMVNRSSSTVRNRID
>PETcan_713
ANPYERGPNPTNSSIEALRGPFRVDEERVSRLQARGFGGGTIYYPTDNNTFGAVAISPGY
TGTQSSISWLGERLASHGFVVMTIDTNTTLDQPDSRASQLDAALDYMVEDSSYSVRNRID
>PETcan_714
ANPYERGPNPTDALLEARSGPFSVSEENVSRLSASGFGGGTIYYPRENNTYGAVAISPGY
TGTEASIAWLGERIASHGFVVITIDTITTLDQPDSRAEQLNAALNHMINRASSTVRSRID
SSRLAVMGHSMGGGGSLRLASQRPDLKAAIPLTPWHLNKNWSSVTVPTLIIGADLDTIAP
VATHAKPFYNSLPSSISKAYLELDGATHFAPNIPNKIIGKYSVAWLKRFVDNDTRYTQFL
CPGPRDGLFGEVEEYRSTCPFY
>PETcan_715
ANPYERGPNPTDALLEASSGPFSVSEENVSRLSASGFGGGTIYYPRENNTYGAVAISPGY
TGTEASIAWLGERIASHGFVVITIDTITTLDQPDSRAEQLNAALNHMINRASSTVRSRID
SSRLAVMGHSMGGGGTLRLASQRPDLKAAIPLTPWHLNKNWSSVTVPTLIIGADLDTIAP
VATHAKPFYNSLPSSISKAYLELDGATHFAPNIPNKIIGKYSVAWLKRFVDNDTRYTQFL
CPGPRDGLFGEVEEYRSTCPF
>PETcan_716
ANPYERGPNPTDALLEARSGPFSVSEENVSRFGADGFGGGTIYYPRENNTYGAVAISPGY
TGTQASVAWLGERIASHGFVVITIDTNTTLDQPDSRARQLNAALDYMINDASSAVRSRID
SSRLAVMGHSMGGGGTLRLASQRPDLKAAIPLTPWHLNKNWSSVRVPTLIIGADLDTIAP
VLTHARPFYNSLPTSISKAYLELDGATHFAPNIPNKIIGKYSVAWLKRFVDNDTRYTQFL
CPGPRDGLFGEVEEYRSTCPFALE
>PETcan_717
ANPYERGPNPTESMLEARSGPFSVSEERASRFGADGFGGGTIYYPRENNTYGAIAISPGY
TGTQSSIAWLGERIASHGFVVIAIDTNTTLDQPDSRARQLNAALDYMLTDASSAVRNRID
ASRLAVMGHSMGGGGTLRLASQRPDLKAAIPLTPWHLNKSWRDITVPTLIIGAEYDTIAS
VTLHSKPFYNSIPSPTDKAYLELDGASHFAPNITNKTIGMYSVAWLKRFVDEDTRYTQFL
CPGPRTGLLSDVEEYRSTCPF

We claim:

1. An engineered organism capable of expressing PET hydrolase enzymes with PET hydrolase activity.

2. The engineered organism of claim 1 wherein the organism is used to degrade PET.

3. The engineered organism of claim 1 wherein the organism is genetically engineered to overexpress PET hydrolase enzymes.

4. A method for identifying PET hydrolase enzymes by identifying nucleic acid sequences from sequenced genomes that are likely to encode for active PET hydrolase enzymes.

5. The method of claim 4 wherein the identified sequences are expressed as engineered PET hydrolase enzymes from a genetically modified organism.

6. The method of claim 4 wherein the engineered organism is genetically engineered to overexpress PET hydrolase enzymes useful for degrading PET.

7. The method of claim 4 further comprising a step of comparing the sequences disclosed herein to sequences of genomes in order to identify PET hydrolases.

8. The method of claim 7 further comprising the step of applying an algorithm to predict the secondary, tertiary and quaternary structure of the PET hydrolases.

9. The method of claim 8 further comprising creating engineered PET hydrolases with increased PET hydrolase activity based upon the predicted tertiary or quaternary structure of the expressed amino acid sequences.

10. A system for identifying PET hydrolase enzymes comprising an engineered organism capable of expressing PET hydrolase enzymes with PET hydrolase activity and comparing the sequences of their corresponding genomes in order to identify PET hydrolases and further comprising the step of applying an algorithm to predict the secondary, tertiary and quaternary structure of the PET hydrolases.

11. The system of claim 10 further comprising creating engineered PET hydrolases with increased PET hydrolase activity based upon the predicted tertiary or quaternary structure of the expressed amino acid sequences.

12. The system of claim 10 wherein the organism is used to degrade PET.
